SEQUENCE LISTING (37 sequences)

SEQ ID NO: 1 - DNA, 11755 bp, synthetic construct
  source 1..11755: mol_type = other DNA
  misc_feature 1..11755: note = 5'LTR-psi-gag-RRE-cPPT-EF1a-Leader-FMC63-CD8hinge-C D8transmembrane-41BB-CD3Zeta-P2A-Puro-WPRE-3'LTR-hU6-BsmBI -Filler-BsmBI-gRNAbackbone-3'LTR
ttaatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagaatcttgagacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccactttggcgccggctcgagggtgcccgggaattctgcaaagatggataaagttttaaacagagaggaatctttgcagctaatggaccttctaggtcttgaaaggagtgggaattggctccggtgcccgtcagtgggcagagcgcacatcgcccacagtccccgagaagttggggggaggggtcggcaattgatccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaggtacccgtacggccaccatggccctccctgtcaccgccctgctgcttccgctggctcttctgctccacgccgctcggcccgaaattgtgatgacccagtcacccgccactcttagcctttcacccggtgagcgcgcaaccctgtcttgcagagcctcccaagacatctcaaaataccttaattggtatcaacagaagcccggacaggctcctcgccttctgatctaccacaccagccggctccattctggaatccctgccaggttcagcggtagcggatctgggaccgactacaccctcactatcagctcactgcagccagaggacttcgctgtctatttctgtcagcaagggaacaccctgccctacacctttggacagggcaccaagctcgagattaaaggtggaggtggcagcggaggaggtgggtccggcggtggaggaagccaggtccaactccaagaaagcggaccgggtcttgtgaagccatcagaaactctttcactgacttgtactgtgagcggagtgtctctccccgattacggggtgtcttggatcagacagccaccggggaagggtctggaatggattggagtgatttggggctctgagactacttactaccaatcatccctcaagtcacgcgtcaccatctcaaaggacaactctaagaatcaggtgtcactgaaactgtcatctgtgaccgcagccgacaccgccgtgtactattgcgctaagcattactattatggcgggagctacgcaatggattactggggacagggtactctggtcaccgtgtccagcaccactaccccagcaccgaggccacccaccccggctcctaccatcgcctcccagcctctgtccctgcgtccggaggcatgtagacccgcagctggtggggccgtgcatacccggggtcttgacttcgcctgcgatatctacatttgggcccctctggctggtacttgcggggtcctgctgctttcactcgtgatcactctttactgtaagcgcggtcggaagaagctgctgtacatctttaagcaacccttcatgaggcctgtgcagactactcaagaggaggacggctgttcatgccggttcccagaggaggaggaaggcggctgcgaactgcgcgtgaaattcagccgcagcgcagatgctccagcctacaagcaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcggggatccggagccacgaacttctctctgttaaagcaagcaggcgacgtggaagaaaaccccggtcctatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgccgtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggagtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgagctagcacgcgttaagtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcgtcgactttaagaccaatgacttacaaggcagctgtagatcttagccactttttaaaagaaaaggggggactggaagggctaattcactcccaacgaagacaagatttcaccattatcgtttcagacccacctcccaaccccgaggggacccagagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggagacggttgtaaatgagcacacaaaatacacatgctaaaatattatattctatgacctttataaaatcaaccaaaatcttctttttaataactttagtatcaataattagaatttttatgttcctttttgcaaacttttaataaaaatgagcaaaataaaaaaacgctagttttagtaactcgcgttgttttcttcacctttaataatagctactccaccacttgttcctaagcggtcagctcctgcttcaatcattttttgagcatcttcaaatgttctaactccaccagctgctttaactaaagcattgtctttaacaactgacttcattagtttaacatcttcaaatgttgcacctgattttgaaaatcctgttgatgttttaacaaattctaatccagcttcaacagctatttcacaagctttcatgatttcttcttttgttaataaacaattttccataatacatttaacaacatgtgatccagctgctttttttacagctttcatgtcttctaaaactaattcataatttttgtcttttaatgcaccaatatttaataccatatcaatttctgttgcaccatctttaattgcttcagaaacttcgaatgcttttgtagctgttgtgcatgcacctagaggaaaacctacaacatttgttattcctacatttgtgccttttaataattctttacaatagcttgttcaatatgaattaacacaaactgttgcaaaatcaaattcaattgcttcatcacataattgtttaatttcagctttcgtagcatcttgttttaataatgtgtgatctatatatttgtttagtttcattttttctcctatatattcatttttaattttaattctttaataatttcgtctactttaactttagcgttttgaacagattcaccaacacctataaaataaatttttagtttaggttcagttccacttgggcgaacagcaaatcatgacttatcttctaaataaaattttagtaagtcttgtcctggcatattatacattccatcgatgtagtcttcaacattaacaactttaagtccagcaatttgagttaagggtgttgctctcaatgatttcattaatggttcaatttttaatttcttttcttctggtttaaaattcaagtttaaagtgaaagtgtaatatgcacccatttctttaaataaatcttctaaatagtctactaatgttttattttgttttttataaaatcaagcagcctctgctattaatatagaagcttgtattccatctttatctctagctgagtcatcaattacatatccataactttcttcataagcaaaaacaaaatttaatccgttatcttcttctttagcaatttctctacccattcatttaaatccagttaaagtttttacaatattaactccatatttttcatgagcgattctatcacccaaatcacttgttacaaaacttgaatatagagccggattttttggaatgctatttaagcgttttagatttgataattttcaatcaattaaaattggtcctgtttgatttccatctaatcttacaaaatgaccatcatgttttattgccattccaaatctgtcagcatctgggtcattcataataataatatctgcatcatgtttaataccatattcaagcggtatttttcatgcaggatcaaattctggatttggatttacaacatttttaaatgtttcatcttcaaatgcatgctcttcaacctcaataacgttatatcctgattcacgtaatatttttggggtaaatttagttcctgttccattaactgcgctaaaaataatttttaaatcttttttagcttcttgctcttttttgtacgtctctgttttagagctagaaatagcaagttaaaataaggctagtccgttatcaacttgaaaaagtggcaccgagtcggtgcttttttaagcttggcgtaactagatcttgagacactgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtacgtatagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagc

SEQ ID NO: 2 - DNA, 13982 bp, Homo sapiens
  source 1..13982: mol_type = genomic DNA; note = RHOG gene
acttccttctcgagcccggagccgctgccgccgcccccagctcccccgcctcggggagggcaccaggtgaggcccggccggggctggcgggtaggggtccgggggaggagagcccggccggcccaggactcggcgcgggcgccggctcatgagtttgctggcggaggccgcgggcgggagcttctcggttcagtccaagtagggcgggcgcggccggccgggcggggcgggaacggggccactgcgagccggcggaggctcggcgagggtggcttcccggcggctgtaccgcctctagggagccgagagggacccctgaggtcggagggtcccgggctccaagataagtgtctctgaagtggaagagctcagagctgggagatcggacgcaggtggggtgtcctcagaagttggtggggggggtgttgagaagtgcgtgaggggagagggcttctgggctggaaaaggcgctccctctgctctggcagttgtgacacttggggtgagaggggcggtgtctggggcctccgaaagctctctccctgtcagatggatgctaatacagacccacactcggtctgcagccccaaggccctctgcactcaggaggacccgggttgggtgggggagccccgccgaccccagtcgttgtctgtatgtctgttttcctgagtcccgaccttacctggagccaagcagtaggtatttagtgagcagacgctgttcctagctgaccagagcctggccttgctcttttgggagtaacttggtcttgccctgtaactggaagtggaaggactctcattcagaggtactgatagtccctgacaagtttttgttgtgtcttttggggacaattgagaaccatctcctagtgttcctcttcattcttagtccctgaattatacacatgctcctgtcgtctccctccacaccttctttagtttctacccacaaatccgccatcactctcttctcatcccttccagtctcttttcctccaaacatacggtcatacctaggccttgacctgtcttcacctaccctgccccatgttcgtcttggtcctctgcaacctgccccatcccttcccttcccagctctggcacccacacacatgcacctcctggtttcttccatcacccctcattccttttgaaccctcagcctgtcaccctctctgtatccttccctcccacccacttatctccagctttggaatcagttgttgaagacttagtgagttctagagtcttttcaaacctccctagcttggagaggcagtctgcagcctcccagtgatagagacggattaaagagctggaaagcctgtgtgtgtgtgtgtgtgtgtgtgtgtttgcgtgtgtgttcgcacgcgcgtgtctgtgtatctacatcttcatctcttagagatggattaaagagatggaaagcctgtgtgtgtgtgtgtgtgtgtgtgtgtgtgtgtgtgtgttcgcgcgcgcgtgtctgtgtgactacatctttatctcttacagacaaaataactgccctcagccttgagagggcccagagcaagggaagccagcagaatgaagggaacctgcagtgagtgaggttgtggttaggcaggaggaagccgtttgtagcagtcagggtagggtgacacaggaataagacttgtaagagaggccgtgggaaatcaaaatcaccccaacccgtggtgcttgaccaagaggccaggcctagaagccggaagacagcctgaaggtccctgactccattacctactagttccagcatcacctggtggaaagaatgggaccagcttgggtgtgatggaaataaagggaggaggcaaatgcaggattctggaggaaagactgcagctggtctaacttcggacttaagaagcggcggaggcaaaagtgtctgctcctttcctgctaccttctggggcctgcctaggctgggtacccgcatgccctgatctgcatcagggccttagtgaccaggaagccctctggagttctactgacccctctgccttcaggcatccttaatactccccatctcacctaggaccctattccttctcaggaattcctcccaaatttaacttcagtccatcctggctatggaggctcttgtttccactctagagctccatcctcaagcttttggggtgcatctgaggatcttgctcctgtctacctcctctagtcatctgtattcatttctctccactagaaaaaaagagaagtagaggggagtaagtgctacagctaaatttaagggctctccctgttccccattctgtcatcttcccatgtcacagtcgtccctctcccatgaattgttcttcttccagtcctgcccttccaaccacttgtctctcttcccatttcttggaggtacagaagtttgggtccgggaagaaatcccagggacctacataagagcccaggttagagaaggatgtgcatacaaggcactttcccagcatcacagcttaataaaaaccccaggagtcccagttcctaggctggcaaaccagtagttactttagtattctgcgtaactgagcaaacccacagggctctgatggtaaagacaagatgggtgagacagggattctcccaggtgagatctggccccattggaagtttgagctaattgcaaaaaaggcatcattggaaaaaatgtgaccagggcgggagagattttaatgggaaaatattggggggtaaagggaggtggtggctacaacctaggaggctgccgctgaagtcttggctaaacaggtatggtcaaggttgtgacatgaagaagggggctcaggcccacactgaagactccttctatatactgtagacccattagtggtgaggtcagtaggggtccgtgacaagagctgaggagggaaaccaaagacaaagcctgggccacctccccagatcagactggttttcctgtccagggtccagcttcctcttttgtcctcatctcatatggggggagggcatctctccaggagctagaggccagaggaggagcttctcagctagggcagggccagagtggctggctattgagtaatgtcagtgctaaatcatgaatgcaccaggatgggctaggcatgagagcctgggaacttggaagcaaaaaggctcagatgtaagtcaacccaccctttggcccattttctgaaggaataaaccatggtagcgttctcctgaatgaggttaaagagatggcccattctcaggccagagttagaatcctcaccttctctcctttgtagaagtagcataagggtggaggctagaagatccggatccttgtcgtagatttgctacctggtcaacttggtttctcttggctctattttagcattaaggagttgcatatcagtacctccaaaggcaacagtgaacatcatggctctgactcttcctcccccaagagactgctcatagcagtgcccctcactctgggcccttgtgtcttaccagcctcaatttgacaggtccgagtagtctgtacatacatatctgtccagggatagtactgccctttccccaagcacaaggactgttagagcttgagagataagaaggcagagaatgttggggattttgccagggggtagtaagatgggggagtaccctgtctcctactccagctctgcctcactgacagtggaaaatagatgccggagtttcctccctggtgttattttcccttgtggaaaccccctcaggcctcgggcggggctaaagggactgggcagccctgggtctctctgtgggtattttcaggtgggggtgccaggatttgcaggatggcacttctgtacagcctcatgtgaaattagcaaaggcaaaggaagtgccacttcacaggcctttggtggggcccagggactgctgagctgctgctaggcggggaggaggaagccagtgtgcacccttcctctgtgctgcgtgggggagggggcagcttccttggctcccccagccagagctgtcctggtcctgaggctcagctttttttttttttttttttttttttttttgagatggagtcttgctctgtcacccaggctggagtgcaatggcgcgatctcagctcaccacaacctccacctcccgggttcaagccattctcctgtgtcagcctcccaggtagcggggactaccggcatgcaccaccaggcccagctaattttttttgtattttttagtagagatggggtttcaccatgctggccaggctggtctcgaattcctgacctcaagtgatctgccagcctcagcctcccaaagtgctgggattacagatgtgagccaccatgcccagctgaggctcaccttttatggaacacttagggtgctgggacagtgagggaagaaggttgggaactaggacttagtgaaagggtcctagactgcccccagccttcccagctaccactgtctcgtttcctagtgggtggcatcaactcttctcttttcctacctgctgctgcaatgtaggatcctctccaaaggctgaaggatgaatgacaagaggtggagagtcctccagcatggattttttctttcttttttttttttttttggagatggagtcttgctctgtggcccaggctggaatgcagtagcatgatctctgctcactgcaacctctgcctcctgggttcaagtgattgtcctccctcagcctcctgagtagctgggattacaggcgtaatcccaccactacgcccagctaatttttgtatttttagtagagacggggtttcaccatgttggccaggctggtttcgaactcctgatctcaagtggtctgcctgccttggcctcccaaagtgctgggattacagacgtgagccacggcacccgggggattttttttttttttttaaggctaggaactgggctctcttgggttcttccctgctgatggggagaaagggaagaaagagactcagcccttggcctggtcccagtcccacactgacctcatgtctcccccagcttcctcctcccagcccagctgcagccagctgcagcccctttgagcctctttatatcacctctcctctattcctcccttcaaaacaaacaaacaaacaaacttgtagagctttggaagtgtttccaacagtgcccctgtcccttgccatgtctttgaccaagggtcctcggatattcccagtctgtagcctgtcgggaccacttttccttcccatcttcctcatcatctctgagctagatctgccagcttcacctctcacccttccagttgaagacaagacagtatagcttatgtgtttattaccccaactttgggctcttcagggtgaatgagaaagaagttggttacttatgtcttgtgtcttctggtcctagaggttcttccttgttcccagcatcagagtctgtggattgatgggggaggaggggtatgtcattctacagcttttgctcagcccttccctgggcttctggaaataaacagaaggtcagactaccctccccctgaagcatccagttagttcagcacaatgaaaagtacggtcaatgctgggaaaggtaaggcagaagagggggttgctgtgatgtcctggagaagctggggaacagtactgggcctgaaggacaggaaggatctgaaggggggtgagtcgcattccaggaagaggaactcccatatgcaaagacatgattgcatcgacttcattcagtcatttactgacagctactacatggtgtgctcagcttagggtgctcagcaaggtccagtcaaccaggaccttgccctctagacactcccattctaataacaagagataagattcacatcagcctctccaagcctgtgagagagacagggaccagactttggataggataagtcacttctgacttggataatttacgctttcctggaagaggcagcaatggtgcatggttccatattactacttggtgtattttacggaagagaatatggaggcccctttgtagaggatcaggattggaatggccagagatggattgtagttataatgaggttcagggagggctgcagaaagggagccccatgttagggaggcaggttatagaaggctgaggtaagttcccatcacctccatactcgcctatcttttaagagtccgttcctcccctcccccactcccagttgggacacttcccctgctgtaacagccacagagatctggcaatcaggctgcttcctttccctcccccattctgggcctgcctgggccacaccccaaggccaggccccactgggtacaagccgggctccagggaccacattcagatagggttagacccccatgggctatacttcctgccggagccaggtcagcctggtactgggaagagagatttttgcctatttcaggcatagcttttgcctgtcattgctgtccgatgagattcagcattgcagtcttgtctgttggggggtgggcagggagtgtccgatactgggcttcagagaagtctctccatatctcagtgatgagggctggtacaagcagatcaaaaggcaatggttgtacagtcctgtgtgcctagccctatacttagaggtttttttttgtcatttagtccagagaagccaaggcttcagtactgaagggcatgtacagctgggaaacttacgtatgactgttggaaaaggagaatcacacagagccaaagcagggttaaaaggggtggccatgggtgcagtggcttactcctgtaatcccagcactttcagaggccaaggcaggtggatcacttgaggccaggtgttcaagaccagcctggccaacatgatgaaatcctgtctctactaaaaatacaaaaattagctgggcatgatggcacatgcatgtaatcccaataactcaggaggctgaggcaggagaatcacttgaacccgggaggtggagattgcagtgagctgagatcgcgacactgcactctaacctgggcgacaggtagccatgcaatggatatccaaagaagaattcagtaaccttatgcaactgtgagacttaagttaggccttaaggagtaaggaagattagagaagggagagggaatggcagcagagggaaaaacaaagagtaaaagttcagaggcaagaatgatcctggcttgtttgcagaggacattgaaaatccctctggaacagggagctgggaactcacacttgcaaagtacctacattgtgcacatattatacattaatgcacttagtccttataacagtgctatgtggtaggtactgtacgttgccactgcatttgaaagataaggaaattgaagcttagagaggcaaatcatcagaagctcagggtcacacagctggtaagaggcagaactaggattcaaacccaagtctacgtaactctaatatcccttatcaaattgtgaagaactctgaatgccagtatgagggaccaggcatgatctgaattgggactaaaaagccagaataagtcattgtaatttgtctaataacatacagatctggccaggcatggtggcccacgcccataatcccagcactttgggagggctagtcaggaggatcacttgagaccaggagttcaagaccagcctgggcaacatggcaagaccctgtctctacaaacaaacaaaaagatgcattaaaaatacaaaaattagccaggtgtggtggtgcctgcctgtagtcccagctactcaggaggctgaggtaggatgccttgagcctgggaggttgagactacagtgagctgtgatggcaccactgcatttcagcctgggcaacagaacaagaccttgctccaaaagaaaaaacaaaacaaaacagattggcaaccattgatttactaaataactttttttttttttgagacatggggtcttgctgtgttgctcaggctggagtgcagtggctactcacagggacgataaccttgactagtgtacacactacaacccttcactcctgggcccagcctccctagtagctgggactgcaggtgcaccactgagctgggcttaatgattcttttgaaaaagccacttagtgaataagtcattgttgaaccccgctccagataggtcacactcctggtttggtgtcctgcagcactgacatcccttcttcctgtttggcacagtttagactctatatgccctcctgaaatgggctatggtaacttattcaacccagttcaggccagaggatttcagaaagaagagttgttttgggagagaggataaaggtctgaactagacctgagtagggaggactgggctgatcccctaccacagatacaggcatactcagtgcttcctgccttctgggatgaaaggctttgctcctgtcacctcctgtgtgtacagcacttcagtttacaaagtgtattcattcccatggtcaggttccttgatgctgcccttgaggctgggaggggtactattattacttctactgtacagataaagaaacagaggctccaggaagggcagtaacttacctgatcaaggtcacacagttagctggtaaggggtaacttgaagactgtatgatgcaccaaggaaaacacaggactttgggtcacaaaactgggtttgtgttctggctcccacactaactagctgtgtgatgttgagttacttttactgttctgaatttgtttcctcatctgtagaataggcatagtactaacaggttgttataaaggtcaaatgacataataacatgtgacctataaaggcttataccatatgagggcttttttatcaagggtcaaggtcggcagataaactcaagacatcagagtcaggtatccagtactcttggccctataccacactgcctttgctctgcttgtttctggacactaccatactcatcatccttttccagtgtcggtcctcgttcccttcccagcccggaagctacgctgtccttcccctctcctttgagagctgatttactgattgctagttgataaccacctctttagagagctgtacttccagtctcttgggccttgtgacagccagccggagttccctccttgccttcctgcctgggccagcccagtgctgggcttttcagtcatactgtcaacacccactctcctgctctgtgtggaactgaagattacaggcccaggagttctgctttgaatgtcaaggcttcccacgtaatccgctgggcacaaataatgccccctgcctccaaggagctttaagtcttagttcatccacctagtgctcttgtaacaaccattttaatctctaattcctggataggaaaactgaggcccagcaagcgtaaatgcctcatgcttggttatagtgagtcagcagagctggggttagaacttaggtctctcaacttccagtctgaggtctctatttgactctccactggcctagagggtccttatgcccttccctgacccactgtctgagggtctctatctgaatgtcctgacgttgcatgcacacgtgcacgcgcacaaacacacacacacactctctctcattcatgcaaataggatggaaatattttttaatgtgaaccagagacacaacttcatagagtgaaaccattcctctcccagctgattccaaggcaaactgtagcctgggagggaggtgggaccaagccctgggtcctttgcagcccccacatgacgaaagtttcttttccacttcccagtggggattttcttccccacttgtcattgatttttgaagcttctgaaccagtggtgagagcccaggctgggggggtaaggggttcccagaccagggtgcagccttgcagtatagcaagtgaaatggagaaaatgggctagtgttgatggtgcttgggtggagccagggataaggaatactcagggatttggttgtgtttaaaacagggctgccccctctcagggagcaggccctggttgtgggagacagtcagggaaaggaagtcacatattgaccaccaagagtcccccatggccaagaggaagtagctgatgagagaaggatccttgccatagcagtgtaaataatcacagggtttctggaagctgtgttccagaactcagaaaagcagttcatctccccgaggccctgtgacctgcagaggtaaccagatgttggctggagctgggactcctcatacttaccccactcataaatacagcagtccatttattgggcacttgctatgtgccatacctcatgctggacatcttctatacattattaactcttactttttatttttttttttttgagacagggtcttgctctgtcgcacaggctggagtgcagtggtgtgatcatagcccactgcagccttgaccaccctgggatcaagtgatgctcccacctcagcctcccgagtagctgggactacaggtgtgtaccaccacaccctggtaattttttcattttttgtagagtcagggttttgccatgttgcccaggctagtctcaaactcctgggctcaagcgatctacctgcctcattctcccaaagtgctaagattacaggcatgagtcaccgtgcctggcctcatttcatattctgagcaattgtaatgcaataggcaagattatccccattttacagatgagaaaattgaggctcagggaacaaatatattgccaagatcacatataactagttggtggagctgtgattagaatacctgcgagtcttactctagcatctatgttcttaatgatggtattttctatccaagagtctggaagtctctaaaatctttcagaggcacatgccaggatctcaggaaatggacagaagattgaggggaaagtcaagtttttgtcttactcttgacttcatccttcctagttgggcctgaaagatttgctccaggccgggcatggtggctcatgcctgtaatcccagcactttttggaaggccaaggcgggcagatcacaaggtcaggagttcaagactagcctgactaacatggtgaaaccccgtctctactaaaaatacaaaaattagctggacgtggtggcatgcgcctgtaatcccagccactcgggagggtgaggcaggagaatcgcttgaacccgggaggtggaggttgcagtgagtcaagattgcgccactacactccagcctgggcgacagagcaagactccgtctcaaaaaagaaaaaaatttgctccaccccaccaagggccttgagagaagatactaatgtctgggagcctcagcctctacctcattcatcataacatacccagaccccagtccaaatcccaggtccaactgaatctccagtattcccctgcattgaattggcaatctgagtgtacatggccagatagggttttcagtacttacaagaatgatggggtggccaggtgtggtggttcacgcctgtaatcccagcactttggaaggctgaggcaggtagatcacctgaggtcaggagcttgagaccagcatggctaacacggcgaaactccatctctactaaaaatataaaaattagccggatgcagtggcacctgcctgtagtcccagccactcgggaggctgaggcaggagacacttgaacccaggaggcggaggttgcagtgagctgagatcacgccactgcactccagcctggacgacagagtgagactccatctcaaaaaaaaaaaaaaaaaaaaatttccccattgccagaataccctggattgggatagggcagctctctaaagaattgagccccttactcagcaggtttcttaactgtgcctttgggtttgtactcagtagccaagataccaggtttggagagggcagggtgggccctgagttgagagaggttctacttatggctgcatcacagagtgacctgggacagctgtcctgtgccaaaacactttccagtctcctggtggaaagggtccatatttagtagaggatctagtagatccctaaatttgtgcactctccttttgtgtctgtatagcctggaaaaaaaaaatagctaatactggccaggcgcagtggctcacgcctgtaatcccagcactttaggaggccaaggcgggcggatcacgaggtcaggagatcgagaccatcctggccaacacggtgaaaccccatctctactaaaaatacaaaaaattagccgggcgtggtggtgggtgcctgtagtcccagctactcgagaggctgaggcagaatggcgtgaacccaggaggcggagcttgcagtaagccgagatgacgccactgcactccagcctgggtgacagagtgagactctgtctcaaaaaaaaaaaaaaaaaaatagctaatacttgtggacgaggcactgttttaaatgctttacatgtattatataatgctcacaatgacccagtgaggtagatattattttatcctcattttactggtaagaaaacttaggcatacagaggttaagcaattttttcagggttacacagctaggaaatagcaaaatcaggatttgaccttagtttgtctggcgccagagtttatgtgctttctaccacattatactgcctctttgcttggcctcagggcctttcattgtgtgtctttctgatgaccgtcttgatgggactttgggatagtgtcaggttaagggaacaggagccggtttccctttgtgtgcccccatcttttcttcccaataggcagaggcctccctaaccaagaatgtcccctttcatctgtccacaggtcactgcagccagaggggtccagaagagagaggaggcactgcctccactacagcaactgcacccacgatgcagagcatcaagtgcgtggtggtgggtgatggggctgtgggcaagacgtgcctgctcatctgctacacaactaacgctttccccaaagagtacatccccaccgtgttcgacaattacagcgcgcagagcgcagttgacgggcgcacagtgaacctgaacctgtgggacactgcgggccaggaggagtatgaccgcctccgtacactctcctaccctcagaccaacgttttcgtcatctgtttctccattgccagtccgccgtcctatgagaacgtgcggcacaagtggcatccagaggtgtgccaccactgccctgatgtgcccatcctgctggtgggcaccaagaaggacctgagagcccagcctgacaccctacggcgcctcaaggagcagggccaggcgcccatcacaccgcagcagggccaggcactggccaagcagatccacgctgtgcgctacctcgaatgctcagccctgcaacaggatggtgtcaaggaagtgttcgccgaggctgtccgggctgtgctcaaccccacgccgatcaagcgtgggcggtcctgcatcctcttgtgaccctggcacttggcttggaggctgcccctgccctccccccaccagttgtgccttggtgccttgtccgcctcagctgtgccttaaggactaattctggcacccctttccagggggttccctgaatgcctttttctctgagtgcctttttctccttaaggaggcctgcagagaaaggggctttgggctctgcccccctctgcttgggaacactgggtattctcatgagctcatccaagccaaggttggacccctccccaagaggccaacccagtgccccctcccattttccgctactgaccagttcatccagctttccacacagttgttgctgcctattgtggtgccgcctcaggttaggggctctcagccatctctaacctctgccctcgctgctcttggaattgcgcccccaagatgctctctcccttctccaatgagggagccacagaatcctgagaaggtgaatgtgccctaacctgctcctctgtgcctaggccttacgcatttgctgactgactcagcccccatgcttctggggacctttcctacccccatcagcatcaataaaacctcctgtctccagtg

SEQ ID NO: 3 - DNA, 576 bp, Homo sapiens
  source 1..576: mol_type = other DNA; note = RHOG CDS
atgcagagcatcaagtgcgtggtggtgggtgatggggctgtgggcaagacgtgcctgctcatctgctacacaactaacgctttccccaaagagtacatccccaccgtgttcgacaattacagcgcgcagagcgcagttgacgggcgcacagtgaacctgaacctgtgggacactgcgggccaggaggagtatgaccgcctccgtacactctcctaccctcagaccaacgttttcgtcatctgtttctccattgccagtccgccgtcctatgagaacgtgcggcacaagtggcatccagaggtgtgccaccactgccctgatgtgcccatcctgctggtgggcaccaagaaggacctgagagcccagcctgacaccctacggcgcctcaaggagcagggccaggcgcccatcacaccgcagcagggccaggcactggccaagcagatccacgctgtgcgctacctcgaatgctcagccctgcaacaggatggtgtcaaggaagtgttcgccgaggctgtccgggctgtgctcaaccccacgccgatcaagcgtgggcggtcctgcatcctcttgtga

SEQ ID NO: 4 - protein, 191 aa, Homo sapiens
  source 1..191: note = RHOG
MQSIKCVVVGDGAVGKTCLLICYTTNAFPKEYIPTVFDNYSAQSAVDGRTVNLNLWDTAGQEEYDRLRTLSYPQTNVFVICFSIASPPSYENVRHKWHPEVCHHCPDVPILLVGTKKDLRAQPDTLRRLKEQGQAPITPQQGQALAKQIHAVRYLECSALQQDGVKEVFAEAVRAVLNPTPIKRGRSCILL

SEQ ID NO: 5 - RNA, 20 nt, synthetic construct
  source 1..20: mol_type = other RNA
gcaggcacgtcttgcccaca

SEQ ID NO: 6 - RNA, 20 nt, synthetic construct
  source 1..20: mol_type = other RNA
gccaagcagatccacgctgt

SEQ ID NO: 7 - RNA, 20 nt, synthetic construct
  source 1..20: mol_type = other RNA
gcaggcacgtcttgcccaca

SEQ ID NO: 8 - RNA, 20 nt, synthetic construct
  source 1..20: mol_type = other RNA
gaatgctcagccctgcaaca

SEQ ID NO: 9 - RNA, 20 nt, synthetic construct
  source 1..20: mol_type = other RNA
cgcacagtgaacctgaacct

SEQ ID NO: 10 - RNA, 20 nt, synthetic construct
  source 1..20: mol_type = other RNA
ccaccaccacgcacttgatg

SEQ ID NO: 11 - RNA, 20 nt, synthetic construct
  source 1..20: mol_type = other RNA
tcccacaggttcaggttcac

SEQ ID NO: 12 - RNA, 20 nt, synthetic construct
  source 1..20: mol_type = other RNA
gccgaggctgtccgggctgt

SEQ ID NO: 13 - RNA, 20 nt, synthetic construct
  source 1..20: mol_type = other RNA
gtgcgctacctcgaatgctc

SEQ ID NO: 14 - RNA, 20 nt, synthetic construct
  source 1..20: mol_type = other RNA
cgcccacgcttgatcggcgt

SEQ ID NO: 15 - RNA, 20 nt, synthetic construct
  source 1..20: mol_type = other RNA
gccaagcagatccacgctgt

SEQ ID NO: 16 - RNA, 20 nt, synthetic construct
  source 1..20: mol_type = other RNA
gctctcaggtccttcttggt

SEQ ID NO: 17 - RNA, 20 nt, synthetic construct
  source 1..20: mol_type = other RNA
gacaccctacggcgcctcaa

SEQ ID NO: 18 - RNA, 20 nt, synthetic construct
  source 1..20: mol_type = other RNA
gatgtgcccatcctgctggt

SEQ ID NO: 19 - RNA, 20 nt, synthetic construct
  source 1..20: mol_type = other RNA
gaaacagatgacgaaaacgt

SEQ ID NO: 20 - RNA, 20 nt, synthetic construct
  source 1..20: mol_type = other RNA
cgccgtcctatgagaacgtg

SEQ ID NO: 21 - RNA, 20 nt, synthetic construct
  source 1..20: mol_type = other RNA
gctgtaattgtcgaacacgg

SEQ ID NO: 22 - RNA, 20 nt, synthetic construct
  source 1..20: mol_type = other RNA
cctgaacctgtgggacactg

SEQ ID NO: 23 - RNA, 20 nt, synthetic construct
  source 1..20: mol_type = other RNA
gagggtaggagagtgtacgg

SEQ ID NO: 24 - RNA, 20 nt, synthetic construct
  source 1..20: mol_type = other RNA
gaaaacgttggtctgagggt

SEQ ID NO: 25 - RNA, 20 nt, synthetic construct
  source 1..20: mol_type = other RNA
tagttgtgtagcagatgagc

SEQ ID NO: 26 - RNA, 20 nt, synthetic construct
  source 1..20: mol_type = other RNA
agcgcgcagagcgcagttga

SEQ ID NO: 27 - RNA, 20 nt, synthetic construct
  source 1..20: mol_type = other RNA
atatgtcccagatagcactg

SEQ ID NO: 28 - RNA, 20 nt, synthetic construct
  source 1..20: mol_type = other RNA
aagtggttgataaacccacg

SEQ ID NO: 29 - RNA, 20 nt, synthetic construct
  source 1..20: mol_type = other RNA
tgtcacactccagttcactg

SEQ ID NO: 30 - RNA, 20 nt, synthetic construct
  source 1..20: mol_type = other RNA
agatacgcctggttgcccag

SEQ ID NO: 31 - RNA, 20 nt, synthetic construct
  source 1..20: mol_type = other RNA
gaagccagtagagctcaaag

SEQ ID NO: 32 - RNA, 20 nt, synthetic construct
  source 1..20: mol_type = other RNA
gcggatcgagactggcaacg

SEQ ID NO: 33 - RNA, 20 nt, synthetic construct
  source 1..20: mol_type = other RNA
tgctcagtggaaaccacgaa

SEQ ID NO: 34 - RNA, 20 nt, synthetic construct
  source 1..20: mol_type = other RNA
gaacctgagctgctctgacg

SEQ ID NO: 35 - RNA, 19 nt, synthetic construct
  source 1..19: mol_type = other RNA
cacgtgtagtcgatctgat

SEQ ID NO: 36 - DNA, 61 bp, synthetic construct
  source 1..61: mol_type = other DNA
ttggaccctcgtacagaagctaatacgactcactataaggaaataagagagaaaagaagag

SEQ ID NO: 37 - DNA, 71 bp, synthetic construct
  source 1..71: mol_type = other DNA
tttttttttttttttttttttttttttttttttttttttttttttttttttttttctagaccctcacttcc

(19) United States
(12) Patent Application Publication
(10) Pub. No.: US 2026/0284106 A1
(43) Pub. Date: Sep. 24, 2026

(54) METHOD FOR OPTIMIZING T CELLS FOR IMMUNO-THERAPY

(71) Applicant: CEMM - FORSCHUNGSZENTRUM FÜR MOLEKULARE MEDIZIN GMBH, Wien (AT)

(72) Inventors: Paul DATLINGER, Wien (AT); Evgeniia PANKEVICH, Wien (AT); Christoph BOCK, Wien (AT)

(73) Assignee: CEMM - FORSCHUNGSZENTRUM FÜR MOLEKULARE MEDIZIN GMBH, Wien (AT)

(21) Appl. No.: 19/136,462

(22) PCT Filed: Dec. 8, 2023
(86) PCT No.: PCT/EP2023/084998

§ 371 Date: Jun. 6, 2025

(30) Foreign Application Priority Data
Dec. 9, 2022 (EP) 22212656.7

(51) Int. Cl. A61K 35/17; A61K 31/7105; A61K 40/11; A61K 40/31; A61K 40/42; A61K 48/00; A61P 35/02; C07K 14/705; C07K 16/00; C12N 5/074; C12N 5/0783

(52) U.S. Cl. CPC A61K 35/17; A61K 31/7105; A61K 40/11; A61K 40/31; A61K 40/42; A61K 48/00; A61P 35/02; C07K 14/70517; C07K 16/00; C12N 5/0607; C12N 5/0636; C07K 2317/53; C07K 2317/622; C07K 2319/03; C12N 2510/00

(57) ABSTRACT

The present invention is in the field of immunotherapy, in particular cell-based immunotherapies, e.g. for the treatment of cancer. Specifically, the present invention concerns the depletion or inactivation of RHOG and/or the suppression of RHOG expression and/or activity in T cells (in particular human T cells), e.g., CAR T cells, in vitro and/or in vivo. Therefore, the present invention provides RHOG modulators which are suitable for suppressing, impairing or abolishing the expression and/or activity of RHOG in a mammalian N cell such as a T cell. Moreover, the present invention relates to medical uses of such a RHOG modulator or T cells (e.g. CAR T cells) which comprise such a RHOG modulator or which have been contacted with such a RHOG modulator, e.g. for the treatment of cancer, as well as uses of such a RHOG modulator for culturing and/or producing T cells inviro, e.g., for adoptive cell transfer. Combinations of a RHOG modulator according to the invention and synthetic antigen receptors such as CARs are also encompassed by the present invention. Furthermore, the present invention provides specific CRISPR gRNAs and specific combinations of CRISPR gRNAs and CRISPR modifiers.

The present invention is in the field of immunotherapy, in particular cell-based immunotherapies, e.g. for the treatment of cancer. Specifically, the present invention concerns the depletion or inactivation of RHOG and/or the suppression of RHOG expression and/or activity in T cells (in particular human T cells), e.g., CAR T cells, in vitro and/or in vivo. Therefore, the present invention provides RHOG modulators which are suitable for suppressing, impairing or abolishing the expression and/or activity of RHOG in a mammalian cell such as a T cell. Moreover, the present invention relates to medical uses of such a RHOG modulator or T cells (e.g. CAR T cells) which comprise such a RHOG modulator or which have been contacted with such a RHOG modulator, e.g. for the treatment of cancer, as well as uses of such a RHOG modulator for culturing and/or producing T cells in vitro, e.g., for adoptive cell transfer. Combinations of a RHOG modulator according to the invention and synthetic antigen receptors such as CARs are also encompassed by the present invention. Furthermore, the present invention provides specific CRISPR gRNAs and specific combinations of CRISPR gRNAs and CRISPR modifiers.

Accordingly, the present invention relates, inter alia, to a pharmaceutical composition comprising a T cell, said T cell comprising a RHOG modulator according to the invention, and/or an inactivating mutation in at least one allele of an endogenous RHOG gene. In addition, the present invention relates to a CAR T cell population in which expression of RHOG is suppressed. Furthermore, the invention relates to an engineered mammalian cell comprising (I) a RHOG modulator according to the invention and/or an inactivating mutation in at least one allele of an endogenous RHOG gene, and (II) a synthetic antigen receptor, preferably a chimeric antigen receptor (CAR). The invention also relates to a pharmaceutical composition comprising a RHOG modulator according to the invention, as well as a RHOG modulator according to the invention for use in an immunotherapy, wherein said RHOG modulator is to be administered to a subject, preferably a human. In addition, the present invention relates to a cell culture comprising a RHOG modulator according to the invention, as well as production methods comprising a step of contacting mammalian cells with a RHOG modulator according to the invention. Further aspects of the present invention are described herein.

Cancer is a group of diseases involving abnormal cell proliferation with the potential to invade or spread to other parts of the body. Cancer is a major public health problem worldwide and is the second leading cause of death in the United States. Although extensive research relating to cancer has been conducted and pharmaceuticals have been found there is still a significant interest in the development of new cancer treatment strategies.

Cell-based therapies such as CAR T cell therapies have a huge potential to extend treatment options for cancer⁶ and a wide range of other diseases⁹. CAR T cells may be derived from the patient's own T cells that are expanded ex vivo and transduced with viral vectors encoding the chimeric antigen receptor. Instead of viral vectors, transposons such as piggyBac or Sleeping Beauty are also used in the field, or the CAR is integrated into an endogenous gene locus such as TRAC. CAR T cells are a genetically engineered cell product, re-infused into patients for therapy, e.g., to fight their cancer. Currently, there is great interest to develop next-generation CAR T cell therapies that harbor additional genetic modifications, such as CRISPR-mediated knockouts of PD1¹² and components of the TCR¹³ (e.g. TRAC, TRBC). Other beneficial genetic alterations were found by serendipity. In one patient with chronic lymphocytic leukemia, the complete remission was driven by a single CAR T cell clone in which the epigenetic regulator TET2 had been disrupted by the lentiviral integration¹⁴.

The groundbreaking success of CAR T cell treatment in B cell precursor acute lymphoblastic leukemia (pre-B ALL) and large B cell lymphoma has revolutionized the field of immunotherapy⁸. However, durable remission is only achieved in a small subset of patients, and low treatment efficacy has been reported for solid tumors. These limitations^{10,11} are due to insufficient target cell recognition, low antigen density, poor CAR T cell proliferation and persistence, compromised T cell phenotypes and effector function, T cell exhaustion, or T cell fratricide.

Thus, there is still a need to provide means and methods to improve cell based therapies. The technical problem underlying the present invention is the provision of means and methods to improve cell based therapies.

The technical problem is solved by provision of the embodiments characterized in the claims and as provided herein below.

Accordingly, the present invention relates, inter alia, to a pharmaceutical composition comprising a T cell, said T cell comprising (a) a RHOG modulator that is suitable for suppressing, impairing or abolishing the expression and/or activity of RHOG in a T cell, and/or (b) an inactivating mutation in at least one allele of an endogenous RHOG gene.

In particular, the expression and/or activity of RHOG is suppressed or abolished in a T cell according to the invention.

The present invention is, inter alia, based on the surprising finding that RHOG depletion or inactivation enhances T cell function, as illustrated in the appended Examples. In particular, the inventors surprisingly found that knock-out of RHOG in CAR T cells increased the fitness of the cells regardless of whether they were stimulated via a chimeric antigen receptor (CAR) or via a T cell receptor (TCR); see e.g., Examples 1 and 2. More specifically, it has been found in context of the present invention that RHOG depletion or inactivation results in stronger T cell proliferation in response to TCR and CAR stimulation, a much more proliferative and metabolically active T cell phenotype, a favorable cell signaling and beneficial features of memory T cells.

Strikingly, and unexpectedly, the inventors further found that knock-out of RHOG in CAR T cells strongly enhanced the anti-tumor/anti-cancer properties of the CAR T cells in xenograft mouse models; see Example 2 and FIGS. 16 and 17. This surprising and advantageous therapeutic effect of RHOG depletion or inactivation was observed in both, a leukemia xenograft model and a solid tumor model, demonstrating the great efficacy and versatility of RHOG depletion or inactivation in T cells such as CAR T cells. The inventors envision that suppressing, impairing or abolishing the expression and/or activity of RHOG in a T cell will be broadly useful to enhance T cell therapies, e.g., for the treatment of cancer, autoimmune disorders or chronic infections.

RHOG is a GTPase with a wide variety of functions reported in T cells—from involvement in immune synapse formation and cell migration to regulation of T cell signaling and anergy. Several studies have examined the effect of RHOG depletion on T cells, e.g., in knockout mice (Vigorito (2004), Molecular and Cellular Biology 24 (2): 719-29), patients with deleterious mutations, knockout cell lines and knockdown in primary CD8 T cells (Kalinichenko (2021, Blood 137 (15): 2033-45). However, the results from the various scientific papers are inconclusive. Vigorito (2004) generated a homozygous knockout mouse for RHOG, describing a mere mild phenotype, and suggesting that RHOG is functionally redundant. Mokhtar (2022) also mentions the redundancy of RhoG in T cells, and further indicates that lack of RHOG may marginally increase T cell proliferation; see, Mokhtar (2022), Frontiers in Immunology 13 (February): 845064. The person skilled in the art immediately recognizes that a “marginal” increase of T cell proliferation does not provide any meaningful insights on T cell functionality, let alone on any potential therapeutic effects of the T cells. Furthermore, the person skilled in the art would not consider a functionally redundant gene as a suitable target for improving immunotherapies. Some prior art studies even suggest that RHOG may be a positive regulator of T cell function (Mokhtar (2022)). Further studies suggest that RHOG inactivation results in immunodeficiency (Kalinichenko (2021)), from which a person skilled in the art concludes that RHOG inactivation is detrimental for the normal function of therapeutic T cells. Because RHOG plays a critical role in multiple T cell functions, the impact of changes in RHOG activity on T cell therapy could not be predicted. Moreover, the function of RHOG specifically in CAR T cells has not been studied at all.

The screening data described herein further show that the RHOG knockout has a particularly strong positive effect on CAR T proliferation (MAGeCK MLE normalized beta value of 0.9990, see FIG. 13) and also a positive effect on T cell proliferation via a TCR (MAGeCK MLE normalized beta value of 0.4057, see FIG. 13 and FIG. 18B). This is a particularly surprising finding that no one could have possibly expected. Even more surprisingly, the RHOG knockout enhanced the therapeutic effect of T cell therapy, in particular CAR T cell therapy, and prolonged survival in in vivo cancer models, more effectively as compared to other gene knockouts, e.g., the positive control knockout PRDM1 (see e.g., FIGS. 16-17).

Accordingly, the present invention, inter alia, provides improved therapeutic T cells such as CAR T cells by suppressing, impairing or abolishing the expression and/or activity of RHOG, i.e. RHOG depletion and/or inactivation, e.g., via “knockdown” or “knockout” of RHOG.

Herein and in context of the present invention, the term “RHOG”, may refer to a RHOG gene and/or a RHOG protein, especially in the context of the term “expression”. For example, suppression of RHOG gene expression usually also suppresses RHOG protein expression. The term “activity” of RHOG, as used herein, refers primarily to the RHOG protein activity.

Preferably herein, the RHOG gene (in particular, the human wildtype (WT) RHOG gene) refers to the genomic DNA sequence according to the NCBI Reference Sequence “NC_000011.10”, and, in particular to the genomic DNA sequence as set forth in SEQ ID NO: 2. Furthermore, the coding sequence (CDS) of the RHOG gene (in particular, the CDS of the human WT RHOG gene), as used herein, preferably refers to the sequence shown in SEQ ID NO: 3. Accordingly, the RHOG protein (in particular, the human WT RHOG protein), as used herein, has, preferably, the amino acid sequence shown in SEQ ID NO: 4.

A modifier for CRISPR knockout, e.g. a CRISPR modifier comprising an endonuclease, (for example SpCas9; Cas12a, aka Cpf1; SaCas9) may be used in combination with gRNAs designed to target early (e.g. in the first quarter of the coding sequence) in the RHOG coding sequence, considering the PAM requirements of the CRISPR modifier. In one particularly preferred embodiment, at least one gRNA is selected from the 8 CRISPR knockout gRNAs targeting RHOG that were tested extensively in context of the present invention and as illustrated in the appended Examples (see Table 8 and SEQ ID NO: 19-26), and the modifier is, preferably, SpCas9. During cell manufacturing, the CRISPR modifier and gRNA(s) can be delivered as RNP, or as synthetic mRNA+gRNA.

Furthermore, the present invention provides improved therapeutic T cells by epigenome editing, such as CRISPRi and CRISPRoff at a RHOG gene locus.

Specifically, as an alternative method for RHOG depletion or inactivation that does not involve the formation of double-strand breaks in therapeutic cells, epigenome editing, for example CRISPR Inhibition (CRISPRi) or CRISPRoff may be used. This strategy involves the recruitment of a nuclease-deficient CRISPR protein (for example dCas9) fused to a repressive protein domains to at least one relevant promoter or enhancer element of a RHOG gene. Key promoter and enhancer elements required for the expression of RHOG in the context of CAR T cells may be identified, e.g., by profiling RHOG knockout and SafeHarbor control CAR T cells in response to repeated antigen stimulation by ATAC-seq and/or RNA-seq (see, e.g., FIG. 18). Repressive domains that may be used in context of the present invention may be derived from transcription factors or epigenetic modulators. Furthermore, one or more repressive domains from one or more transcription factors or epigenetic modulators may be combined. Examples of repressive domains that are useful for targeting promoters include, inter alia, KRAB, Zim3, MeCP2, DNMT3A, and DNMT3L. Alternatively, RHOG enhancers may be targeted, for example by fusing a nuclease-deficient CRISPR protein to one or more repressive protein domains derived from epigenetic modifiers such as histone deacetylases (HDACs), epigenetic writers of H3K27me3 or H3K9me3, for example, HDAC1, HDAC2, HDAC3, HDAC6, HDAC8, SIRT1, HDAC4, HDAC5, HDAC9, HDAC10, SIRT2, EZH2, G9A/EHMT2, EHMT1, NSD2/WHSC1/MMSET, EZH1, SUV39H1, SIN3A, mSin3a, and/or HDAC11. Any of the above-mentioned repressive domains for targeting promoters or enhancers may be used in context of the present invention, in particular, in an endonuclease deficient protein fused to a repressive domain, as described herein below.

In preferred embodiments, the endonuclease deficient protein (e.g. dCas9) is fused to a KRAB domain and a further repressive domain as described herein, preferably a Zim3 domain and/or a MeCP2 domain.

A CRISPRi or CRISPRoff approach as described herein may be particularly advantageous for clinical applications. Such an approach may include: (1) obtaining an ATAC-seq dataset in RHOG Knockout versus control CAR T cells to derive key promoter and enhancer elements for the RHOG gene (2) designing gRNAs targeting these promoter and/or enhancer elements (3a) electroporating mRNA for an epigenetic CRISPR editor and epigenetic editing gRNA or alternatively (3b) electroporating an RNP complex consisting of epigenetic CRISPR editing protein plus gRNA targeting a key epigenetic feature of a RHOG gene, and (4) expanding therapeutic T cells with standard methods and administering them to patients.

In addition, the present invention, inter alia, provides improved therapeutic T cells by CRISPR base editing or prime editing.

Specifically, a further particularly attractive method for RHOG depletion or inactivation that does not involve the induction of double-strand breaks and is potentially safer for use in the clinic, is changing a RHOG sequence by CRISPR base editing or prime editing. As illustrated in the appended Examples, the inventors found that base editing with a variety of editors supplied as electroporated messenger RNAs (ABEmax, an NGG-ABE; AncBE4max, an NGG-CBE; ABEmax7.10-SpRY, an SpRY-ABE; CBE4max-SpRY, an SpRY-CBE) results in surprisingly high editing efficiencies in primary human T cells and CART cells within each gRNA's editing window. In the base editing screens performed in context of the invention, the inventors used all possible gRNAs targeting RHOG, based on the PAM-requirements of each base editor. The base editing screening results are ideal for developing a clinical base editing strategy for RHOG. It is thought that such a strategy prioritizes base editing gRNAs with high z-score/proliferation score in the screens. A list of the top 15 highest scoring gRNAs from the base editing screens is provided (see Table 6). Their use may be highly beneficial to produce clinical-grade therapeutic T cells. Furthermore, selecting a gRNA for clinical base editing may involve prioritizing gRNAs with a defined editing window that minimizes bystander mutations. Importantly, the subset of base editing gRNAs that introduces Stop codons into the RHOG sequence are of particular interest for clinical base editing due to their known and complete effect; see also Table 6. A single base editing gRNA, or a mixture of a few such gRNAs targeting strategic sites in the RHOG sequence may be used. Methods to produce base edited therapeutic T cells can be implemented by a person skilled in the art based on published protocols, e.g., as described in Chiesa, N Engl J Med. 2023 Sep. 7; 389(10):899-910, and as described herein; see e.g. Example 3. Preferably, a base editor and gRNA may be delivered to the cells as electroporated synthetic RNAs, including modifications in each RNA molecule that optimize stability and/or editing, and/or minimize the immune response in therapeutic cells. Another preferred example is the electroporation of an RNP complex comprising a base editor protein in complex with a synthetic base editing gRNA.

As used herein, the RHOG modulator according to the invention is suitable for suppressing, impairing or abolishing the expression and/or activity of RHOG in a mammalian cell such as a T cell. Accordingly, the RHOG modulator of the according to the invention may be used to deplete or inactive RHOG in a mammalian cell such as a T cell, as described herein.

RHOG depletion or inactivation can be quantified in multiple ways. In the in vivo experiments shown in Example 2, the RHOG genomic locus contained out-of-frame mutations in at least 30% of T cells. This level of editing led to significant enhancement of CAR T cell treatment in comparison to standard unmodified CAR T cells. Therefore, even lower levels of editing may be beneficial for therapy. For example, complete RHOG depletion or inactivation (e.g. by genomic mutation in the RHOG gene) even in a small fraction of T cells may be used for a clinical product. Alternatively, any level of functional alteration (e.g., by a RHOG inhibitor) in a fraction of T cells or CAR T cells may improve the (CAR) T cell therapy performance. The inventors have established that the in vitro proliferation of T cells in response to TCR or CAR stimulation can predict the therapy effect in vivo. As illustrated in the appended Examples, a 10% increase of in vitro expansion (FIG. 18B) was sufficient to translate into a better therapeutic response in an in vivo leukemia model (FIG. 16). Therefore, in vitro expansion can reflect the effect of RHOG inactivation achieved by any means, as described herein, including RHOG gene editing, RHOG inhibition, or preselection of a specific cell population.

More specifically, as used herein and in context of the present invention, e.g. in context of the pharmaceutical composition described above, a RHOG modulator may comprise
(i) a gene editing molecule and, optionally, a gene editing modifier,
(ii) an RNA interference (RNAi) molecule, and/or
(iii) a DNA molecule for targeted integration into an endogenous RHOG gene.

In particular, said gene editing molecule and/or said gene editing modifier target a RHOG gene in a mammalian cell such as a T cell. More specifically, said gene editing molecule and/or said gene editing modifier is/are suitable for introducing an inactivating mutation in an endogenous RHOG gene in a T cell according to the invention, as described herein.

The term “gene editing molecule” is used herein in the broadest sense and refers to all molecules that may edit, engineer or modulate the genetic content (e.g. a RHOG gene) of a cell or organism. Preferably, the gene editing molecule is a CRISPR guide RNA (gRNA).

The term “gene editing modifiers” is used herein in the broadest sense and refers to all molecules that induce genetic editing, engineering or modulation. Preferably, the gene editing modifiers are CRISPR modifiers.

In preferred embodiments, the gene editing molecule comprises a CRISPR guide RNA (gRNA) and said gene editing modifier comprises a CRISPR modifier as described herein. The combination of a gRNA and a CRISPR modifier is also referred to as a “CRISPR system” herein. In some preferred embodiments, the CRISPR guide RNA targeting a RHOG gene or CDS has a sequence selected from the group consisting of: SEQ ID NO: 5 to 26, e.g., SEQ ID NO: 25.

In some embodiments, the CRISPR modifier comprises an endonuclease. Preferably, the endonuclease is SpCas9, Cas12a, SaCas9, SpG or SpRY.

In combination with a CRISPR endonuclease as described herein, or a nucleic acid encoding said CRISPR endonuclease, the CRISPR guide RNA targeting a RHOG gene or CDS may, preferably, have a sequence selected from the group consisting of: SEQ ID NO: 19 to 26, preferably SEQ ID NO: 25 (see, e.g., Table 8).

A CRISPR modifier according to the invention may comprise
(I) an endonuclease, preferably wherein the endonuclease is SpCas9, Cas12a, SaCas9, SpG or SpRY;
(II) a prime editor, preferably wherein said prime editor comprises a Cas9 nickase fused to a reverse transcriptase,
(III) a base editor such as a cytosine base editor (CBE), an adenine base editor (ABE), an cytosine-to-guanine base editor (CGBEs) and/or a simultaneous adenine and cytosine base editor (ACBEs), preferably wherein said base editor comprises a Cas9 nickase fused to a nucleoside deaminase, more preferably an ABEmax, an NGG-ABE, an AncBE4max, an NGG-CBE, an ABEmax7.10-SpRY, a SpRY-ABE, a CBE4max-SpRY, and/or a SpRY-CBE; and/or
(IV) an endonuclease deficient protein fused to a repressive domain, preferably wherein the nuclease deficient protein is dCas9, and, preferably, wherein said repressive domain is derived from a transcription factor or epigenetic modulator.

A prime editor according to the invention and as described herein is preferably combined with a prime editing guide RNA (pegRNA). CRISPR prime editors are well known in the art and the skilled person can readily provide suitable pegRNAs. CRISPR base editors are also well known in the art. Suitable CRISPR base editors are further described herein, for example, in the appended Examples (see, e.g. Table 7) and any of these may be used in context of the present invention.

In combination with a CRISPR base editor as described herein, or a nucleic acid encoding said CRISPR base editor, the CRISPR guide RNA targeting a RHOG gene or CDS may, preferably, have a sequence selected from the group consisting of: SEQ ID NO: 5 to 18. Particularly effective combinations of base editors and guide RNA targeting RHOG are described herein, e.g., in Tables 6 and 7.

Furthermore, the RHOG modulator according to the invention, in particular the gene editing molecule and/or said gene editing modifier, may comprise a transcription activator-like effector nuclease (TALEN) and/or a zinc finger nuclease. TALENs and zinc finger nucleases are suitable alternatives to a CRISPR system, especially a CRISPR system comprising an endonuclease, as described herein. A TALEN or a zinc finger nuclease combines the functions of a gene editing molecule and a gene editing modifier as described herein in one molecule. Thus, a TALEN or a zinc finger nuclease may be considered as a gene editing molecule and/or as a gene editing modifier, as used herein.

In context of the present invention, a DNA molecule for targeted integration into an endogenous RHOG gene, preferably, comprises a sequence that is homologous or identical to a sequence within a RHOG gene, e.g., in a T cell according to the invention. In particular, said DNA molecule for targeted integration into an endogenous RHOG gene is suitable for homology directed repair (HDR) at the RHOG gene locus.

Furthermore, the molecule for targeted integration into an endogenous RHOG gene according to the invention may comprise an open reading frame (ORF). Preferably, said ORF may encode a synthetic antigen receptor, more preferably, a chimeric antigen receptor (CAR), as described herein. Accordingly, said ORF may be knocked-in to the RHOG gene locus thereby destroying the RHOG ORF and effectively causing a knock-out of RHOG gene.

A gene editing molecule and/or a gene editing modifier, e.g., a CRISPR modifier comprising an endonuclease, a TALEN and/or a zinc finger nuclease as described herein, may be advantageously used in combination with a molecule for targeted integration into an endogenous RHOG gene according to the invention, especially, to increase the efficiency of homology directed repair and integration of an ORF (a CAR ORF) into a RHOG gene.

CRISPR modifiers may be also used for RHOG knockdown. Additionally, knockdown can be established via expression of short hairpin RNA (shRNA) or small interfering RNA (siRNA), which can be achieved, for example, by stable integration of DNA expression constructs into the genome or by RNA delivery via transfection (e.g., electroporation or lipofection).

Accordingly, in some embodiments, the RHOG modulator of the invention comprises an RNA interference (RNAi) molecule. In particular, the RNAi molecule according to the invention is suitable for suppressing the translation of a RHOG mRNA and/or for degrading a RHOG mRNA in a mammalian cell such as T cell according to the invention. Preferably, the RNAi molecule comprises a small-interfering RNA (siRNA), and/or a small-hairpin RNA (shRNA).

All nucleotide and amino acid sequences used in the invention can be optimized to reduce immunogenicity in therapy, e.g., by various de-immunization methods that are well established in the art.

An inactivating mutation according to the invention may suppress, impair or abolish the expression of a RHOG gene or RHOG protein in a mammalian cell such as T cell according to the invention, in particular, as compared to a corresponding mammalian cell (e.g. a T cell) comprising exactly two alleles of a RHOG gene each having a sequence as set forth in SEQ ID NO: 2. Furthermore, an inactivating mutation may suppress, impair or abolish the activity of a RHOG protein in a mammalian cell such as T cell according to the invention, in particular, as compared to a RHOG protein consisting of a sequence as set forth in SEQ ID NO: 4.

Furthermore, an inactivating mutation according to the invention may comprise at least one deletion, insertion and/or point mutation in the RHOG gene, preferably relative to the reference sequence of the RHOG gene as shown in SEQ ID NO: 2. Said at least one deletion, insertion and/or point mutation may be located in the coding sequence (CDS), an untranslated region (UTR) and/or a regulatory sequence (e.g. a promoter or enhancer) of the RHOG gene. Preferably, at least one deletion, insertion and/or point mutation is located in the coding sequence (CDS) of the RHOG gene, more preferably relative to the reference RHOG CDS of as shown in SEQ ID NO: 3.

Furthermore, said deletion(s), insertion(s) or point mutation(s) may cause(s) a frameshift and/or a premature stop codon in the RHOG ORF or CDS, and/or at least 5%, 10%, 20%, 50%, 70%, 90% or 100% of the RHOG CDS as set forth in SEQ ID NO:3 may be deleted. A RHOG ORF or CDS comprising a frameshift or a premature stop codon may be translated into a RHOG protein with a reduced functionality or a protein with no functionality.

In some embodiments, the point mutation(s) in the RHOG gene is/are selected from the group consisting of: V8I, V9M, C18R or C18Y, T501, W56Ter, P109F, R120K, T125I, K147G, Q148R or Q148Ter, R153C, S158L, R184H, G185K, and A172V in the sequence set forth in SEQ ID NO:4. The term “Ter”, as used herein, refers to the replacement of a codon by a stop codon and the corresponding absence of the amino acid encoded by the original codon.

Another suitable point mutation which may inactive RHOG is E171K in the sequence set forth in SEQ ID NO:4, and which may be also used in context of the present invention.

An inactivation mutation as described herein may be introduced into one or more or all RHOG alleles of a mammalian cell (e.g. a T cell) according to the invention.

In preferred embodiments, a T cell according to the invention comprises a synthetic antigen receptor. Preferably herein and in context of the present invention, a synthetic antigen receptor is a chimeric antigen receptor (CAR), as described herein.

A synthetic antigen receptor, as described herein, may be a single polypeptide or may be comprised of multiple polypeptides. Multi-chain antigen receptors (i.e. synthetic antigen receptors comprised of multiple polypeptides) are described, e.g., in WO 2023/118610, which is incorporated herein by reference in its entirety. Preferably, the synthetic antigen receptor, in particular a CAR according to the invention, is a single polypeptide.

In principle, any synthetic antigen receptor (e.g. a CAR) described in the prior art or herein to function in a T cell may be used in context of the present invention, regardless of the exact design and the antigen specificity. The terms “chimeric antigen receptor” or “CAR” are used herein as commonly used in the art and any “CAR” described or yet to be developed is encompassed by these terms.

In particular, a synthetic antigen receptor according to the invention (e.g. a CAR) comprises an antigen-binding domain, a transmembrane domain, and an intracellular signalling domain. The antigen-binding domain is, in particular, at the external side of the cell of the invention.

Furthermore, a synthetic antigen receptor according to the invention may comprise a leader peptide, and/or a hinge domain. In particular the hinge domain is located between the antigen-binding domain and the transmembrane domain.

Preferably, synthetic antigen receptor according to the invention, and in particular the antigen-binding domain comprised therein, specifically binds to CD19, BCMA, GD2, HER2, CD123, CD138, CD20, CD22, CD38 CDK5, IgK, LeY, NKG2D, ROR1, WT1, c-met, CAIX, CD70, CEA, EGFR, EpCAM, EphA2, FAP, GD2 HER2, IL13RA2, LeY, MAGEA3, MAGEA4, MART1, Mesothelin, MUC1, MUC16, NY-ESO-1, PD-L1, PSCA, PSMA, VEGFR2, CD171, EGFRVvIII, FR, Glypican-3, CD23, CD32B, CD70, CD72, CD37, CD79b, CD133, FcμR, Siglec-6, TSLPR, BAFF-R, GPRC5D, LMP1, SLAMF7, or GPRC5D, more preferably to CD19; see also, e.g., Pierelli, Cells. 2022 June; 11(11): 1804.

In some embodiments, the synthetic antigen receptor according to the invention, and in particular the antigen-binding domain comprised therein, specifically binds to a neoantigen, e.g., a tumor neoantigen.

In principle, any antigen-binding domain (e.g. from a single-chain variable fragment (scFv), a full-length antibody, a Fab fragment, a F(ab)2 fragment, a peptide mimetic, a DARPin, or a monobody may be used in the synthetic antigen receptor according to the invention. In preferred embodiments, the antigen-binding domain is a single-chain variable fragment (scFv).

In some embodiments, the transmembrane domain and the hinge domain of the synthetic antigen receptor are from CD8.

In particular herein, the intracellular signalling domain of the synthetic antigen receptor comprises an activating domain. Preferably, the intracellular signalling further comprises at least one co-stimulatory domain.

In preferred embodiments, the intracellular signalling domain according to the invention, in particular the activating domain, comprises a CD3ζ chain, or at least one, preferably all, immunoreceptor tyrosine-based activation motifs (ITAMs) from CD3ζ.

In further embodiments, the intracellular signalling domain according to the invention, in particular the activating domain, comprises an FceRIg chain, or at least one, preferably all, ITAMs from FceRIg.

In some embodiments, the intracellular signalling domain comprises at least one co-stimulatory domain selected from the co-stimulatory domains of CD28, CD27, OX40, 4-1BB, ICOS, CD40, CD40L, Toll-like receptors (TLRs), MyD88, and IL-15Rα; see, e.g., Weinkove, Clin Transl Immunology. 2019; 8(5): e1049. In some preferred embodiments, said intracellular signalling domain comprises the co-stimulatory domains of CD28 and 4-1BB, or the co-stimulatory domains of CD28 and OX40, preferably the co-stimulatory domains of CD28 and 4-1BB.

In a particularly preferred embodiment, the synthetic antigen receptor according to the invention, in particular the CAR, is a polypeptide comprising an scFv (e.g. specifically binding CD19), a hinge domain from CD8, a transmembrane domain from CD8, and an intracellular signalling domain comprising co-stimulatory domains of CD28 and 4-1BB and a CD3ζ chain, and optionally a leader peptide.

In particular, herein and in context of the present invention, the synthetic antigen receptor is not a conventional T cell receptor (TCR). However, a T cell according to the invention may comprise only a conventional TCR, or a TCR in addition to a synthetic antigen receptor according to the invention.

Thus, a T cell according to the invention may comprise a TCR, and/or a synthetic antigen receptor as described herein.

The T cell according to the invention may be a CD8+ T cell or a CD4+ T cell. A CD8+ T cell also refers to a cytotoxic lymphocyte (CTL). A population of T cells as described herein may comprise CD8+ and/or CD4+ T cells of the invention.

It has been surprisingly found in context of the present invention, that RHOG depletion or inactivation enhances the expression of the memory phenotype markers CD62L and CD45RO in T cells, as illustrated in the appended Examples. A memory phenotype is an advantageous property of T cells such as CAR T cells because it is known that memory-like T cells have a particularly high efficacy in therapy.

Thus, preferably herein, the T cell according to the invention has a memory phenotype, preferably a central memory phenotype. Accordingly, the T cell according to the invention may express the memory phenotype markers CD62L and CD45RO.

Furthermore, the T cell of the invention may be activated. Accordingly, the T cell of the invention may express the activation marker CD69.

Furthermore, the T cell of the invention may express CD107a, IL-2, and/or INFγ.

Preferably, the T cell of the invention is not exhausted or anergic. Accordingly, the T cell of the invention does, preferably, not express PD1, LAG3 and/or TIM3 or may show a low or reduced expressed of PD1, LAG3 and/or TIM3.

Many types of cells may be used a starting material for use in the present invention. This includes but is not limited to (1) autologous T cells obtained from a patient by apheresis (2) off-the-shelf T cells of primary origin, for example derived from healthy donors (3) off-the-shelf T cells of cell line origin (4) iPSC cultures differentiated into relevant immune cell types, such as T cell subsets, and (5) artificially produced, synthetic cells with cytotoxic phenotypes. As described herein, therapeutic T cells with RHOG inactivation may be also generated in vivo, for example by delivery of viral vectors or lipid particles carrying genome editing molecules into a patient's bloodstream, or relevant immune sites.

Accordingly, the T cell of the invention may be (a) a primary T cell such as an autologous T cell or an allogeneic T cell, (b) from a T cell line, or (c) derived from pluripotent stem cells, preferably induced pluripotent stem cells (iPSCs). It is routine for the person skilled in the art to obtain any of these T cells.

Most preferably, herein and in context of the present invention, the cell, i.e. the mammalian cell, (e.g. a T cell or a CAR T cell) is a human cell, (e.g. a human T cell or a human CAR T cell). However, it also possible that the cell is from another mammal such as a horse, a cow, a sheep, a pig, a dog, a cat, a monkey, an ape, a polar bear, a tiger, a rhinoceros, a mouse, a rat, a guinea pig, or an alpaca etc. Preferably herein, a subject is to be treated with cells of the same species. It is particularly preferred in context of the present invention that human cells (e.g. human T cells or human CAR T cells according to the invention) are used for treating a human subject, in particular, a human patient.

Therefore, the invention further relates to a T cell, said T cell comprising (a) a RHOG modulator that is suitable for suppressing, impairing or abolishing the expression and/or activity of RHOG in a T cell, as described herein, and/or (b) an inactivating mutation in at least one allele of an endogenous RHOG gene, as described herein. In particular, the expression and/or activity of RHOG is suppressed or abolished in the T cell according to the invention. Preferably said T cell comprises a synthetic antigen receptor, preferably a CAR, as described herein.

The invention also relates to a population of T cells comprising a plurality of the T cells of the invention. Said population of T cells may comprise a plurality of T cells of the same type and/or a plurality of different types of T cells according to the invention, as described herein.

It is also contemplated in context of the present invention that other immune cells wherein RHOG has a biological function, in particular natural killer cells (NK cells) or macrophages may be used instead of T cells.

The pharmaceutical composition according to the invention (in particular as described herein above) may comprise a T cell population, wherein said T cell population comprises a plurality of the T cells of the invention. A T cell population of the invention may comprise a plurality of the same type of T cells of the invention and/or a plurality of different types of T cells of the invention. For example, the T cell population of the invention may comprise CD8+ T cells and/or CD4+ T cells. As another example, the T cell population of the invention may comprise T cells without a synthetic antigen receptor and/or T cells comprising a synthetic antigen receptor as described herein.

Furthermore, at least 1%, 5%, 10%, 20%, 30%, 40%, 50%, 60%, 70%, 80%, 90%, 95%, 99% or 100% of the cells in the T cell population of the invention may comprise (a) a RHOG modulator that is suitable for suppressing, impairing or abolishing the expression and/or activity of RHOG in a T cell, and/or (b) an inactivating mutation in at least one allele of an endogenous RHOG gene, as described herein.

In preferred embodiments, the T cell population according to the invention is a CAR T cell population comprising a plurality of T cell(s) comprising a CAR as described herein. Preferably, the expression of RHOG is suppressed in said T cell population.

Furthermore, at least 1%, 5%, 10%, 20%, 30%, 40%, 50%, 60%, 70%, 80%, 90%, 95%, 99% or 100% of the cells in the T cell population of the invention may comprise a synthetic antigen receptor, preferably a CAR, as described herein.

A pharmaceutical composition of the invention may further comprise a pharmaceutically acceptable excipient, carrier and/or diluent, as described herein.

Pharmaceutically acceptable excipients, carriers and diluents (e.g. buffer solutions such as a isotonic saline solution) for T cells are well known in the art and any of these may be used in context of the present invention. A merely exemplary buffer solution is the CliniMACS® Formulation (Miltenyi Biotec), wherein 100 mL thereof contain: Sodium chloride (473 mg), Sodium gluconate (452 mg), Sodium acetate trihydrate (331 mg), Trisodium citrate dihydrate (289 mg), Potassium chloride (34 mg), Magnesium chloride hexahydrate (28 mg) and Human serum albumin (2500 mg).

Furthermore, suitable excipients, carriers and diluents are described, e.g., in WO 2020/161224, which is incorporated by reference as far as it pertains to pharmaceutically acceptable excipients, carriers and diluents.

Furthermore, the T cells (e.g. CAR T cells) according to the invention may be cryopreserved.

Accordingly, the pharmaceutical composition of the invention may comprise a cryopreservation agent, in particular a pharmaceutically acceptable cryopreservation agent. Pharmaceutically acceptable cryopreservation agents for T cells are well known in the art and any of these may be used in context of the present invention.

In addition to RHOG, further gene knockouts have been identified in context of the present invention which may also enhance T cell functions, as described herein below and as illustrated in Example 1.

Accordingly, the invention further relates to a CAR T cell population in which expression of RHOG, HAVCR2, VAMP4, SLC35B3, TRAF1, NOTCH2, PHF8, DUSP6, DAW1, ZNF16, TCF4, PRSS3, LACTB, TMTC3, CD300LB, MORC3 and/or CDKN2A is suppressed. Preferably, the expression of RHOG is suppressed in the CAR T cell population of the invention.

Furthermore, the present invention relates to a CAR T cell, wherein a RHOG gene is suppressed or deleted.

The invention also relates to a pharmaceutical composition comprising a CAR T cell or a CAR T cell population of the invention, and optionally further a pharmaceutically acceptable excipient, carrier and/or diluent, as described herein.

Based on the data provided herein in context of the present invention, it is expected that RHOG will be useful in a wide range of clinical indications, in particular, where increased T cell proliferation, increased T cell metabolic activity and/or T cell memory phenotypes are desired; see, e.g., Example 2.

Accordingly, a T cell according to the invention, a T cell population according to the invention or a pharmaceutical composition according to the invention may be for use in preventing, ameliorating or treating a disease or disorder, as described herein, preferably in a human patient.

Furthermore, the invention relates to corresponding methods of treatment, wherein an effective amount of a T cell according to the invention, a T cell population according to the invention or a pharmaceutical composition according to the invention is administered to a subject in need thereof.

Preferably herein, and in context of the present invention, a subject or patient to be treated is a human.

As used herein, “treatment” (and grammatical variations thereof such as “treat” or “treating”) refers to clinical intervention in an attempt to alter the natural course of the individual being treated. Desirable effects of treatment include, but are not limited to, prophylaxis, preventing occurrence or recurrence of disease or symptoms associated with disease, alleviation of symptoms, diminishment of any direct or indirect pathological consequences of the disease, decreasing the rate of disease progression, amelioration or palliation of the disease state, improved prognosis and cure.

In particular, the T cell, T cell population or pharmaceutical composition of the invention may be for use in treating or ameliorating a cancer, a tumour or a cancerous disease, an autoimmune disorder, a chronic bacterial or viral infection, or Graft Versus Host Disease (GVHD), as described herein.

Preferably herein, the disease or disorder is a cancer, tumour or cancerous disease, more preferably a cancer.

The cancer, tumour or cancerous disease may be a blood cancer, a liquid tumor or a hematological tumor such as a leukemia, a lymphoma or a myeloma. For example, the blood cancer, the liquid tumor or the hematological tumor may be selected from the group consisting of: an acute Lymphocytic Leukemia (ALL), a B-Cell Leukemia such as B-Cell Acute Lymphocytic Leukemia, B-Cell Chronic Lymphocytic Leukemia, Nodal Marginal Zone B-Cell Lymphoma, Extranodal Marginal Zone B-Cell Lymphoma (Mucosa-Associated Lymphoid Tissue or MALT-Lymphoma), or Splenic Marginal Zone B-Cell Lymphoma, a T-Cell Leukemia such as T-Cell Acute Lymphocytic Leukemia, Chronic Lymphocytic Leukemia (CLL) (e.g. Refractory CLL or Relapsed CLL), an Acute Myelocytic Leukemia (AML) (e.g. Refractory AML or Relapsed AML), a Chronic Myelocytic Leukemia (CML), or Hairy Cell Leukemia, a B-Cell Lymphoma such as Diffuse Large B-Cell Lymphoma, Marginal Zone B-cell Lymphoma, Hodgkin Lymphoma (in particular, B-Cell Hodgkin Lymphoma), Non-Hodgkin Lymphoma (in particular, B-Cell Non-Hodgkin Lymphoma), Primary Mediastinal B-Cell Lymphoma, a T-cell Lymphoma such as a Peripheral T-Cell Lymphoma (PTCL), an Angioimmunoblastic T-Cell Lymphoma (AITL), a Follicular Lymphoma, a Mantle Cell Lymphoma, Burkitt Lymphoma, Anaplastic Large Cell Lymphoma (ALCL), a Lymphoblastic Lymphoma such as Acute Lymphoblastic Lymphoma, Natural Killer Cell Lymphomas, Waldenstrom Macroglobulinemia, Multiple Myeloma (e.g. Refractory Multiple Myeloma or Relapsed Multiple Myeloma), a Myelodysplastic Syndrome, a Plasma Cell Neoplasm, Sezary Syndrome, Mycosis Fungoides, and POEMS Syndrome (Crow-Fukase Syndrome).

Furthermore, the cancer, tumour or cancerous disease may be a solid tumor. For example the solid tumor may be selected from the group consisting of: Pancreatic Cancer, Metastatic Pancreatic Cancer, Gastric Cancer, Ovarian Cancer such as Epithelial Ovarian Cancer, Lung Cancer such as Small-Cell Lung Cancer or Non-Small Cell Lung Cancer, Colorectal Cancer such as Metastatic Colorectal Cancer, Glioblastoma Multiforme (GBM) such as Recurrent Glioblastoma Multiforme (GBM), Hepatocellular Carcinoma, Breast Cancer such as Metastatic Breast Cancer, Human Epidermal Growth Factor Receptor 2 Positive Breast Cancer (HER2+ Breast Cancer) or Triple-Negative Breast Cancer (TNBC), Neuroblastoma, Prostate Cancer such as Castration-Resistant Prostate Cancer (CRPC) or Metastatic Castration-Resistant Prostate Cancer (mCRPC), Esophageal Cancer, Liver Cancer, Melanoma such as Metastatic Melanoma, Bile Duct Cancer (Cholangiocarcinoma), Sarcoma such as Osteosarcoma, Glioma, Malignant Mesothelioma such as Malignant Pleural Mesothelioma, Endometrial Cancer, Kidney Cancer such as Renal Cell Carcinoma (e.g. Metastatic Renal Cell Carcinoma), Fallopian Tube Cancer, Adenocarcinoma Of The Gastroesophageal Junction, Pancreatic Ductal Adenocarcinoma, Cervical Cancer, Bladder Cancer, Nasopharyngeal Cancer, Peritoneal Cancer, and Head And Neck Cancer.

The autoimmune disorder may be, for example, inter alia, Systemic Lupus Erythematosus, Systemic Sclerosis or Lupus Nephritis.

The chronic bacterial or viral infection may be, for example, Human Immunodeficiency Virus (HIV) Infection.

In some preferred embodiments, a CAR T cell of a CAR T cell population of the invention or a pharmaceutical composition of the invention comprising such a CAR T cell or CAR T cells is for use in the prevention, treatment or amelioration of cancer, as described herein.

Furthermore, RHOG may be depleted or inactivated in T cells in a subject, e.g. a human patient, i.e., in vivo, as described herein. To this end, a RHOG modulator that is suitable for suppressing, impairing or abolishing the expression and/or activity of RHOG in a T cell in a subject, as described herein may be administered to a subject, for example, systemically (e.g. to the blood stream) or locally (e.g. a relevant immune site where T cells are present). RHOG depletion or inactivation in vivo may enhance any immune therapies where T cells are involved. Therefore, administration of a RHOG modulator according to the invention to a subject may be suitable for treating any of the diseases or disorders in a subject as described herein.

Any of the RHOG modulators described herein may be, in principle, suitable for in vivo administration and used for this purpose.

Furthermore, the RHOG modulator according to the invention may be co-administered with T cells (prior to, simultaneously with or subsequently to the administration of the T cells), e.g., with a T cell or T cell population according to the present invention.

Accordingly, the present invention further relates to a pharmaceutical composition comprising a RHOG modulator that is suitable for suppressing, impairing or abolishing the expression and/or activity of RHOG in a T cell in a subject, in particular, as described herein. Optionally, the pharmaceutical composition further comprises a T cell or a T cell population, in particular, as described herein. Preferably, the T cell is a CAR T cell, and, preferably, the T cell population is a CAR T cell population, as described herein.

The pharmaceutical composition may further comprise a pharmaceutically acceptable excipient, carrier and/or diluent, as described herein.

In context of the present invention, e.g., in context of direct in vivo administration of a RHOG modulator, the RHOG modulator may comprise
(i) a CRISPR guide RNA (gRNA) or a DNA molecule encoding said gRNA as described herein, and/or a CRISPR modifier or a nucleic acid molecule (e.g. an RNA such as an mRNA or a DNA) encoding said CRISPR modifier;
(ii) a ribonucleoprotein (RNP) comprising a CRISPR modifier and a CRISPR guide RNA, as described herein; (iii) a TALEN or a zinc finger nuclease a described herein, or a nucleic acid molecule encoding said TALEN or said zinc finger nuclease;
(iv) an siRNA and/or an shRNA, as described herein; and/or
(v) a RHOG inhibitor, as described herein.

In some embodiments, the RHOG modulator comprises at least one viral vector encoding the DNA molecule encoding a gRNA as described herein and/or the nucleic acid molecule encoding a CRISPR modifier as described herein.

In some embodiments, the RHOG modulator comprises a viral vector encoding a TALEN or a zinc finger nuclease as described herein.

Any viral vector that is suitable for delivery of a nucleic acid, in particular for gene delivery in vivo, may be, in principle, used in context of the present invention. Various suitable viral vectors are known in the art and/or described herein.

Preferably, the viral vector may be a lentiviral vector, a retroviral vector, an adeno-associated virus (AAV) vector, or an Adenoviral vector.

In some preferred embodiments, the nucleic acid molecule encoding a CRISPR modifier, a TALEN or a zinc finger nuclease according to the invention is an RNA, more preferably an mRNA. Such an RNA may be, for example, electroporated or transfected into cells in vivo, e.g., by means of lipid nanoparticles (LNP) or lipoplex (LPX), as described herein and as is well established in the art. Accordingly, the RNA is, preferably, comprised in a lipid particle, more preferably a lipid nanoparticle (LNP) or a lipoplex (LPX).

In some embodiments, the RHOG modulator comprises a transposon or transposon system, comprising a CRISPR guide RNA and/or a CRISPR modulator, as described herein.

Furthermore, the RHOG modulator according to the invention, e.g., in context of direct in vivo administration of a RHOG modulator, may comprise a RHOG inhibitor, as described herein. In the context of RHOG inhibitors, the term “RHOG” refers, in particular, to a RHOG protein, as described herein.

While in the experiments described in the appended Examples, RHOG was permanently inactivated in cells via genetic knockout, temporary RHOG inactivation may be also relevant for clinical use. For example, knockdown and/or RHOG inhibition may be done temporarily, either during T and CAR T cell production (e.g., for increased yield and improved phenotype of the injected product), as described herein, e.g. in context of the inventive production methods, cell culture and cell culture methods, and/or during the treatment, as described herein, e.g., in context of directed in vivo administration of a RHOG modulator.

Hence, pharmacological and/or transient inhibition of RHOG as described herein, in particular in context of a RHOG inhibitor, may be used for improved production of therapeutic T cells, and/or as combination treatment for T cell therapies, as described herein.

RHOG inhibitors are known in the art and, in principle, any of these may be used in context of the present invention. The RHOG inhibitor according to the invention may comprise a small molecule or an antigen-binding molecule such as an antibody. The antigen-binding molecule may be, inter alia, a full-length antibody, a Fab fragment, a F(ab)2 fragment, or an scFv), a peptide such as a peptide mimetic, a DARPin, a monobody or an aptamer.

Specifically, based on the surprising findings of the inventors, inhibition of RHOG with small molecules or biologics could be employed (1) during the cell manufacturing stage, e.g., to yield a higher number of therapeutic T cells with desirable phenotypes, as described herein, e.g. in context of the inventive production methods, and cell cultures, (2) as a combination treatment administered in addition to therapeutic T cells, e.g., as described herein context of in vivo administration on a RHOG modulator, or (3) a combination of both.

Small molecules include but are not limited to direct inhibitors of RHOG's active (GTP-binding) domain and allosteric inhibitors binding elsewhere on the protein, as described herein. Small molecules or biologics targeting upstream Guanine nucleotide Exchange Factors (GEFs) that catalyze the exchange of GDP for GTP and are activators of RHOG activity may be also used. GEFs activating RHOG in the context of TCR and CAR activation may be identified from the proliferation screens described herein, and by smaller, focused screens using the same experimental strategy. Biologics targeting RHOG can include, but are not limited to antibodies, scFvs, peptides. For all drug modalities, the base editing data provided herein will be useful to inform the drug development process, drug design, protein design, design of small molecules or biologics by generative artificial intelligence. For all drug modalities, the base editing data may be useful for predicting treatment response, and to select patient groups for treatment.

Accordingly, the RHOG inhibitors according to the invention may be direct or indirect inhibitors of RHOG. For example, compounds targeting upstream activators of RhoG, such as guanine nucleotide exchange factors (GEFs), maybe be suitable in context of the present invention for inhibiting RhoG activation.

Furthermore, Rac1 inhibitors are also proposed to be inhibitors of RhoG due to structural homology.

Thus, in some preferred embodiments, the RHOG inhibitor is also a Rac1 inhibitor, more preferably, NSC23766 or NSC23766 trihydrochloride (i.e. N6-[2-(4-Diethylamino-1-methyl-butylamino)-6-methyl-pyrimidin-4-yl]-2-methyl-quinoline-4,6-diamine trihydrochloride).

Furthermore, the RHOG inhibitor according to the invention may block the binding of RHOG to GTP, and/or the RHOG inhibitor according to the invention may inhibit the RHOG active GTPase site in a competitive or non-competitive manner.

In addition, the RHOG inhibitor according to the invention may bind to the GTP-binding domain of RHOG, or the RHOG inhibitor according to the invention may be an allosteric inhibitor of RHOG.

Exemplary RHOG inhibitors include, inter alia: NSC23766 trihydrochloride, Rac1 Inhibitor (CAS 1177865-17-6), plinabulin (e.g. from Beyond Spring Inc), i.e. (3Z,6Z)-3-benzylidene-6-[(5-tert-butyl-1H-imidazol-4-yl)methylidene]piperazine-2,5-dione, compounds 1,5 and 6 as disclosed in Arnst, Oncotarget. 2017 May 23; 8(21): 34586-34600, Rac1 inhibitors described in WO 2014/198909, RhoG inhibitors (prenyl protein transferase inhibitors, famesyl protein transferase inhibitors and geranylgeranyl protein transferase inhibitors, toxins A and B from C. difficile, C. sordellii lethal toxin, botulinum toxin C 3, Staphylococcal toxin EDFN, the small molecule NSC23766, siRNAs, dominant negative mutants, ribozymes, antibodies and blocking peptides) as recited in claim 5 of WO 2008/067288, antisense RhoG inhibitors as described in U.S. Pat. No. 5,965,370. Further RHOG inhibitors are described in WO 2021/257828 and WO 2005/051392. All the above cited documents are incorporated herein by reference in their entirety.

Plinabulin is an inhibitor of particular interest which may be used in context of the present invention.

As already mentioned above, the RHOG modulator according to the invention may be used for immunotherapies, in particular immunotherapies in which T cells are involved, e.g. to treat cancer or any of the other diseases or disorders mentioned herein in context of the present invention. In one aspect of the invention, the RHOG modulator is to be directly administered to a subject, preferably a human patient.

Accordingly, the present invention further relates to a RHOG modulator according to the invention for use in an immunotherapy, wherein said RHOG modulator is suitable for suppressing, impairing or abolishing the expression and/or activity of RHOG in a T cell, and wherein said RHOG modulator is to be administered to a subject, and, in particular, wherein T cells are to be contacted with said RHOG modulator in said subject, as described herein.

In some embodiments, the RHOG modulator is to be administered to the subject in combination with a T cell or a T cell population, preferably a CAR T cell or a CAR T cell population, in particular, as described herein. The RHOG modulator may be administered prior to, simultaneously with or subsequently to administration of the T cell or T cell population.

In particular, the immunotherapy according to the invention may be for use in preventing, ameliorating or treating a disease or disorder, as described herein, e.g, cancer.

As already mentioned above, a RHOG modulator (e.g. RHOG inhibitor) according to the invention is also particularly useful for optimizing T cells in vitro, i.e., in a cell culture or cell culture method. In particular, T cells with an improved phenotype as described herein (e.g. T cells showing a memory phenotype, greater proliferation and/or metabolically active phenotype) may be produced in vitro and then adoptively transferred into a subject. Moreover, a RHOG modulator according to the invention (e.g. a RHOG inhibitor) may be employed to increase the yield in T cell therapy production. This has high clinical relevance, because higher cell proliferation results in higher cell yields and, therefore, more types of patients can be treated. For some patient groups, and especially heavily pre-treated cancer patients, it is often difficult or impossible to produce enough T cells of sufficient quality, such as CAR T cells, and RHOG inactivation can increase the likelihood of production success.

Accordingly, the present invention further relates to a cell culture comprising
(i) a medium supplemented with a RHOG modulator that is suitable for suppressing, impairing or abolishing the expression and/or activity of RHOG in a mammalian cell, in particular a T cell, as described herein and
(ii) a population of mammalian cells comprising cells comprising a synthetic antigen receptor, preferably a chimeric antigen receptor (CAR), as described herein.

The present invention also relates to a method of culturing mammalian cells, said method comprising a step of culturing a population of mammalian cells, as described herein in a medium supplemented with a RHOG modulator that is suitable for suppressing, impairing or abolishing the expression and/or activity of RHOG in a mammalian cell, in particular a T cell, as described herein; and wherein said population of mammalian cells comprises cells comprising a synthetic antigen receptor, preferably a chimeric antigen receptor (CAR).

As described herein, the population of mammalian cells according to the invention may comprise T cells, T cell precursor cells, or pluripotent stem cells, preferably wherein said pluripotent stems cells are iPSCs.

Preferably, the mammalian cells according to the invention comprise T cells. The medium for culturing T cells according to the invention may further comprise IL-2, IL-7 and/or IL-15, and/or any other cytokines generally used for T cell expansion. Furthermore, the medium for culturing T cells according to the invention may comprise a T cell stimulation reagent such as beads comprising anti-CD3 and anti-CD28 antibodies, beads comprising anti-CD2, anti-CD3 and anti-CD28 antibodies, a soluble antigen, beads comprising an antigen, and/or lipid vesicles and/or cells presenting an antigen, wherein said antigen is recognized by at least one antigen receptor (e.g. a CAR or TCR) of the T cells.

Furthermore, the present invention provides a cell culture comprising
(i) a medium supplemented with a RHOG modulator that is suitable for suppressing, impairing or abolishing the expression and/or activity of RHOG in a T cell, as described herein and
(ii) a population of T cells, as described herein.

In addition, the present invention provides a method of culturing mammalian cells, said method comprising a step of culturing a population of T cells as described herein in a medium supplemented with a RHOG modulator that is suitable for suppressing, impairing or abolishing the expression and/or activity of RHOG in a T cell, as described herein.

In a further aspect, the invention provides a cell culture comprising T cells and a medium for culturing T cells according to the invention.

The invention also relates to method of culturing T cells, said method comprising a step of culturing a population of T cells in a medium for culturing T cells according to the invention.

In some preferred embodiments of a cell culture comprising T cells or a method of culturing T cells, the RHOG modulator comprises or is a RHOG inhibitor as described herein.

Furthermore, the present invention provides a method of producing at least one engineered mammalian cell, preferably at least one engineered T cell.

RHOG depletion or inactivation can be implemented broadly across T cell production platforms. In particular, it is compatible with existing and future electroporation devices (e.g., Lonza Amaxa platform, Maxcyte platform, Thermo Fisher's Neon platform and similar devices), T cell culture media (RPMI, ThermoFisher OpTmizer or similar media supplemented with a range of cytokines, e.g. IL-2, IL-7, IL-15) and a variety of T cell stimulation reagents such as anti-CD3/CD28, anti-CD2/CD3/CD28 stimulation beads, soluble antigen (recognized by the antigen receptor receptor), cells presenting the antigen recognized by the antigen receptor, antigen bound to beads, and antigen presented by lipid vesicles, are readily available and may be used in context of the present invention.

The present invention is broadly applicable across delivery systems for synthetic antigen receptors such a: retroviral vectors, lentiviral vectors, adenoviral vectors, adeno-associated viral vectors, transposomes, plasmid vectors with stable transmission during cell division (such as S/MAR vectors), standard mammalian expression vectors, RNPs and mRNA. In all applications involving synthetic CRISPR gRNAs or mRNAs, RNA modifications to maximize stability, minimize the immune response or have a beneficial impact on the editing outcome may be used. Such modifications include inter alia base modifications such as 5-methoxyuridine, phosphorothiate bonds, and/or 2′-OMe-rU, 2′-OMe-rC; see, e.g., Chiesa, N Engl J Med. 2023 Sep. 7; 389(10):899-910. Additionally, for applications involving mRNA a suitable capping structure, such as ARCA or CleanCap may be used. Furthermore, RHOG depletion or inactivation may be used in combination with closed cell production systems, such as the CliniMACS Prodigy platform (Miltenyi Biotec), and production in bioreactors used for T cell culture. RHOG depletion or inactivation according to the present invention is also compatible with the production of CAR T cells using microfluidics. Based on the data shown in the appended Examples, RHOG depletion or inactivation should result in production runs with higher numbers of therapeutic T cells, that have a more proliferative and metabolically activate phenotype and share features with memory T cells.

Accordingly, the present invention further relates to a method of producing at least one engineered mammalian cell, said method comprising:
a step (i) of contacting at least one mammalian cell with a RHOG modulator that is suitable for suppressing, impairing or abolishing the expression and/or activity of RHOG in a mammalian cell, in particular a T cell, as described herein and
prior to, simultaneously with and/or subsequently to said step (i),
a step (ii) of introducing a synthetic antigen receptor as described herein or at least one nucleic acid encoding such a synthetic antigen receptor into said mammalian cell(s). Preferably said synthetic antigen receptor is a CAR, as described herein. Preferably, at least one nucleic acid encoding a synthetic antigen receptor is introduced into the mammalian cell(s).

The present invention also relates to a method of producing at least one engineered mammalian cell, wherein said mammalian cell(s) comprise(s) a synthetic antigen receptor (preferably a CAR), as described herein, and wherein said method comprises a step of contacting said mammalian cell(s) with a RHOG modulator that is suitable for suppressing, impairing or abolishing the expression and/or activity of RHOG in a mammalian cell, in particular a T cell, as described herein.

The mammalian cell(s) may be (a) T cell(s), (a) T cell precursor cell(s), or (a) pluripotent stem cell(s). Preferably said pluripotent stem cell(s) is/are iPSCs. Preferably, the mammalian cell(s) comprise T cell(s), and further preferably, said mammalian cell(s) is/are cultured in a medium as described herein, e.g., in the context of the cell culture according to the invention.

Preferably, RHOG modulator used in the inventive method of producing at least one engineered mammalian cell is a RHOG modulator, as described herein above in context of the pharmaceutical composition according to the invention. In particular, the RHOG modulator may comprise a gene editing molecule and/or a gene editing modifier, an RNA interference (RNAi) molecule, and/or a DNA molecule for targeted integration into an endogenous RHOG gene, as described herein. More preferably, said RHOG modulator comprises a gene editing molecule and/or a gene editing modifier, as described herein. Herein and in context of the invention, contacting cells with a RHOG modulator may refer to introducing the RHOG modulator into the cells, in particular in the context of a gene editing molecule and/or a gene editing modifier, an RNA interference (RNAi) molecule, and/or a DNA molecule for targeted integration into an endogenous RHOG gene, as described herein. The RHOG modulator may also comprise a RHOG inhibitor as described herein, e.g., in context of in vivo administration or the inventive cell culture.

The mammalian cells may be contacted with the RHOG modulator according to the invention, e.g., a gene editing molecule and/or a gene editing modifier, by any suitable means as described herein and as known in the art. In particular, the RHOG modulator may be introduced into the mammalian cells, as described herein. Suitable means for this purpose include, inter alia, electroporation (e.g. of a RNP comprising a CRISPR system, at least one RNA such a gRNA and/or an mRNA), viral transduction, and transfection (e.g. of a transposon, a plasmid, at least one RNA such a gRNA and/or an mRNA, for example by lipofection). The same or similar means may be also used for introducing a synthetic antigen receptor into the mammalian cells, as described herein although a stable integration or long-term retention of the nucleic acid encoding the synthetic antigen receptor of the invention (e.g. by means of a viral vector, a transposon or a stably transmitted plasmid) is desirable. Further suitable means and methods are described herein, e.g., in context of the method for functional screening of engineered immune cells according to the invention, and in particular in context of “adding gene editing modifiers”. Electroporation and/or lipofection and/or the use of RNAs (such as gRNAs and/or mRNAs) and/or RNPs is/are particularly preferred in context of the present invention (especially in context of editing molecule and/or a gene editing modifier, because these means and methods are ideal for clinical uses; see also Example 3.

In particularly preferred embodiments an RNP comprising a CRISPR system as described herein is introduced into the mammalian cell(s), preferably by electroporation.

Engineered T cell precursor cells or pluripotent stem cells according to the invention may be further differentiated into T cells, e.g., by using protocols established in the prior art.

Accordingly, a population of engineered mammalian cells (e.g. T cells such as CAR T cells) according to the invention may be obtained by the inventive methods of producing at least one engineered mammalian cell described herein.

Furthermore, the present invention relates to an engineered mammalian cell comprising the following (I) and (II): (I)(a) a RHOG modulator that is suitable for suppressing, impairing or abolishing the expression and/or activity of RHOG in a mammalian cell, in particular, a T cell, as described herein, and/or (I)(b) an inactivating mutation in at least one allele of an endogenous RHOG gene, as described herein; and (II) a synthetic antigen receptor, preferably a chimeric antigen receptor (CAR), as described herein. In particular, the expression and/or activity of RHOG in said engineered mammalian cell is suppressed or abolished.

The engineered mammalian cell according to the invention may be a T cell, a T cell precursor cell, or a pluripotent stem cell. Preferably said pluripotent stem cell in an iPSC. Preferably, the engineered mammalian cell is a T cell according to the invention.

Furthermore, the present invention relates to a population of engineered mammalian cells comprising a plurality of the engineered mammalian cells according to the invention.

In addition, the present invention relates to a combination comprising a RHOG modulator as described herein and a T cell comprising a synthetic antigen receptor as described herein. The RHOG modulator and the T cell may be within the same composition or in separate compositions.

The present invention further provides a CRISPR guide RNA having a sequence selected from the group consisting of: SEQ ID NO: 5 to 26, preferably SEQ ID NO: 25.

The invention also relates to a combination comprising (I) at least one CRISPR guide RNA having a sequence selected from the group consisting of: SEQ ID NO: 19 to 26, and (II) a CRISPR endonuclease as described herein, or a nucleic acid encoding said CRISPR endonuclease.

In addition, the present invention relates to a combination comprising (I) at least one CRISPR guide RNA having a sequence selected from the group consisting of: SEQ ID NO: 5 to 18, and (II) a CRISPR base editor as described herein, or a nucleic acid encoding said CRISPR base editor.

Furthermore, the present invention also relates to a combination comprising (I) at least one pegRNA targeting a RHOG gene, and (II) a CRISPR prime editor as described herein, or a nucleic acid encoding said prime editor.

A combination of the invention may be for use in preventing, ameliorating or treating a disorder or disease, and/or for use in an immunotherapy, as described herein.

In addition, a combination of the invention may be used in a production method or a cell culture or cell culture method according to the invention.

The invention also relates to a kit comprising a combination according to the invention, and optionally a leaflet or brochure with instructions how to use the kit according to the invention.

The kit according to the invention may be for use in preventing, ameliorating or treating a disorder or disease, and/or for use in an immunotherapy, as described herein. Furthermore, the kit of the invention may be for use in a production method, cell culture and/or cell culture method, as described herein.

In some aspect, the invention provides a kit comprising at least one reagent for detecting an inactivating mutation in a RHOG gene, as described herein, and optionally a leaflet or brochure with instructions how to use the kit. Said kit may be used, e.g., for a method of producing a high-quality population of T cells for immunotherapy, a method of determining the quality of a population of T cells for immunotherapy as described herein, and/or a method of stratifying patients for T cell immunotherapy as described herein.

RHOG may also be used as a marker to select the starting material for T cell production, or to stratify patients and prioritize them for T cell immunotherapy.

Specifically, RHOG variants, and their interpretation based on the base editing data provided herein in context of the present invention, may be used to select the starting material (e.g. the initial T cells or iPSCs) to produce T cells for therapy, e.g. adoptive cell transfer. For example, the starting material can be analyzed with RHOG amplicon sequencing or similar methods, e.g., as described herein. Cells (e.g. T cells) with favorable RHOG variants associated with higher cell proliferation may be chosen as the starting material for production of therapeutic cells.

This concept applies broadly to the distinct types of starting material described herein.

Accordingly, the invention further relates to method of producing a high-quality population of T cells for immunotherapy, comprising the steps of:
(a) determining whether the RHOG gene in a population of mammalian cells, preferably T cells, comprises an inactivating mutation as described herein,
(b) selecting the population of cells when it comprises an inactivating mutation in the RHOG gene, and
(c) culturing the population of cells to produce a high quality population of T cells for immunotherapy.

Said mammalian cells may comprise, e.g., T cells, T cell precursor cells or iPSCs. Preferably, the population of T cells is cultured in a medium for culturing T cells, as described herein.

RHOG expression and/or activity levels may be further used to stratify patients and make informed treatment decisions. Herein, detailed methods are disclosed to measure RHOG inactivation, for example by amplicon sequencing of the RHOG gene. Further provided herein are results from saturating CRISPR base editing screens, as shown, e.g., in the appended Examples, which can be used to interpret the naturally occurring variants in RHOG within the patient population. For example, for each base edit, the corresponding z-score is provided which is a measure of T cell proliferation. Thus, sequencing of the RHOG locus could be used to prioritize patients for T cell based therapies.

Accordingly, the present invention further relates to a method of stratifying patients for T cell immunotherapy, comprising the steps of:
(a) determining whether the RHOG gene in cells, preferably T cells, from a patient comprises an inactivating mutation as described herein, and
(b) indicating that the patient is to be prioritized for T cell immunotherapy when the T cells from said patient comprises an inactivating mutation in the RHOG gene.

Preferably, said T cell immunotherapy may comprise adoptive transfer of autologous T cells.

RHOG may be also used as a marker for assessing the quality of a T cell product. Specifically, the expression and/or activity of RHOG may be profiled during quality control of a T cell product before treating patients. RHOG expression and/or activity can be assessed by a variety of methods known to the person skilled in the art, including, inter alia, amplicon sequencing, qPCR, immunofluorescence, and flow cytometry detecting RHOG. Based on the time course RNA-seq experiments provided herein in context of the present invention, for which the inventors compared RHOG KO versus SafeHarbor edited cells under repeated antigen stimulation, a gene signature can be derived from which individual genes or combinations can serve as quality control markers. For larger collections of genes, the use of bulk RNA-seq, single-cell sequencing, CyTOF, and similar techniques is envisioned.

Accordingly, the present invention further relates to a method of determining the quality of a population of T cells for immunotherapy, comprising the steps of:
(a) determining whether the RHOG gene in a population of T cells comprises an inactivating mutation as described herein, and
(b) determining that the population of T cells has a high quality when it comprises an inactivating mutation in the RHOG gene, and/or determining that the population of T cells has a low quality when it does not comprise an inactivating mutation in the RHOG gene.

Furthermore, the present invention relates to a method for functional screening of engineered immune cells, the method comprising the steps of:
(a) isolating immune cells from a sample;
(b) transducing the immune cells with a vector, the vector encoding a CAR and a gene editing molecule, preferably CRISPR guide RNA (gRNA);
(c) adding gene editing modifiers, preferably CRISPR modifiers to induce genetic modulation (e.g. CRISPR) editing;
(d) selecting a immune cell population based on a desired phenotype.

Accordingly, the invention also relates to a method for functional screening of engineered immune cells, the method comprising the steps of:
(a) isolating immune cells from a sample;
(b) transducing the immune cells with a vector, the vector encoding a CAR and a gene editing molecule, preferably CRISPR guide RNA (gRNA);
(c) adding gene editing modifiers, preferably CRISPR modifiers to induce genetic modulation (e.g. CRISPR) editing;
(d) selecting a immune cell population based on a desired phenotype.

Furthermore, the invention relates to a T cell population comprising a (lentiviral) vector, the vector encoding a CAR and a CRISPR guide RNA (gRNA).

The invention, in particular in context of the method for functional screening of engineered immune cells and the T cell population comprising a lentiviral vector, as described herein, provides, inter alia, further advantages as follows:
Combined expression of the CAR and gRNA cassette in one construct, ensuring that each functional CAR T cell is perturbed. Of note, this was not trivial to achieve since CAR-expressing vectors result in notoriously low lentiviral titers. In addition, the system of the present invention is compatible with the CROP-seq technology for single-cell CRISPR screens.
Presented here is the first example of a CRISPR screen done with mRNA technology.
Until now, genetic screens in human primary T cells have been limited to sorting readouts. In these screens, populations of cells can be actively selected, thus they tolerate low editing efficiencies and are technically less challenging. The present invention represents one of the first proliferation based CRISPR screens ever to be performed in human primary T cells²⁷. This type of screen can identify essential processes in CAR T cells, which can be enhanced by applying the opposite perturbation, e.g. by overexpression of transgenes.
Compared to state-of-the-art methods such as SLICE², the present invention can avoid recombinant protein production of the CRISPR modifier, e.g. SpCas9, but may instead use in vitro transcription (IVT) and electroporation of mRNA into cells. Recombinant protein expression and purification require extensive optimization for each CRISPR modifier, as well as specialized equipment and training. As the result, most labs outsource the production of recombinant SpCas9 protein to core facilities, or purchase expensive commercial products. In contrast, the present invention allows the production of virtually any CRISPR modifier using the exact same method, and requires only basic molecular biology skills and equipment. Moreover, co-delivery of mRNAs encoding a CRISPR modifier and antibiotic resistance gene is another distinguishing feature, which allows to select genome-edited cells produced by electroporation.
The method of the present invention avoids multiple rounds of lentiviral transduction, as commonly used even by world-leading labs^{3,4}. As the result, CAMEL (and the methods described herein) is much more efficient and yields a much greater number of T cells for the screen. This allows the user to screen at a higher gRNA complexity or to perform more readouts. Since the production of concentrated lentivirus is a major cost factor, CAMEL (and the herein described methods) is also cheaper than existing methods.
The present invention provides a fully optimized co-culture system of CAR T cells with target cells that allows us to isolate CAR T cells with interesting properties for immunotherapies. The present invention represents the first use of a trogocytosis readout for genetic screening in CAR T cells. Herein it is that the trogocytosis readout is a powerful on its own, but also a great filter for downstream screening strategies. The present invention distinguishes between intracellular (GFP) and extracellular (the antigen, e.g. CD19) markers of trogocytosis, allowing screens for fratricide-resistant CAR T cells.
The present invention provides an unexpected and straightforward solution to obtain enough CD8 T cells for genome-wide screening, through co-culture with CD4 helper cells.
The present invention demonstrates for the first time that a single integration of a CAR and gRNA encoding lentivirus is sufficient to control tumor growth in a mouse systemic leukemia model. Thus, the system of the present invention is suitable for pooled in vivo screening. This greatly reduces the workload and number of mice required for validation studies.
The present invention identifies a potential link between the gene ACSL5 and CAR T cell apoptosis due to overstimulation. Potentially, knockout of ACSL5 could render CAR T cells resistant to activation-induced cell death, a highly desirable property for immunotherapies.
The present invention for the first time performed proliferation-based screens in CAR T cells stimulated either via TCR or via CAR. From the comparison between these screens, the invention discovered a list of gene targets that can improve CAR T cell proliferation. This includes, among other hits, the unexpected finding that knockout of ZAP70 or LAT results in greater CAR T cell proliferation, likely due to reduced tonic signaling and T cell exhaustion. Another interesting hit is HAVCR2 (TIM3), which provides CAR T cells with the greatest expansion potential.
The invention includes a fully optimized laboratory pipeline for genetic screens in CAR T cells (that, inter alia, describes optimized T cell stimulation state, culture conditions and cell densities).
The number of gRNA integrations has so far not been adequately monitored for genetic screens in T cells. For the first time, the present invention provides exact measurements of the number of integrations, and was able to derive a highly reproducible method for the production and quantification of lentivirus, and the transduction of T cells.
The delivery of mRNA leads to only transient expression of the CRISPR modifiers, reducing immunogenicity and off target effects.
The method is particularly but not only suitable for modifications that are permanently installed, such as CRISPR knockout, base editing, mutagenesis or prime editing.

The present invention further established a workflow for pooled functional genetic screens in human CAR T cells, and for the first time allows the systematic discovery of genes that enhance CAR T cell function specifically or T cell function in general.

To enable pooled CRISPR screens, human primary T cells have to be modified with the following three components: (1) a chimeric antigen receptor (2) a library of CRISPR gRNAs, integrated into the genome to make perturbations genetically traceable (3) CRISPR editors that perform genome engineering, as directed by the gRNA. Once the cells are engineered, pooled screens rely on phenotypic readouts to physically separate cell populations of interest.

The present invention provides several advantages that result in a significantly improved workflow. Below, the current state of the art (regarding the genetic engineering of human primary T cells into CAR T cells and for installing CRISPR edits, functional assays to measure CAR T cell phenotypes and performance, list of published genome-wide CRISPR screens in human T cells) is summarized over which the present invention provides advantages.

Lentivirus transduction is a standard method for the stable expression and permanent integration of genetic constructs into the genome. It is widely used for immunotherapy manufacturing and CRISPR applications in T cells.

In the context of T cell based immunotherapies, lentiviral vectors are used to deliver synthetic immune receptors that enable the recognition of specific antigens, for example a CAR¹⁵ or TCR¹⁶. Various protocols for lentivirus transduction of human primary T cells have been described, and sequences of CAR building blocks have been reported previously¹⁷. In the context of pooled genetic screens, CRISPR modifiers are usually delivered via the electroporation of Cas9 protein (SLICE method^{2,4}) or by two successive transductions with different lentiviral constructs, where one encodes a CRISPR modifier, and the other a gRNA library³. However, inefficiencies in the delivery of each lentivirus result in low numbers of double-positive cells and multiple rounds of lentivirus transduction yield heterogenous expression levels for the different constructs. In addition, the production of two separate lentivirus preparations increases the experimental costs. The present invention represents the first co-delivery of a CAR and gRNA library on a single vector, thereby providing an important increase the efficiency and scalability of CRISPR screens for optimizing CAR T cells. In other words, the inventors surprisingly and unexpectedly found that a vector can be used that encodes both the CAR and the gRNA. Of note, it is not trivial to use a system where two separate vectors encoding CAR and gRNAs are used due to low lentiviral titers and low transduction rates in primary cells.

Co-culture with target cells is a popular way to assess CAR T cell phenotypes and performance. In such co-culture systems, the phenomenon of trogocytosis has been observed by flow cytometry. For example, CAR T cells stimulated with target cells expressing a CD19-mCherry fusion protein acquired both mCherry and CD19 as part of their activation⁵. However, trogocytosis has not been used or suggested as a screening readout to select active CAR T cells or to optimize them. The present inventors surprisingly and unexpectedly found that trogocytosis can be used as a screening marker, alone or as a filter for existing FACS readouts to pre-select functional CAR T cells.

Most notably, the present invention enables entirely new screening readouts, such as receptor internalization versus presentation on the CAR T cell surface. Functional assessment of CAR T cells routinely includes preclinical mouse models, where CAR T cells are injected into tumor-bearing mice, and the tumor clearance is monitored. In principle, mouse models are compatible with direct cytotoxicity assessment, as well as proliferation-, FACS-, or single-cell based screens. However, no in vivo screens were performed with human T cells. The present inventors found that the method of the invention supports in vivo screens, including experiments based on CROP-seq¹ for single-cell readouts.

Existing strategies for pooled genetic screens can be classified into three directions¹⁸. First, proliferation-based screens identify genetic perturbations that affect cell fitness based on the enrichment or depletion of cells with particular gRNAs over time, for example during cell culture in vitro. Second, FACS-based screens physically separate cells based on the expression of certain markers and extend CRISPR screens to more complex phenotypes. Third, single-cell CRISPR screens combine pooled screening with a single-cell transcriptome^{1,19-21} or epigenome readout^{22,23}. Below current readout strategies for pooled CRISPR screens in human primary T cells are reviewed over which the present invention provides advantages.

Shifrut and colleagues first demonstrated genome-wide screens in CD8+ human primary T cells using delivery of the gRNA library by lentiviral transduction and electroporation of Cas9 protein (the SLICE method²). The resulting genome-edited T cells were stimulated with anti-CD3/CD28 beads, and cell divisions were tracked by cell staining with carboxyfluorescein succinimidyl ester (CFSE). FACS was used to isolate cells with high and low proliferation rates.

In addition to loss-of-function screens, Schmidt and colleagues demonstrated the possibility of CRISPR activation and interference screens in CD4+ and CD8+ T cells³. CRISPR modifiers and gRNA libraries were introduced one-by-one using different lentiviral constructs. The authors used FACS to separate CD4+ or CD8+ T cells with high and low levels of IFNγ or IL2, to identify genetic perturbations that affect cytokine production.

Only a single paper reports a genome-wide CRISPR screen in CAR T cells⁴. CAR and gRNA library were separately introduced with two different lentiviral constructs, and Cas9 was delivered by protein electroporation. CAR T cells were then repeatedly stimulated with target cells, inducing expression of the T cell exhaustion marker PD1 in a subset of cells. T cells were FACS-sorted into PD1-negative and PD1-positive cells, and the gRNA content of these populations was compared to identify regulators of PD1 expression and T cell exhaustion. The fact that gRNAs targeting PD1 were not enriched in the PD1-negative population indicates that the screen suffers from insufficient genome editing and high noise.

Taken together, only FACS-based but no proliferation-based CRISPR screens have been performed in primary human T cells, and there is a single report of FACS-based CRISPR screening in human CAR T cells. The present invention provides a superior approach for manufacturing genome edited CAR T cells in a pooled setting, includes advanced FACS-based screens with pre-selection of active CAR T cells, and for the first time enables proliferation-based CRISPR screens in human primary CAR T cells.

The invention further describes a laboratory pipeline for functional genetic screens in human chimeric antigen receptor (CAR) T cells. It is based on and applies the CAMEL technology (FIG. 1a) for effective CRISPR editing to primary human T cells, to optimize them for immunotherapy applications.

First, T cells are isolated from human peripheral blood (typically blood donations from healthy volunteers but potentially also blood from cancer patients), then cultured and stimulated under defined conditions. While undergoing an optimized culture and stimulation regimen, they are first transduced with a lentiviral vector to express a chimeric antigen receptor (CAR) and CRISPR guide RNA (gRNA) cassette. It is particularly preferred that the CAR and gRNA are encoded on one vector. Later, CRISPR modifiers are delivered as in vitro transcribed messenger RNA (mRNA), resulting in efficient CRISPR editing. Selection with antibiotics or by fluorescence-activated cell sorting (FACS) is used to enrich a pool of modified T cells.

To test the performance of T cells, the invention provides a co-culture system with engineered target cells. Using this system of the invention, CAR T cells can effectively be isolated based on their cell type (CD4, CD8), engagement with target cells (CD19, GFP), T cell activation (CD69), degranulation (CD107a), cytokine production (IFNg), the expression of death receptors (Fas), T cell fratricide (culture over time, Fas, ratio between CD19 and GFP), or T cell exhaustion (PD1). Importantly, the invention provides combinations of these markers into novel multi-step sorting strategies to isolate CAR T cells with desirable properties. To process the resulting material, the invention provides protocols for cell fixation, de-crosslinking for efficient genomic DNA (gDNA) isolation, single-step gRNA amplification, and next-generation sequencing. Finally, the invention provides genome-wide screens with proliferation and sorting readout, demonstrating that the means and methods provided by the invention is highly scalable and efficient. The invention also provides in vivo screens in mouse tumor models.

The above is illustrated in the appended examples.

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, the preferred materials and methods are described herein.

It is also to be understood that the terminology used herein is for the purpose of describing particular embodiments only, and is not intended to be limiting.

The articles “a” and “an” may be used herein to refer to one or to more than one (i.e., to at least one) of the grammatical object of the article. By way of example, “an element” means one element or more than one element.

It is envisaged herein that the method of the present invention is used for functional screening of engineered cells.

It is particularly preferred that the transgene and the gRNA are encoded by the same vector because it provides the herein described advantages for example that each functional CAR T cell is also transduced/transfected with a gRNA and, thus, perturbed.

Accordingly the invention relates to a method for functional screening of engineered immune cells, the method comprising the steps of:
(a) isolating cells from a sample
(b) transducing the cells with a vector encoding a transgene and a gene editing molecule, preferably CRISPR guide RNA (gRNA);
(c) adding gene editing modifiers, preferably CRISPR modifiers to induce genetic modulation (e.g. CRISPR editing);
(d) selecting a cell population based on desired phenotype.

Preferably the cell is an immune cell. Preferably the immune cell is a T cell. Preferably, the T cell is a CTL or a CAR T cell.

Preferably, the cell is transduced with a vector comprising a CAR in addition to the gene editing molecule.

It is envisaged that the cell is an immune cell. Accordingly, the invention relates to a method for functional screening of engineered immune cells, the method comprising the steps of:
(a) isolating immune cells from a sample;
(b) transducing the immune cells with a vector encoding a transgene and a gene editing molecule, preferably CRISPR guide RNA (gRNA);
(c) adding gene editing modifiers, preferably CRISPR modifiers to induce genetic modulation (e.g. CRISPR editing);
(d) selecting a immune cell population based on desired phenotype.

It is also envisaged herein that the cell is a T cell. Accordingly, the invention relates to a method for functional screening of engineered T cells, the method comprising the steps of:
(a) isolating T cells from a sample;
(b) transducing the T cells with a vector encoding a transgene a gene editing molecule, preferably CRISPR guide RNA (gRNA);
(c) adding gene editing modifiers, preferably CRISPR modifiers to induce genetic modulation (e.g. CRISPR editing);
(d) selecting a T cell population based on desired phenotype.

It is also envisaged herein that the cell is a CTL. Accordingly, the invention relates to a method for functional screening of CTLs, the method comprising the steps of:
(a) isolating CTLs from a sample;
(b) transducing the CTLs with a vector encoding a transgene and a gene editing molecule, preferably CRISPR guide RNA (gRNA);
(c) adding gene editing modifiers, preferably CRISPR modifiers to induce genetic modulation (e.g. CRISPR editing);
(d) selecting a CTL population based on desired phenotype.

It is further envisaged herein that the cell is preferably a CAR T cell. Accordingly, the invention relates to a method for functional screening of CAR T cells, the method comprising the steps of:
(a) isolating T cells from a sample;
(b) transducing the T cells with a vector, the vector encoding a CAR and a gene editing molecule, preferably CRISPR guide RNA (gRNA);
(c) adding gene editing modifiers, preferably CRISPR modifiers to induce genetic modulation (e.g. CRISPR editing);
(d) selecting a CAR T cell population based on desired phenotype.

The sample as used herein can be every source from which cells suitable for the herein described methods can be derived/isolated. It is envisaged herein that in the methods described herein the sample is a blood sample. The blood sample may be obtained from a healthy donor or a patient, preferably cancer patient. Preferably the blood sample is a peripheral blood sample.

Accordingly, the invention relates to a method for functional screening of CAR T cells, the method comprising the steps of:
(a) isolating T cells from a blood sample;
(b) transducing the T cells with a vector, the vector encoding a CAR and a CRISPR guide RNA (gRNA);
(c) adding gene editing modifiers, preferably CRISPR modifiers to induce genetic modulation (e.g. CRISPR editing);
(d) selecting a CAR T cell population based on desired phenotype.

Accordingly, the invention relates to a method for functional screening of CAR T cells, the method comprising the steps of:
(a) isolating T cells from a peripheral blood sample;
(b) transducing the T cells with a vector, the vector encoding a CAR and a CRISPR guide RNA (gRNA);
(c) adding gene editing modifiers, preferably CRISPR modifiers to induce genetic modulation (e.g. CRISPR editing);
(d) selecting a CAR T cell population based on desired phenotype.

The term “adding gene editing modifiers” is used herein in the broadest sense and refers to all techniques that bring the gene editing modifier into contact with the DNA of the cell or organism, preferably together with the gene editing molecule. For example, a polynucleotide (e.g. DNA or RNA) encoding said gene editing modifier or a protein like in SLICE is transduced/transfected/transformed into the cell. Preferably, the polynucleotide is RNA, in particular mRNA. Accordingly, it is envisaged that in the methods described herein the CRISPR modifiers are added in the form of RNA, in particular mRNA.

It is preferred herein that the gene editing modifier are added via electroporation. Accordingly, it is preferred herein that the CRISPR modifiers are introduced via electroporation.

The vector as used in the herein described methods may be selected from any molecule that is suitable to transport e.g. the CAR and the gRNA into the cell. The vector may be lentiviral vector or a sleeping Beauty (SB) transposon, preferably a lentiviral vector.

The skilled person is well aware of the techniques with respect to Sleeping Beauty (SB) transposons (see e.g. references 25 and 26).

The invention provides a series of lentiviral transfer plasmids encoding a chimeric antigen receptor (CAR) and CRISPR guide RNA (gRNA) expression cassette, which are, inter alia, part of the invention and enable powerful and effective optimization screens.

Accordingly, it is envisaged herein that the vector is preferably a lentiviral vector. Accordingly, the invention relates to a method for functional screening of CAR T cells, the method comprising the steps of:
(a) isolating T cells from a sample, preferably a (peripheral) blood sample;
(b) transducing the T cells with a lentiviral vector, the lentiviral vector encoding a CAR and a gene editing molecule, preferably CRISPR guide RNA (gRNA);
(c) adding gene editing modifiers, preferably CRISPR modifiers to induce genetic modulation (e.g. CRISPR editing);
(d) selecting a CAR T cell population based on desired phenotype.

Preferably, lentiviral vector configuration enables single-cell sequencing.

However, it is also envisaged that the vector is a sleeping Beauty transposon. Accordingly, the invention relates to a method for functional screening of CAR T cells, the method comprising the steps of:
(a) isolating T cells from a sample, preferably a (peripheral) blood sample;
(b) transducing the T cells with a sleeping Beauty transposon, the sleeping Beauty transposon encoding a CAR and a gene editing molecule, preferably CRISPR guide RNA (gRNA);
(c) adding gene editing modifiers, preferably CRISPR modifiers to induce genetic modulation (e.g. CRISPR editing);
(d) selecting a CAR T cell population based on desired phenotype.

The invention also relates to a vector encoding a CAR and a gene editing molecule. The gene editing molecule is preferably a CRISPR guide RNA (gRNA).

The lentiviral vector as used in the herein described methods may comprise the gRNA at the 3′ long terminal repeat (LTR).

The lentiviral vector as used in the herein described methods may comprise the CAR 5′ of the gRNA.

The CAR as used in the herein described methods may be expressed under the EF1α or the CMV promoter, preferably under the EF1α promoter.

The features described herein in connection with a lentiviral vector may also be used with other vectors as applicable.

In the methods described in context of the present invention wherein the CAR may comprise a leader peptide and a single-chain variable fragment (scFv) antibody, the CAR may further comprise the hinge- and transmembrane domains of CD8, the intracellular co-stimulatory domains of CD28 and/or 41BB and/or the CD3ζ chain.

Accordingly, the invention relates to a method for functional screening of CAR T cells, the method comprising the steps of:
(a) isolating T cells from a sample, preferably a blood sample, more preferably a peripheral blood sample;
(b) transducing the T cells with a lentiviral vector, the lentiviral vector encoding a CAR and a gene editing molecule, preferably CRISPR guide RNA (gRNA), wherein the CAR comprises a leader peptide, a single-chain variable fragment (scFv) antibody and optionally the hinge- and transmembrane domains of CD8, the intracellular co-stimulatory domains of CD28 and/or 41BB and/or the CD3ζ chain;
(c) adding gene editing modifiers, preferably CRISPR modifiers to induce genetic modulation (e.g. CRISPR editing);
(d) selecting a CAR T cell population based on desired phenotype.

It is envisaged herein that the single-chain variable fragment (scFv) antibody of the CAR used in context of the herein described methods is specific for CD19, BCMA, GD2, HER2, CD123, CD138, CD20, CD22, CD38, CDK5, IgK, LeY, NKG2D, ROR1, WT1, c-met, CAIX, CD70, CEA, EGFR, EpCAM, EphA2, FAP, GD2, HER2, IL13RA2, LeY, MAGEA3, MAGEA4, MART1, Mesothelin, MUC1, MUC16, NY-ESO-1, PD-L1, PSCA, PSMA, or VEGFR2, preferably CD19.

Accordingly, the invention relates to a method for functional screening of CAR T cells, the method comprising the steps of:
(a) isolating T cells from a sample, preferably a blood sample, more preferably a peripheral blood sample;
(b) transducing the T cells with a lentiviral vector, the lentiviral vector encoding a CAR and a gene editing molecule, preferably CRISPR guide RNA (gRNA), wherein the CAR comprises a leader peptide, a single-chain variable fragment (scFv) antibody specific for CD19 and optionally the hinge- and transmembrane domains of CD8, the intracellular co-stimulatory domains of CD28 and/or 41BB and/or the CD3ζ chain;
(c) adding gene editing modifiers, preferably CRISPR modifiers to induce genetic modulation (e.g. CRISPR editing);
(d) selecting a CAR T cell population based on desired phenotype.

A CAR T cell according to the invention may be generated as described herein, e.g., as illustrated in Example 3. It is preferred in context of the present invention that a protocol is used which is compatible with or optimized for clinical uses, as described herein. Furthermore, a CAR T cell according to the invention may be generated as follows:

The gRNA is cloned into the 3′ LTR, making the system compatible with the CROP-seq technology for screens in single cells¹. CARs are expressed under the EF1α promoter, and composed of a leader peptide, single-chain variable fragment (scFv) antibody binding CD19 (clone FMC63 or SJ25C1), optionally the hinge and transmembrane domains of CD8, intracellular co-stimulatory domains (CD28 or 41BB) and the CD3ζ chain. In total 21 such plasmids are provided, using different combinations of these building blocks. A preferred plasmid is pD0039 (SEQ ID NO:1 and FIG. 15) Upon successful delivery of the constructs, T cells express the chimeric antigen receptor on their surface (FIG. 2a), and mediate cytotoxic killing of CD19-expressing tumor cell lines in vitro and control tumor growth in a systemic leukemia model as described herein.

The plasmid design of the present invention has, inter alia, one major advantage: for the first time the CAR and CRISPR gRNA are combined on the same vector. This turned out to be a crucial design decision for efficient screening, since it ensures that each functional CAR T cell also contains a CRISPR perturbation. This approach is surprisingly and unexpectedly much more efficient than using multiple lentiviruses in succession, as for example done in 3 and 4. Using CAMEL, a much higher number of T cells can be modified, which allows higher quality screens with larger libraries, deeper coverage, and/or multiple functional readouts.

The present invention provides versions of the above described plasmids that encode CRISPR gRNA libraries. These libraries were PCR-amplified from custom-designed oligo pools or existing plasmid libraries, and cloned by Gibson's isothermal assembly. They include small-scale libraries targeting selected control genes, libraries targeting curated T cell genes, a small-scale base editing library and genome-wide libraries targeting each human gene with four gRNAs. In addition, single gRNA expressing constructs and established flow cytometric assays for genome editing of the surface markers CD25 and CD44 are provided which can be used for protocol development and to monitor the editing efficiency during the screen.

The efficiency of T cell modification depends on their stimulation status, the culture conditions and their surface density. The present invention provides a fully optimized, scalable, and highly efficient laboratory workflow for genetic screens in CAR T cells. A typical screen may follow the experimental timeline in FIG. 3. Human primary CD4+ or CD3+ T cells may be isolated from peripheral blood donations by negative magnetic selection. Immediately upon isolation, T cells may be activated by TCR stimulation and co-stimulation via anti-CD3/anti-CD28 activator reagents. T cells may be cultured in OpTmizer medium with 2% human serum and supplemented with IL2. To obtain sufficient cells for genome-wide screening, cells can undergo an initial expansion period of 9-10 days, and may then be re-stimulated. On day 2 post stimulation, cells may be transduced with a defined amount of highly concentrated lentivirus encoding the CAR and one or more gRNAs or a gRNA library. On day 4 post stimulation, cells may be electroporated with custom-made mRNA encoding a CRISPR modifier and selection marker (e.g., encoding BSD for blasticidin resistance). This step initiates the genome editing process. At day 5 or 6 post stimulation, cells may be selected with both puromycin (to select for the CAR/gRNA construct) and blasticidin (to select for successfully electroporated cells). The puromycin selection may then be maintained until the end of the experiment. Functional readouts are ideally done on days 5-6 post electroporation, to allow for efficient cell engineering and selection of modified cells.

The inventors intensely studied T cell transduction. This has allowed the inventors to derive methods for the titration and exact transduction of human primary T cells, which result in consistent transduction rates across many lentivirus production rounds and T cell donors.

Importantly, the optimization data of the present invention shows that in a culture of primary human T cells, only a subpopulation of cells is amenable to lentivirus transduction. This is demonstrated by T cell transduction with high lentivirus concentrations, where a 10-fold difference between virus volumes does not lead to increased transduction efficiency (FIG. 4a). One likely explanation is that only a fraction of cells is fully activated by the stimulation beads, leading to proliferation and changes in their membrane composition that facilitate viral entry. This hypothesis is supported by varying transduction efficiencies in cells treated with different TCR stimulation agents, among which ImmunoCult CD3/CD28 Activator (STEMCELL Technologies) led to the highest transduction rates and therefore was chosen for genetic screens (FIG. 4a). Accordingly, in context of the present invention the TCR stimulation agents ImmunoCult CD3/CD28 Activator (STEMCELL Technologies) is preferred.

In the process of further optimization of transduction conditions, the inventors found that commonly used reagents such as polybrene are toxic to T cells (FIG. 4b) and that frequently used methods such as spinfection and plate coating with retronectin do not further improve T cell transduction (FIG. 4c). Furthermore, the inventors showed that transduction is optimal on day 3 for CD8 cells (FIG. 4c). Accordingly, in context of the present invention it is preferred that CD8 cells are transduced on day 3.

Transduction rates in T cells saturate in the range of 10-50% (FIG. 4a), but increasing lentivirus amounts may introduce multiple copies of the construct. In a CRISPR screen with a gRNA library, each cell should ideally receive only one gRNA. Multiple integrations would result in a random set of genetic perturbations per cell. The herein described method quantifies the lentivirus titer with a qPCR-based assay, which reproducibly identifies the number of lentivirus genome copies per volume unit (FIG. 4d). Then a standardized number of virus copies per cell may be used (247 copies per cell for screens in CD4 T cells and 432 copies per cell for screens in CD8 T cells). This typically results in transduction rates of ~15% (CD4) or ~10% (CD8) (FIG. 4e). By expansion of single cell clones followed by gRNA sequencing, it is shown that this approach results in an average number of 1.6 gRNA integrations per successfully transduced cell (FIG. 4f). In addition, the inventors verified the number of integrations with digital droplet PCR (ddPCR) that simplifies the verification of integration number in antibiotic-selected cells and correlates well with gRNA sequencing-based counting of integrations in single cell expanded clones (FIG. 4g).

In a genome-wide screen, most perturbations will not have an effect on the phenotype. Thus, it is preferred herein to choose a concentration where some cells still have double integrations, to maximize the number of T cells that are transduced for the screen. Accordingly, it is preferred in context of the present invention that 247 virus copies per cell for screens in CD4 T cells and 432 virus copies per cell for screens in CD8 T cells are used.

Large cell numbers are required to maintain high gRNA coverage at every step of a genetic screen. To obtain maximum T cell numbers, the present inventors extensively optimized cell culture conditions. IL2 is the major supplement that supports T cell growth. The inventors found optimal concentrations for two IL2 reagents (FIG. 5a). Accordingly, in context of the present invention Stemcell IL2 may be used at 12.1 ng/ml.

It is shown that the IL2 addition is sufficient for proliferation, while other frequently used supplements, such as IL7, IL15, IL21, beta-mercaptoethanol (b-ME) did not further improve T cell expansion (FIG. 5b).

The inventors surprisingly and unexpectedly found that CD8 T cells do not sufficiently expand for genome-wide screens when cultured alone, but proliferate well in the presence of CD4 helper cells (FIG. 5b). Thus, the present invention describes a co-culture method for effective proliferation of CD8 T cells in genome-wide CRISPR screens. Accordingly, the present invention relates to a method for effective proliferation of CD8 T cells, wherein the CD8 T cells are co-cultured with CD4 helper cells.

The inventors also surprisingly and unexpectedly found that CD4 cells alone have a much greater proliferation potential than in mixture with CD8 cells, where the CD4:CD8 ratio declines over time (FIG. 5c). Thus, the present invention demonstrates for the first time that genome-wide screens in CD4 cells are only feasible when cultured alone, and that screens in CD8 cells require the isolation of pan-CD3 cells, where CD8 cells will eventually grow out.

Accordingly, the present invention relates to a method for effective proliferation of CD4 T cells, wherein the CD4 T cells are cultured alone (in the absence of CD8 T cells).

The inventors also observed that the density of T cells and CAR T cells on the surface of culture vessels has an influence on their transduction, electroporation, and target cell killing.

Furthermore, for genetic screens, the inventors determined antibiotic concentrations to select for successfully transduced and electroporated cells. A puromycin concentration of 0.5 μg/ml leads to >75% death of untransduced cells within 1 week of selection (FIG. 6a), and >90% selection of CAR-positive cells within 10 days (FIG. 6b). Accordingly, in context of the herein described inventive methods a puromycin concentration of 0.5 μg/ml may be used. Depending on the timepoint of the readout, a minor fraction of untransduced cells may remain in the culture. However, this will not affect the pooled screening results, as untransduced cells do not contain a gRNA. Efficient antibiotic selection is nevertheless critical to minimize cell culture expenses and is generally superior to FACS, which is more damaging to cells and limiting for large screens. At the same time, selection of electroporated cells can substantially improve screening results by removing cells that did not express a CRISPR modifier and therefore did not receive a genetic modification. To select edited cells, the methods described in context of the present invention may be used with co-electroporation of e.g. BSD mRNA together with e.g. CRISPR editor mRNA. One day post electroporation, cells may be treated with blasticidin at a concentration of 50 μg/ml for a duration of 1-2 days, which leads to >85% death of non-electroporated cells (FIG. 6c). Using this approach, 75-90% editing can be achieved on two different electroporators (Amaxa Nucleofector I and MaxCyte ATX) with optimized programs (FIG. 6d). Accordingly, in context of the present invention when the gene editing modifier is co-transduced with e.g. BSD mRNA one day post electroporation, cells may be treated with blasticidin at a concentration of 50 μg/ml for a duration of 1-2 days.

It is envisaged herein that the engineered/modified (immune) cells are contacted/co-cultured with a second cell population to e.g. activate the engineered/modified (immune) cells and then to screen the engineered/modified (immune) cells for desired properties/characteristics (e.g. as indicated by a biological phenotype).

The key to a successful screen is an efficient way to isolate populations of interest from a pool of modified cells. The gRNAs in each population can then be counted by next-generation sequencing, to find CRISPR perturbations that are enriched or depleted in biological phenotypes. To isolate CAR T cells with desirable properties for immunotherapies, the present invention provides an innovative co-culture system with target cells. For this system, the invention provides engineered cancer cell lines that may express the antigen recognized by the CAR on their surface (e.g. human CD19), in fusion with a reporter gene such as GFP. As a negative control, target cells may instead be engineered with an unspecific antigen not bound by the CAR (such as human CD20) that is fused to the same reporter.

As described in detail in FIG. 7 and FIG. 8, when functional T cells engage with their target, the T cell receptor (TCR) pathway is activated and the TCR gets internalized. This also results in the uptake of the antigen and parts of the target cell membrane by T cells, a process known as trogocytosis. The present inventors found that both the antigen and reporter are readily detected on the surface and inside of functional CAR T cells when they engage with their targets. The invention establishes for the first time that trogocytosis can be effectively used as a screening readout, using e.g GFP or CD19 staining as reporter. This readout can be used alone, to identify CAR T cells that have successfully engaged with their targets. However, it is just as powerful when used as a filter for downstream readouts that are provided herein. For example, CAR T cells may be isolated that have successfully recognized their target, but do not show any downstream signaling (CD69), degranulation (CD107a), or cytokine production (IFNγ) (FIG. 7).

Accordingly, the invention relates to a method for functional screening of CAR T cells, the method comprising the steps of:
(a) isolating T cells from a sample, preferably a (peripheral) blood sample;
(b) transducing the T cells with a vector, the vector encoding a CAR and a gene editing molecule, preferably CRISPR guide RNA (gRNA);
(c) adding gene editing modifiers, preferably CRISPR modifiers to induce genetic modulation (e.g. CRISPR editing);
(d) selecting a CAR T cell population based on desired phenotype;
wherein the CAR T cells are co-cultured with a second cell population.

As also described herein it is envisaged that the second cell population is engineered to express one or more reporter construct(s).

Accordingly, the invention relates to a method for functional screening of CAR T cells, the method comprising the steps of:
(a) isolating T cells from a sample, preferably a (peripheral) blood sample;
(b) transducing the T cells with a vector, the vector encoding a CAR and a gene editing molecule, preferably CRISPR guide RNA (gRNA);
(c) adding gene editing modifiers, preferably CRISPR modifiers to induce genetic modulation (e.g. CRISPR editing);
(d) selecting a CAR T cell population based on desired phenotype;
wherein the CAR T cells are co-cultured with a second cell population and wherein the second cell population is engineered to express one or more reporter construct(s).

As already mentioned said reporter construct may be detected in or on the engineered (immune) cell to screen whether the engineered (immune) cell (preferably CAR T cell) has engaged with the cells of the second cell population. In other words, the said reporter may be detected in or on the engineered (immune) cell to screen whether the engineered (immune) cell has engaged with the cells of the second cell population. Thus, the detection of the reporter construct on the surface or inside the engineered (immune) cell is indicative of engagement of the engineered (immune) cell with the second cell population.

Accordingly, the invention relates to a method for functional screening of CAR T cells, the method comprising the steps of:
(a) isolating T cells from a sample;
(b) transducing the T cells with a vector, the vector encoding a CAR and a gene editing molecule, preferably CRISPR guide RNA (gRNA);
(c) adding gene editing modifiers, preferably CRISPR modifiers to induce genetic modulation (e.g. CRISPR editing);
(d) selecting a CAR T cell population based on desired phenotype;
wherein the CAR T cells are co-cultured with a second cell population, wherein the second cell population is engineered to express one or more reporter construct(s);
and wherein detection of the reporter construct on the surface or inside the T cell is indicative of engagement of the T cell with the second cell population.

It is envisaged herein that the reporter construct used in the herein described methods comprises an antigen and/or a reporter. Said antigen may be recognized/targeted/bound by the engineered (immune) cell. It is envisaged that the antigen used for the reporter is an antigen selected from the group consisting of CD19, BCMA, GD2, HER2, CD123, CD138, CD20, CD22, CD38, CDK5, IgK, LeY, NKG2D, ROR1, WT1, c-met, CAIX, CD70, CEA, EGFR, EpCAM, EphA2, FAP, GD2, HER2, IL13RA2, LeY, MAGEA3, MAGEA4, MART1, Mesothelin, MUC1, MUC16, NY-ESO-1, PD-L1, PSCA, PSMA, and VEGFR2 and parts or fragments thereof. It is preferred that the antigen is CD19 or a fragment thereof.

It is envisaged herein that the reporter is selected form the group consisting of GFP, BFP and mCherry. Preferably, the reporter gene is GFP. The skilled person is well aware of other suitable reporters and may choose them as required.

Of course, it is possible that the reporter construct comprises only an antigen and no reporter, It is also envisaged that the reporter construct consists of an antigen. It is also envisaged that the reporter construct is a herein described antigen and does not comprise a reporter. A reporter construct comprising only an antigen may be particularly suitable when the second cell population is needed to only stimulate the engineered immune cell and it is not intended to detect the reporter construct in or on the engineered immune cell.

Accordingly, the invention relates to a method for functional screening of CAR T cells, the method comprising the steps of:
(a) isolating T cells from a sample, preferably a blood sample, more preferably a peripheral blood sample;
(b) transducing the T cells with a vector, the vector encoding a CAR and a gene editing molecule, preferably CRISPR guide RNA (gRNA);
(c) adding gene editing modifiers, preferably CRISPR modifiers to induce genetic modulation (e.g. CRISPR editing);
(d) selecting a CAR T cell population based on desired phenotype;
wherein the CAR T cells are co-cultured with a second cell population, wherein the second cell population is engineered to express one or more reporter construct(s) and wherein the reporter construct comprises and antigen and a reporter.

The invention also relates to a method for functional screening of CAR T cells, the method comprising the steps of:
(a) isolating T cells from a sample, preferably a blood sample, more preferably a peripheral blood sample;
(b) transducing the T cells with a vector, the vector encoding a CAR and a gene editing molecule, preferably CRISPR guide RNA (gRNA);
(c) adding gene editing modifiers, preferably CRISPR modifiers to induce genetic modulation (e.g. CRISPR editing);
(d) selecting a CAR T cell population based on desired phenotype;
wherein the CAR T cells are co-cultured with a second cell population, wherein the second cell population is engineered to express one or more reporter construct(s), wherein the reporter construct comprises and antigen and a reporter and wherein the reporter is GFP.

The invention also relates to a method for functional screening of CAR T cells, the method comprising the steps of:
(a) isolating T cells from a sample, preferably a blood sample, more preferably a peripheral blood sample;
(b) transducing the T cells with a vector, the vector encoding a CAR specific for CD19 and a gene editing molecule, preferably CRISPR guide RNA (gRNA);
(c) adding gene editing modifiers, preferably CRISPR modifiers to induce genetic modulation (e.g. CRISPR editing);
(d) selecting a CAR T cell population based on desired phenotype;
wherein the CAR T cells are co-cultured with a second cell population, wherein the second cell population is engineered to express one or more reporter construct(s), wherein the reporter comprises an antigen and a reporter and wherein the antigen is CD19.

In a preferred embodiment, the invention also relates to a method for functional screening of CAR T cells, the method comprising the steps of:
(a) isolating T cells from a sample, preferably a blood sample, more preferably a peripheral blood sample;
(b) transducing the T cells with a vector, the vector encoding a CAR specific for CD19 and a gene editing molecule, preferably CRISPR guide RNA (gRNA);
(c) adding gene editing modifiers, preferably CRISPR modifiers to induce genetic modulation (e.g. CRISPR editing);
(d) selecting a CAR T cell population based on desired phenotype;
wherein the CAR T cells are co-cultured with a second cell population, wherein the second cell population is engineered to express one or more reporter construct(s), wherein the reporter construct comprises and antigen and a reporter, wherein the antigen is CD19 and the reporter is GFP.

To functionally test CAR T cells both in vitro and in vivo in context of the methods of the present invention, a number of plasmid constructs are provided herein to establish stable cell lines and functional assays. To support bioluminescence imaging, the invention provides a transgene of firefly luciferase, the resistance gene for the antibiotic zeocin and the fluorescent reporter mPlum. These constructs may be used to establish stable lines from the B cell precursor leukemia cell line NALM6 that expresses endogenous levels of CD19. The invention provides a plate-based high-throughput assays of CAR T cell killing activity based on firefly luciferase (FIG. 2b) and a systemic leukemia model in NSG mice as described elsewhere herein. To further investigate the effect of surface antigen density, CRISPR may be used to induce indels in the CD19 coding sequence, and isolated heterozygous mutants by clonal expansion. The resulting cell lines may show reduced expression level of CD19 (FIG. 2c) and may be used to test CAR T cells in the context of antigen downregulation.

Furthermore, the invention provides constructs encoding the antigen recognized by the CAR (e.g., CD19) or an unspecific negative control (e.g., CD20) in fusion with a GFP reporter gene (reporter). Such a construct is also named reporter construct herein and described in detail above. These (reporter) constructs may be used on K562 cells, which do not express any endogenous levels of CD19 or CD20, to use them as target cells in the co-culture system of the present invention, as described in detail elsewhere herein.

Another big limitation of CAR T cell immunotherapies is that the uptake of CD19 by T cells through trogocytosis makes them a target for killing by other T cells, a phenomenon referred to as T cell fratricide⁵. The present inventors found that prolonged in vitro culture leads to the upregulation of the death receptor Fas, which renders CAR T cells susceptible to apoptosis and fratricide. As the result, in the described system roughly two thirds of T cells may die within the first 24 hours of co-culture (FIG. 8). The remaining one third of cells expand, but acquire a T cell exhaustion phenotype characterized by the expression of PD1, LAG3 and TIM3. T cell exhaustion has been associated with a poor clinical performance of CAR T cells. Thus, the described co-culture system addresses key clinical limitations of CAR T cell therapies, which can be addressed in sorting- or proliferation-based screens. For example, it is envisaged to FACS-purify CAR T cells that are GFP+ (have internalized the CAR for efficient signaling), but do not present CD19 on their surface. Alternatively, it is envisaged to screen for genes with a role in T cell trogocytosis, fratricide, exhaustion and proliferation through the experimental design outlined in FIG. 10, where CAR T cells are stimulated by either the TCR or CAR, and the gRNA dynamics over time are later compared. Thus, it is envisaged herein that in the methods of the present invention the engineered (immune) cells are screened for certain markers. Accordingly, it is envisaged herein that the (CAR) T cells are screened for certain markers. Accordingly, in the herein described methods the one or more markers(s) may be detected and the detection of one or more marker(s) associated with the (CAR) T cell are indicative of a (CAR) T cell property. The (CAR) T cell property may be associated with the desired phenotype. The term “(CAR) T cell property” may even be used synonymously with “phenotype”.

Accordingly, the invention relates to a method for functional screening of CAR T cells, the method comprising the steps of:
(a) isolating T cells from a sample, preferably a (peripheral) blood sample;
(b) transducing the T cells with a vector, the vector encoding a CAR and a gene editing molecule, preferably CRISPR guide RNA (gRNA);
(c) adding gene editing modifiers, preferably CRISPR modifiers to induce genetic modulation (e.g. CRISPR editing);
(d) selecting a CAR T cell population based on a desired (CAR) T cell property;
“Marker(s) associated with the (CAR) T” is used herein in the broadest sense and refers to basically all molecules or biological phenotypes that can be detected or measured qualitatively and/or qualitatively. The marker may be intracellular or on the surface of the cell. The marker may be endogenous or heterologous.

Accordingly, the invention relates to a method for functional screening of CAR T cells, the method comprising the steps of:
(a) isolating T cells from a sample, preferably a (peripheral) blood sample;
(b) transducing the T cells with a vector, the vector encoding a CAR and a gene editing molecule, preferably CRISPR guide RNA (gRNA);
(c) adding gene editing modifiers, preferably CRISPR modifiers to induce genetic modulation (e.g. CRISPR editing);
(d) selecting a CAR T cell population based on desired a desired (CAR) T cell property;
wherein detection of one or more marker(s) associated with the CAR T cell are indicative of said (CAR) T cell property.

Accordingly, the invention relates to a method for functional screening of CAR T cells, the method comprising the steps of:
(a) isolating T cells from a sample, preferably a (peripheral) blood sample;
(b) transducing the T cells with a vector, the vector encoding a CAR and a gene editing molecule, preferably CRISPR guide RNA (gRNA);
(c) adding gene editing modifiers, preferably CRISPR modifiers to induce genetic modulation (e.g. CRISPR editing);
(d) selecting a CAR T cell population based on a desired (CAR) T cell property;
wherein the CAR T cells are co-cultured with a second cell population, wherein the second cell population is engineered to express one or more reporter construct(s) and wherein detection of one or more marker(s) associated with the (CAR) T cell are indicative of a (CAR) T cell property.

In a preferred embodiment, the invention also relates to a method for functional screening of CAR T cells, the method comprising the steps of:
(a) isolating T cells from a sample, preferably a (peripheral) blood sample;
(b) transducing the T cells with a vector, the vector encoding a CAR specific for CD19 and a gene editing molecule, preferably CRISPR guide RNA (gRNA);
(c) adding gene editing modifiers, preferably CRISPR modifiers to induce genetic modulation (e.g. CRISPR editing);
(d) selecting a CAR T cell population based on a desired (CAR) T cell property;
wherein the CAR T cells are co-cultured with a second cell population, wherein the second cell population is engineered to express one or more reporter(s), wherein the reporter comprises and antigen and a reporter gene, wherein the antigen is CD19, the reporter gene is GFP and wherein detection of one or more marker(s) associated with the (CAR) T cell are indicative of a (CAR) T cell property.

It is evident that in the described methods both one or more reporter construct(s) and one or more marker constructs may be detected. However, it is also envisaged that e.g. the reporter construct(s) may only stimulate the (CAR) T cell and only the one or more marker(s) are detected (e.g. to screen for a certain property of the (CAR) T cell). Accordingly, the invention also relates to a method for functional screening of CAR T cells, the method comprising the steps of:
(a) isolating T cells from a sample, preferably a (peripheral) blood sample;
(b) transducing the T cells with a vector, the vector encoding a CAR and a gene editing molecule, preferably CRISPR guide RNA (gRNA);
(c) adding gene editing modifiers, preferably CRISPR modifiers to induce genetic modulation (e.g. CRISPR editing);
(d) selecting a CAR T cell population based on detection of the reporter construct on the surface or inside the CAR T cell and a desired (CAR) T cell property;

The invention also relates to a method for functional screening of CAR T cells, the method comprising the steps of:
(a) isolating T cells from a sample, preferably a (peripheral) blood sample;
(b) transducing the T cells with a vector, the vector encoding a CAR specific for CD19 and a gene editing molecule, preferably CRISPR guide RNA (gRNA);
(c) adding gene editing modifiers, preferably CRISPR modifiers to induce genetic modulation (e.g. CRISPR editing);
(d) selecting a CAR T cell population based on detection of the reporter construct on the surface or inside the CAR T cell and a desired (CAR) T cell property;
wherein the CAR T cells are co-cultured with a second cell population, wherein the second cell population is engineered to express one or more reporter(s), wherein the reporter comprises and antigen and a reporter gene, wherein the antigen is CD19, the reporter gene is GFP and wherein detection of one or more marker(s) associated with the (CAR) T cell are indicative of a (CAR) T cell property.

The marker associated with a (CAR) T cell (and detected and in context of the herein described methods) may be cell proliferation. Cell proliferation may be detected by the dropout or enrichment of gRNAs overtime, by fluorescent intracellular labeling (e.g., with CFSE), by marker expression (e.g., Ki-67), or by DNA dyes (e.g. DAPI).

The marker associated with associated with a (CAR) T cell (and detected and in context of the herein described methods) may be dropout or enrichment of gRNAs overtime.

The marker associated with associated with a (CAR) T cell (and detected and in context of the herein described methods) may be DNA content.

The marker associated with associated with a (CAR) T cell (and detected and in context of the herein described methods) may be a surface protein.

It is envisaged that in the herein described methods the cell type of the T cell is determined.

Accordingly, the marker(s) may be indicative of the cell type of the T cell. Markers that are indicative of the cell type of the T cell (and detected in context of the herein described methods) may be surface proteins such as CD3, CD4 and/or CD8. The skilled person is well aware of additional corresponding markers.

The marker(s) may be indicative of the engagement of the T cell with target cells.

It is envisaged that in the herein described methods T cell activation is determined. Accordingly, the marker(s) may be indicative of T cell activation. A marker that are indicative of T cell activation (and detected in context of the herein described methods) may be surface proteins such as CD69.

It is further envisaged that in the herein described methods T cell degranulation is determined. Accordingly, the marker(s) may be indicative of degranulation. A marker that is indicative of T cell degranulation (and detected and in context of the herein described methods) may be CD107a.

It is further envisaged that in the herein described methods cytokine production of the T cell is determined.

Accordingly, the marker(s) may be indicative of degranulation of cytokine production. Markers that are indicative of cytokine production (and detected in context of the herein described methods) may be IFNγ and/or IL2.

It is further envisaged that in the herein described methods expression of death receptors of the T cell is determined.

Accordingly, the marker(s) may be indicative of apoptosis. A marker that is indicative of apoptosis (and detected in context of the herein described methods) may be a death receptor, preferably Fas.

It is further envisaged that in the herein described methods T cell exhaustion is determined. Accordingly, the marker(s) may be indicative of T cell exhaustion. Markers that are indicative of T cell exhaustion (and detected in context of the herein described methods) may be LAG3 and/or TIM3.

Although completely evident it is pointed out that the skilled person is well aware of additional markers associated with T cells that are indicative for the above described T cell properties/phenotypes and T cell properties/phenotypes not explicitly mentioned herein.

To date, only a small number of genetic screens in primary human T cells have been described, and there is a single, low-quality example of a screen done directly on CAR T cells.

All of these screens employed positive FACS-based selection to enrich for populations of interest. This type of positive-selection screen can compensate for low genome engineering efficiencies by selecting phenotypes not typically found in the wild type population. For example, experimenters can sort very high levels of a marker, assuming that the upregulation is due to an effective genetic perturbation and unedited cells are excluded. CAMEL has important advantages over state-of-the-art methods. First, it is less damaging and maintains cells in a highly proliferative state. Second, CAMEL yield a much greater number of T cells for screening, so that researchers can achieve a higher library complexity, perform more readouts or both. This is evident from the present invention, where routinely 8 genome-wide readouts are performed from one T cell sample while maintaining a library complexity of >2000× per readout. An example of a successful sorting-based screen is shown in FIG. 9. Such a screen may be performed as follows. CAMEL may be used to establish a genome-wide pool of knockout CAR T cells, and an initial unsorted sample may be taken. Then the modified CAR T cells may be combined with target cells expressing the antigen CD19. This leads to target recognition, and the upregulation of the early activation marker CD69 in functional CAR T cells. At four hours, cells may be stained for CD69 (among other markers), fixed and separated by FACS into CD69−− (lowest 7%) and CD69++ (top 3.5%). Based on log 2 fold changes between the CD69−− and unsorted populations, gRNAs targeting CD69 can be identified as the top hits (FIG. 9a), highlighting the efficiency and specificity of the system. Next, CD69++ and unsorted populations can be compared, which is particularly relevant for optimizing immunotherapies. Genes can be identified that result in unusually high levels of CD69, indicating that they have a particularly high sensitivity or enhanced response to target cell stimulation (FIG. 9b).

To give an example, ACSL5 was found in a screen of the present invention. The role of this gene in primary human T cells has so far not been described. Based on the literature, ACSL5 expression is higher in systemic lupus erythematosus patients compared to healthy controls⁶. In the Jurkat T cell line, ACSL5 expression was increased during apoptosis induced by PMA-ionomycin, a very strong stimulus for TCR activation and siRNA downregulation of ACSL5 decreased the induced apoptosis⁷. The data suggest a role for ACSL5 in CAR T cell apoptosis, and that knockout in ACSL5 in CAR T cells could potentially make them more resistant to overactivation-induced apoptosis.

Proliferation-based readouts, where gRNAs encoding certain genetic perturbations drop out over time while others are enriched are hard to perform in a quantitative manner. Since the goal is to identify gRNAs that are depleted from a population, they suffer particularly from low editing efficiencies, low cell proliferation rates and noise associated with low library complexity. The present invention represents the first ever proliferation-based screen in primary human T cells. In FIG. 10-FIG. 13, key data from a successful genome-wide dropout screen are summarized. Such an experiment may be performed according to the experimental design in FIG. 10. Proliferation-based readouts are particularly important to identify and understand mechanisms that are essential for CAR T cells. Such mechanisms can then be enhanced, e.g. by overexpression of ORFs to improve CAR T cells. First T cells may be transduced with a lentiviral vector encoding the CAR and a genome-wide gRNA library and a Day 0 sample may be taken. Then cells may be electroporated with Cas9 mRNA to induce genome editing and may be selected with antibiotics to enrich for modified cells. The sample may then be split into two parts, the first may be stimulated via the TCR with anti-CD3/CD28 stimulation beads, and the second may be stimulated via the CAR with K562-CD19-mGFP target cells (as described above). The cultures may be sampled at Day 14 post electroporation.

Over time, depletion of essential genes can be observed compared to non-essential olfactory receptors widely used as a control (FIG. 11a). Using stringent cutoffs, statistically significant essential genes can be selected and Gene Ontology (GO) enrichment analysis can be performed (FIG. 11b). In the present invention the top 100 enriched GO terms are clustered based on the GO term hierarchy. This way, major essential processes were identified such as the cell cycle, transcription and translation, splicing, and ribosome biogenesis, but also a cluster of T cell specific processes (FIG. 11c). The latter includes IL2 signaling, and T cell activation, differentiation, and apoptosis.

Next, log 2 fold changes were calculated for selected genes and mapped onto a schematic of the T cell receptor pathway (FIG. 12). Both bead and target cell stimulated samples strictly required signaling through the IL2 receptor for their proliferation, in particular the genes IL2RG and JAK3. IL2 is the main cytokine required for T cell proliferation, and therefore used in all culture protocols. In addition, the co-receptor CD4 was essential for signaling via the TCR and CAR. The system of the invention also captured more subtle aspects of TCR signaling, such as co-regulation. For example, the positive co-receptor CD28 was depleted at Day 14, while its negative counterpart CTLA4 was enriched. Also interesting differences between TCR and CAR stimulated samples were found. As expected, the TCR subunits CD3E and CD3D were essential only in TCR-stimulated samples, where they are activated by the bead-bound anti-CD3 antibody. The inventors further surprisingly and unexpectedly found that knockouts of the kinase ZAP70 and the adapter protein LAT did not abolish signaling in CAR T cells, but rather resulted in increased proliferative potential. A knockout of ZAP70 or LAT could potentially reduce overactivation of CAR T cells, which can result in exhaustion and cell death.

With the herein described methods new genes can be discovered that could improve (CAR) T cell therapies. The screening data may be processed with the MAGeCK MLE algorithm, which yields beta values that are more representative of the effect size of genetic perturbations. The beta values may be normalized based on core essential genes, and compared between TCR and CAR stimulated samples (FIG. 13). This way knockouts that specifically promote CAR T cell proliferation (e.g. as in Tables 3 and 5), and knockouts that more generally promote both T and CAR cell expansion (Table 4) can be selected. Both gene sets are of great interest to improve T cell-based therapies. As described herein, knockout of RHOG is particularly advantageous for enhancing the pharmaceutical, e.g., anti-cancer, properties of T cells such as CAR T cells.

Thus, the invention also relates to the (immune) cells identified in the herein described methods. Accordingly, the invention relates to a T cell population comprising a lentiviral vector, wherein the lentiviral vector encodes a CAR and a CRISPR guide RNA (gRNA). The invention relates also to a T cell population comprising a lentiviral vector, wherein the lentiviral vector encodes a CAR and a CRISPR guide RNA (gRNA) and wherein the T cell is a CAR T cell.

It is also envisaged that the genes identified in the methods of the present invention are used to improve the therapeutic potential of CAR T cells. For example when a gene is identified to be essential for (CAR) T cell stimulation said gene may be overexpressed in CAR T cells to improve CAR T cell function. Genes that were found to be essential for stimulation of TCR or CAR are found in Table 1 and 2, respectively. Accordingly, the present invention further relates to CAR T cells in which these genes are overexpressed.

The present invention also identified genes where the corresponding knockout improves e.g. (CAR) T cell proliferation, e.g. RHOG. Accordingly, said genes (e.g. RHOG) may be deleted in (CAR) T cells to e.g. improve (CAR) T cell proliferation. Genes that were found to specifically promote CAR T cell proliferation when deleted are shown in Table 3. Gene knockouts that promote CAR T cell proliferation particularly strongly (e.g. RHOG KO) are shown in Table 5. Genes that were found to promote CAR T cell and T cell proliferation when deleted are shown in Table 4. Accordingly, the invention also relates to CAR T cells in which these genes are suppressed or deleted.

In particular, the invention relates to CAR T cells in which the expression of HAVCR2, VAMP4, SLC351B3, TRAF1, RHOG, NOTCH2, PHF8, DUSP6, DAW1, ZNF16, TCF4, PRSS3, LACTB, TMTC3, CD3001L1, MORC3 and/or CDKN2A is suppressed. Preferably herein, the expression of RHOG is suppressed or deleted in CAR T cells. Surprisingly, the knockout of RHOG more effectively enhanced the anti-tumor/anti-cancer effects of CAR T cells in vivo, e.g., in the NALM6 leukemia xenograft model described herein, as compared to other gene knockouts which also promoted CAR T cell proliferation in vitro, e.g. ZNF660, ZNF354, PRDM1, HAVCR2, ZAP70, LAT, VAMP4, DUSP6, NOTCH2, TRAF1, CDKN2A, MORC3, PHF8, or LACTB. It needs to be further stressed that, unexpectedly, this enhancement was also seen over the knockout of PRDM1, which had been previously shown to improve CAR T cell therapy (Dai, Nat Biotechnol. 2023 September; 41(9):1239-125; Yoshikawa (2022), Blood 139 (14): 2156-72), and which therefore served as positive control; see, e.g., Example 2 and FIG. 16.

TABLE 1
Essential genes identified for TCR stimulation

| | | | | | |
|---|---|---|---|---|---|
| CD69 | PDCD1 | PSMB4 | MYC | F8A1 | CD4 |
| DNAJC9 | POLR2L | IMP3 | FAU | RPL8 | RPL13 |
| EXOSC6 | IFNG | NOP14 | RPS8 | RPL6 | SLC7A6OS |
| RPLP2 | FAM231A | RPL36 | PPP1R10 | RPS26 | SRSF1 |
| USP17L19 | HSPA5 | EIF2S3 | ATP6AP1 | C19orf52 | SLC1A5 |
| ZNHIT2 | PLK1 | NOM1 | RPS9 | IL2RG | FDX1L |
| PRELID1 | RPL27 | UXT | CCR7 | GFER | LSM2 |
| F8A3 | PSMB3 | DUT | SELL | THOC3 | RPL17-C18orf32 |
| SPATA5L1 | WASH1 | GADD45GIP1 | USP17L30 | MRPL57 | MRPS12 |
| EIF2S2 | YARS2 | JAK3 | PTPMT1 | COX17 | NSL1 |
| EIF6 | MRPL41 | WDR43 | TUBB | IRF4 | MRPL17 |
| RPS16 | IL2 | NOP16 | PSMA7 | RPL19 | GNL3 |
| LOC100507462 | ATP6V0C | CDC37 | NUS1 | RPS6 | RPS10 |
| ATP6V1A | SNRPE | POLD2 | ZCRB1 | RPS29 | MAT2A |
| DAD1 | RPS19 | MARS2 | DHX33 | VHL | KRTAP20-3 |
| BUB3 | RPS27A | PRPF8 | RPS13 | SOD2 | RPL31 |
| TERF2 | LOC101060389 | GPN2 | JUNB | USP17L28 | ADSL |
| RPL37 | MCM6 | DDX28 | RPS20 | TBC1D3C | RPS23 |
| CCT4 | CITED2 | CYCS | SETDB2 | ALG2 | PES1 |
| PRMT1 | ARL2 | HSPD1 | RPE | POLR3B | SRSF2 |
| VMP1 | HIST2H3C | EIF3C | PABPC1 | AURKB | TTC27 |
| NUTF2 | RPL3 | SLC25A28 | CCT6A | CYC1 | RNF113A |
| MALT1 | SF3B5 | SPDYE2 | PPP4C | HIST1H2BN | DDX54 |
| GPKOW | SMU1 | RPL11 | AKIRIN2 | DYNC1I2 | PTPRC |
| WEE1 | RPL34 | EMG1 | NBPF9 | THOC5 | VCP |
| HSPA8 | SARS2 | TXN | MRPS34 | GINS2 | RPL5 |
| TP53RK | CDK1 | RANGAP1 | RPL23 | HIST2H2BF | RPL23A |
| PRIM1 | RAD9A | HMGCR | NOP2 | NBPF15 | WDR77 |
| LARS | SF3A2 | CDK6 | KRT18 | STARD4 | TIMM10 |
| NAA10 | SGCB | RPL35 | ZNHIT3 | XRCC5 | USP39 |
| CRNKL1 | NKAP | UBE2D3 | MYBBP1A | RPL18 | OTUD4 |
| PSMA6 | | | | | |

TABLE 2
Essential genes identified for CAR stimulation

| | | | | | |
|---|---|---|---|---|---|
| PSMB4 | CD4 | PDCD1 | DNAJC9 | FAU | RPL13 |
| RPL8 | RANGAP1 | EXOSC6 | HIST1H2AI | RPS8 | HSPA5 |
| RPL6 | IMP3 | IL2RG | RPL27 | MEPCE | MYC |
| IFNG | SNRPD3 | RPL36 | RPS19 | SRSF1 | FAS |
| SPDYE6 | POLR2L | ANAPC11 | POLR3E | PRELID1 | TICRR |
| NOP14 | RPL11 | ZNHIT2 | RPS13 | NOP58 | RPL19 |
| IRF4 | USP17L29 | GINS2 | GAPDH | LSM2 | HSPD1 |
| RPL17-C18orf32 | RPL37 | PLCB4 | PRPF38A | TERF2 | TUBB |
| ATP6AP1 | PSMA5 | VARS | EIF6 | PRMT1 | SF3B5 |
| JAK3 | USP17L28 | HNRNPC | DUT | ARMC7 | SELL |
| ATP6V0C | RPS11 | RPS16 | DNTTIP2 | IL2 | USP17L19 |
| NOP16 | THOC3 | RPS10 | NLE1 | EMG1 | PSMA6 |
| RPL10A | ZNF675 | CCNH | PRPF8 | RPL23A | GFER |
| CDK6 | UBE2D3 | CLNS1A | PHB2 | NOP2 | SPATA5L1 |
| LOC100507462 | LRRC37A | RPS4X | RPS20 | ALG2 | SLC25A28 |
| AURKB | SPDYE2 | USP39 | VPS28 | HSPA9 | NPIPB6 |
| SACM1L | TRMT112 | PSMA7 | RPL3 | PDRG1 | |

TABLE 3
Genes that specifically promote CAR T cell proliferation when deleted

| | | | | | |
|---|---|---|---|---|---|
| HAVCR2 | SLC35B3 | CASP7 | ZAP70 | LIPA | MAGEB6 |
| PCNXL4 | PRG4 | NRM | ACP2 | LHFPL4 | PLA2G16 |
| SLC19A3 | MORC3 | STAC3 | GLIS3 | TRAF1 | RHOG |
| CNTROB | MUC16 | SRD5A2 | ELAC1 | C1orf216 | GOLGA1 |
| DOCK2 | PATL2 | SERPINB13 | ITIH2 | CCDC168 | NOTCH2 |
| GFRA1 | LAT | C9orf135 | DOCK4 | SLC13A3 | PRSS50 |
| TBX5 | GALNTL6 | CCDC169-SOHLH2 | TSPYL2 | BRIP1 | ERICH3 |
| GYG2 | SERINC1 | RAB11FIP3 | PKD2 | ADAM8 | FGF17 |
| PDCD5 | TFB1M | ERCC4 | C1orf21 | OR51F1 | UGT1A6 |
| CXorf67 | TXNDC9 | BLOC1S5 | GALNT10 | LIPI | HR |
| PTBP2 | F10 | GDPD4 | SEC23IP | PLAT | ZFP82 |
| LILRA4 | EYS | PRKG2 | MTAP | ALG12 | SYCP2 |
| SMIM21 | NUP35 | MCHR2 | TSPAN2 | PTPN21 | CADPS2 |
| EFCAB2 | AMMECR1 | CLDN23 | ATXN7L3 | SNX11 | PTHLH |
| PYHIN1 | SYNPR | SQRDL | NCF4 | CSTF2T | C5orf34 |
| RSPO1 | PIK3R3 | COL4A4 | ZCCHC18 | IRF8 | HMGXB4 |
| PCDH9 | EIF3K | LAPTM4A | TTC7B | GRM8 | NLK |
| SEL1L3 | TNFRSF9 | KLHDC7A | EFCAB6 | IL1A | POU5F1B |
| C11orf73 | ARHGAP30 | SLC23A1 | CLPS | SLC5A12 | MAPK8IP2 |
| KIAA1462 | AQP1 | IRX5 | LCTL | ASTE1 | SLC12A4 |
| ITGA2B | STYK1 | SPATA12 | AK7 | MICAL3 | SLC37A4 |
| EPS8L1 | CNR1 | IL20 | CNBD2 | CSF1 | ACSM1 |
| MEDAG | PARVB | ECI1 | ZER1 | MXRA5 | C7orf76 |
| TM4SF4 | SH3RF2 | HMSD | DNAH5 | CALY | EGLN3 |
| ELOVL3 | ANKRD1 | DMXL2 | ZNF354B | LY6H | FAM127B |
| HMGN5 | IRF2 | PLCG1 | PHC2 | SLC25A48 | CALR3 |
| C2CD5 | ALB | YWHAH | DENND4B | CCDC110 | PCDHA6 |
| KHK | C19orf54 | MAP2K3 | PLXDC2 | ACBD5 | RAPGEFL1 |
| NDE1 | PIP4K2A | LIN7A | OR8I2 | SYK | ZFP69B |
| RICTOR | FAM105A | GIT1 | PPP1R3D | FOXJ2 | GPR37 |
| TXN2 | CAMSAP2 | MYOT | ZHX2 | RAB3D | TKTL1 |
| ATF1 | HDAC4 | NPHP3 | OR8G2 | PRKCG | OR9A4 |
| NME3 | MKNK2 | FAM13B | TINAGL1 | RPGRIP1L | PKIB |
| CD80 | SERPINI2 | LOC100130370 | KLRG1 | SCCPDH | SPATA4 |
| ATP9A | | | | | |

TABLE 4
Genes that promote both T and CAR T cell proliferation when deleted

| | | | | | |
|---|---|---|---|---|---|
| ZNF354A | PRDM1 | ZNF660 | CTLA4 | VAMP4 | PRSS3 |
| LACTB | DAW1 | CASP3 | IL37 | CCL2 | ZNF16 |
| APEH | TCF4 | SAXO1 | SYN1 | C8G | KCNT2 |
| CDKN2A | TMTC3 | SYNGAP1 | SRC | DUSP6 | TP53 |
| MATN2 | EHMT2 | DMRTC1 | SIK3 | ZNF645 | CCDC92 |
| LRRC31 | CD300LB | QPCT | AMPH | HNF1B | SIAE |
| RHOBTB2 | SCHIP1 | SMARCD3 | MGC50722 | CLEC7A | SLAIN1 |
| FMOD | KLLN | FXYD1 | HSF4 | OTOGL | MLC1 |
| DNAH11 | C6orf47 | RANBP9 | PRAM1 | NFE4 | VWA2 |
| MMP13 | DYRK3 | MED13 | USP22 | PHGR1 | IVL |
| PBLD | LOC149373 | GPRC5D | LOC645382 | MMD | AMIGO1 |
| DOK7 | KISS1 | MFSD4 | MSLN | LRFN4 | STAC2 |
| PAM | PRICKLE2 | BTBD8 | GPR15 | PER1 | C20orf196 |
| GPC1 | LCE2A | SGSM1 | IL22RA2 | MAGEA5 | SLC40A1 |
| ZNF200 | TRAF3IP1 | SAMD12 | LGI2 | TTC3 | MTFR1 |
| SFXN3 | ATP13A5 | POC1B-GALNT4 | OR51M1 | MTURN | CLDN11 |
| CCDC17 | RNASE6 | CELF6 | ERGIC3 | SLC16A2 | MAPK1IP1L |
| LCE3B | TMEM108 | IQCE | FTSJ1 | SYT1 | CITED4 |
| TMEM213 | CXCR3 | ADCY3 | WDR6 | BGLAP | NRG4 |
| MS4A12 | WIZ | C16orf47 | PPP1R32 | LRFN3 | PAMR1 |
| CALML3 | RGAG1 | CCDC89 | RAB5B | APELA | HOGA1 |
| UNC13B | ZNF75D | SNAP47 | TMEM101 | ZNF776 | KANK4 |
| ASCL2 | P2RY2 | OR14I1 | PHLDB3 | POU2F2 | ZBED2 |
| BBC3 | MUC21 | PARVG | TACR3 | C2orf66 | PTCHD3 |
| MPP1 | OAF | ASCL4 | NDRG1 | TNFSF13 | TMEM125 |
| PNMAL1 | SMR3B | SGCE | TBC1D14 | GDF5 | HNF4G |
| NBL1 | COX8C | PLEKHO2 | C11orf44 | | |

CAR T cells for clinical or preclinical models are typically produced with excessive amounts of lentivirus to ensure a high transduction efficiency. This results in multiple CAR integrations per cell, higher CAR expression levels and a greater antigen sensitivity in vivo. Genetic screens, however, require a much lower lentivirus concentration to achieve a single integration and defined genetic perturbation in each cell. A mouse tumor model induced by NALM6 cell injection may be used to compare treatment effects by CAR T cells produced with the herein described optimized lentivirus concentration for single gRNA integrations, or a 30× higher lentivirus concentration. Despite a potential disadvantage for CAR expression, we observed efficient tumor clearance in both conditions (FIG. 14) was observed, showing that the described platform supports pooled in vivo screens.

TABLE 5
gRNA enrichment for gene knockouts improving CAR T cell
proliferation (log fold change between Day 14 post-stimulation
with activation beads or target cells vs Day 0).

| Gene | Dynabeads_log2FC | TargetCells_log2FC |
|---|---|---|
| ZNF354A | 1.5 | 2.0 |
| ZNF660 | 1.1 | 2.0 |
| CTLA4 | 1.0 | 2.0 |
| HAVCR2 | 0.2 | 1.8 |
| PRDM1 | 1.8 | 1.7 |
| SLC35B3 | 0.4 | 1.6 |
| ZAP70 | −0.3 | 1.4 |
| LACTB | 1.2 | 1.3 |
| CD300LB | 0.7 | 1.2 |
| TMTC3 | 0.8 | 1.2 |
| MORC3 | −0.1 | 1.2 |
| DAW1 | 1.1 | 1.2 |
| PRSS3 | 1.3 | 1.2 |
| TRAF1 | 0.4 | 1.1 |
| RHOG | 0.4 | 1.1 |
| VAMP4 | 1.5 | 1.1 |
| NOTCH2 | 0.4 | 1.0 |
| LAT | −0.2 | 1.0 |
| CDKN2A | 1.1 | 1.0 |
| TCF4 | 1.4 | 0.7 |
| ZNF16 | 1.4 | 0.7 |

As already mentioned above, the CAR T cells of the present invention can be used for pharmaceutical purposes. Accordingly, the invention relates to the use of the CAR T cells of the invention in the prevention, treatment or amelioration of a disease. The invention relates to the use of the CAR T cells of the invention in the prevention, treatment or amelioration of cancer. In other words, the invention relates to the use of said CAR T cells as a medicament. It is also envisaged that CAR T cells of the present invention form part of a composition. Said composition may comprise one or more of the CAR T cells of the present invention. Said composition may be a pharmaceutical composition optionally further comprising a pharmaceutically acceptable carrier and/or diluent. Said composition may be used for pharmaceutical or diagnostical purposes. Accordingly, the invention relates to the use of a pharmaceutical composition comprising the CAR T cell of the invention in the prevention, treatment or amelioration of a disease, preferably cancer. In other words the invention relates to the use of a pharmaceutical composition comprising the CAR T cell of the invention as a medicament.

The invention also relates to a method of treatment wherein the CAR T cell of the invention is administered to a patient in need thereof. The invention also relates to a method of treatment of cancer wherein the CAR T cell of the invention is administered to a patient in need thereof.

The invention further relates to the following items:
1. Method for functional screening of engineered immune cells, the method comprising the steps of:
   (a) isolating immune cells from a sample;
   (b) transducing the immune cells with a vector, the vector encoding a CAR and a gene editing molecule, preferably CRISPR guide RNA (gRNA);
   (c) adding gene editing modifiers, preferably CRISPR modifiers to induce genetic modulation (e.g. CRISPR editing);
   (d) selecting a immune cell population based on desired phenotype.
2. The method of item 1, wherein the immune cell is a T cell, preferably a CAR T cell or a CTL.
3. The method of item 1 or 2, wherein the sample is a blood sample, optionally obtained from a healthy donor or a patient, preferably cancer patient.
4. The method of any one of items 1 to 3, wherein the vector is a lentiviral vector or a Sleeping Beauty (SB) transposon, preferably wherein the lentiviral vector configuration enables single-cell sequencing.
5. The method of item 4, wherein the lentiviral vector comprises the gRNA at the 3′ long terminal repeat (LTR).
6. The method of any one of items 3 to 5, wherein the lentiviral vector comprises the CAR 5′ of the gRNA.
7. The method of any one of items 2 to 5, wherein the CAR is expressed under the EF1α or the CMV promoter.
8. The method of any one of items 2 to 6, wherein the CAR comprises a leader peptide, a single-chain variable fragment (scFv) antibody and optionally the hinge- and transmembrane domains of CD8, the intracellular co-stimulatory domains of CD28 and/or 41BB and/or the CD3ζ chain.
9. The method of item 8, wherein the scFv antibody is specific for CD19, BCMA, GD2, HER2, CD123, CD138, CD20, CD22, CD38 CDK5, IgK, LeY, NKG2D, ROR1, WT1, c-met, CAIX, CD70, CEA, EGFR, EpCAM, EphA2, FAP, GD2 HER2, IL13RA2, LeY, MAGEA3, MAGEA4, MART1, Mesothelin, MUC1, MUC16, NY-ESO-1, PD-L1, PSCA, PSMA, or VEGFR2, preferably CD19.
10. The method of any one of items 1 to 9, wherein the CRISPR modifiers comprise an endonuclease.
11. The method of item 10, wherein the endonuclease is SpCas9, preferably SpG or SpRY.
12. The method of any one of items 1 to 11, wherein the CRISPR modifiers are added in the form of RNA, in particular mRNA.
13. The method of item 12, wherein the CRISPR modifiers are introduced via electroporation.
14. The method of any one of items 2 to 13, wherein the T cells are co-cultured with a second cell population.
15. The method of item 14, wherein the second cell population is engineered to express one or more reporter construct(s).
16. The method of item 15, wherein the reporter construct comprises an antigen and a reporter.
17. The method of item 16, wherein the antigen is selected from the group consisting of BCMA, GD2, HER2, CD123, CD138, CD20, CD22, CD38, CDK5, IgK, LeY, NKG2D ROR1, WT1, c-met, CAIX, CD70, CEA, EGFR, EpCAM, EphA2, FAP, GD2, HER2, IL13RA2, LeY, MAGEA3, MAGEA4, MART1, Mesothelin, MUC1, MUC16, NY-ESO-1, PD-L1, PSCA, PSMA, and VEGFR2, preferably wherein the antigen is CD19 and preferably wherein the reporter is GFP.
18. The method of any one of items 14 to 17, wherein the detection of the reporter construct on the surface or inside the T cell is indicative of engagement of the T cell with the second cell population.
19. The method of any one of items 14 to 18, wherein detection of one or more marker(s) are indicative of a T cell property.
20. The method of item 19, wherein the marker is proliferation detected by the droupout or enrichment of gRNAs overtime, by fluorescent intracellular labeling (e.g. with CFSE), by marker expression (e.g. Ki-67), or by DNA dyes.
21. The method of item 19, wherein the marker(s) is/are indicative of the cell type of the T cell.
22. The method of item 21, wherein the marker(s) is/are CD3, CD4 and/or CD8.
23. The method of item 19, wherein the marker(s) is/are indicative of the engagement of the T cell with target cells.
24. The method of item 19, wherein the marker(s) is/are indicative of T cell activation.
25. The method of item 24, wherein the marker is CD69.
26. The method of item 19, wherein the marker(s) is/are indicative of degranulation.
27. The method of item 26, wherein the marker is CD107a.
28. The method of item 19, wherein the marker(s) is/are indicative of cytokine production.
29. The method of item 28, wherein the marker is IFNγ and/or IL2.
30. The method of item 19, wherein the marker(s) is/are indicative of apoptosis.
31. The method of item 30, wherein the marker is a death receptor, preferably Fas.
32. The method of item 19, wherein the marker(s) is/are indicative of T cell exhaustion.
33. The method of claim 32, wherein the marker is PD1, LAG3 and/or TIM3.
34. A T cell population comprising a lentiviral vector, the vector encoding a CAR and a CRISPR guide RNA (gRNA).
35. The T cell population of item 34, wherein the T cell is a CAR T cell.
36. A CAR T cell population in which expression of HAVCR2, VAMP4, SLC35B3, TRAF1, RHOG, NOTCH2, PHF8, DUSP6, DAW1, ZNF16, TCF4, PRSS3, LACTB, TMTC3, CD300LB, MORC3 and/or CDKN2A is suppressed.

The invention is illustrated by the appended non-limiting Figures and Examples.

### BRIEF DESCRIPTION OF THE DRAWINGS

FIG. 1—The CAMEL workflow (CRISPR applied by mRNA electroporation+lentivirus).

FIG. 2—CAR T cell tumor killing model. a, CAR expression in CAR T cells engineered with CROPseq-CAR constructs. b, Bioluminescence assay demonstrates successful tumor lysis by CAR T cells. c, CRISPR-engineered NALM6 cell lines with defined levels of CD19 surface expression.

FIG. 3—Laboratory pipeline for genetic screens in CAR T cells.

FIG. 4—Optimization of lentivirus transduction. a, Transduction rates after T cell stimulation with different regents. b, T cell viability for different transduction culture conditions. c, T cell transduction rates for different cell culture conditions. d, Lentivirus titer quantified by RT-qPCR. e, Lentivirus titration in CD4 and CD8 T cells. f, Number of lentivirus integrations in single cell-expanded clones quantified by gRNA sequencing. g, Comparison of lentivirus integration number detected by gRNA sequencing and by ddPCR.

FIG. 5—Optimization of T cell culture conditions. a, IL2 titration to identify the concentration for optimal T cell expansion. b, Expansion rate of CD4 and CD8 T cells cultured alone or together. c, CD4:CD8 ratio over time during CD3 T cell culture.

FIG. 6—Optimization of antibiotic selection. a, T cell viability data for a titration of puromycin on untransduced cells. b, CAR expression after 10 days of puromycin selection. c, T cell viability data for a titration of blasticidin on non-electroporated cells and cells electroporated with BSD mRNA. d, CD44 expression levels after knockout using CAMEL technology.

FIG. 7—Readouts of CAR T cell function. Left panel, CAR T cells are combined with an engineered target cell line, e.g. K562 cells expressing the antigen recognized by the CAR (CD19), fused to GFP. CAR T cells engage with their targets, and the TCR gets internalized for efficient signaling. In the process, the antigen CD19 in fusion with GFP is taken up by functional CAR T cells. Thus, CD19 and GFP can serve as markers of CAR T cell engagement. CAR T cells then initiate downstream signaling, upregulating the early activation marker CD69. This results in the production of cytokines such as IL2 and IFNγ. When CAR T cells cytotoxically kill their target cells, they expose the degranulation marker CD107a on their surface. Right panel, expression levels of the marker proteins at defined timepoints after target cell stimulation. Most markers show a high dynamic range, ideally suited for genetic screening.

FIG. 8—Readouts of CAR T cell exhaustion, apoptosis, and fratricide. The system employed by the inventors recapitulates key limitations of CAR T cell therapies such as exhaustion due to tonic signaling and T cell fratricide. a-b, when co-cultured with target cells expressing the antigen recognized by the CAR (e.g., CD19), CAR T cells acquire the antigen via trogocytosis. c, Long-term ex vivo culture leads to the expression of the death receptor Fas on CAR T cells. The marker Fas has ideal properties for genetic screening. d, Roughly two thirds of CAR T cells die within the first 24 hours, when co-cultured with CD19+ target cells. CD20-expressing target cells are not recognized by the CAR and did not lead to T cell death. e, Expression of characteristic exhaustion markers PD1, LAG3 and TIM3 at defined timepoints after target cell stimulation. The exhaustion markers are suitable for negative dropout screens when sorting the positive population.

FIG. 9—Genome-wide screen in CAR T cells with a CD69 sorting readout. a, gRNAs targeting CD69 are strongly enriched in the CD69−− population (lowest 7% of CD69 expression), validating our approach. b, The inventor's screen identifies gene knockouts with the potential to improve CAR T cell sensitivity and activation.

FIG. 10—Experimental design to identify genes that improve CAR T cell fratricide and exhaustion. After an initial pre-expansion period of 2 weeks, T cells are transduced with a lentiviral construct to deliver the CAR and a CRISPR gRNA library. Two days later, cells are electroporated with Cas9 mRNA and mRNA for blasticidin resistance. Antibiotic selection with puromycin selects for the lentiviral construct, and blasticidin selects for successful electroporation. Next, the sample is split into two parts, which are stimulated either with anti-CD3/CD28 beads to activate the TCR, or with K562-CD19-mGFP target cells to activate the CAR. Samples are taken before electroporation (Day 0), and at Day 14 post electroporation.

FIG. 11—Genome-wide screen in CAR T cells with a proliferation-based readout. The results are based on 4 individual donors, and two independent screening experiments. This type of screen is notoriously challenging and to the inventor's knowledge has never been described for primary human T cells. a, Dropout of essential genes versus non-essential genes between Day 14 and Day 0, in samples stimulated via the TCR (left) or CAR (right). b, Identification of essential genes in both datasets. We plot the log 2 fold change between Day 14 and Day 0 against the adjusted p-value as calculated by MAGeCK RRA. We apply stringent cutoffs adjusted p-value<0.01 and a log 2 fold change <−1. Lists of the highlighted essential genes are shown in Tables 1 and 2. c, Gene Ontology enrichment analysis and clustering for the identified essential genes. As expected, most clusters correspond to essential cellular processes. In addition, there is one T cell specific cluster, which includes IL2 signaling, T cell activation, differentiation, and apoptosis.

FIG. 12—CAR T cells stimulated via the TCR (with anti-CD3/CD28 beads) or CAR (with K562-CD19-mGFP target cells) were sampled at Day 14, and compared separately to the Day0 condition. Log 2 fold changes summarized in the heatmap were mapped onto a schematic of the T cell receptor pathway, with the bead-treated sample on top, and the target cell-treated sample at the bottom.

FIG. 13—Identification of gene knockouts that improve (CAR) T cell fitness.

A) Analysis of the dataset with the MAGeCK MLE algorithm yields beta values that can be normalized to essential genes and compared between conditions. The normalized beta values are indicative of the effect size of gene knockouts, which allows to compare between conditions. This way, genes that specifically promote CAR T cell proliferation (Table 3), or more generally both T and CAR T cell proliferation (Table 4), when deleted, can be selected. The gene knockouts identified here could enhance CAR T cell therapies. Lists of the highlighted genes are shown in Table 5. It is envisioned to target these genes, for example RHOG, to enhance T cell therapies.

B) MAGeCK MLE beta values for RHOG.

FIG. 14—The inventor's workflow supports pooled in vivo screens. This experiment is based on a mouse systemic tumor model induced by NALM6 injection into the teil vein. NALM6 cells were modified to express firefly luciferase, and can thus be monitored by bioluminescent imaging. The inventors observed complete tumor clearance when injecting CAR T cells 4 days after tumor induction. Importantly there was no reduction in tumor growth suppression when using a low lentivirus concentration compatible with single gRNA integrations and genetic screening. To the inventor's knowledge this is the first example of an in vivo model run at such a low lentivirus concentration.

FIG. 15—Schematic map of vector PD0039 (SEQ ID NO:1).

FIG. 16. RHOG knockout enhances in vivo tumor control by CAR T cells in a leukemia xenograft model.

(A) Experimental timeline. Anti-CD19 CAR T cells containing knockouts of either RHOG or PRDM1 were engineered. As negative control, the inventors also edited at the PPP1R12C safe harbor locus (intron1), referred to as SafeHarbor. CD3 T cells were isolated from whole blood and stimulated with TCR activation reagent. T cells were transduced at day 2 with CROP-seq-CAR lentivirus containing 8 gRNAs targeting the gene of interest (4 from the previously used genome-wide library, and 4 independently designed; see Table 8). Puromycin selection was started on day 5 (and maintained throughout the CAR T cell production). Cells were restimulated on day 7, and electroporated 3 days later with Cas9 and BSD mRNA to knock out RHOG (or PRDM1). Blasticidin was added 24 hours later and washed out after 1 day. The NSG mouse model was timed such that freshly produced CAR T cells could be administered at day 6 post-electroporation. NSG mice were first injected with 0.5 million NALM-6 cells to model a systemic CD19+ B cell leukemia, and 5 days later were treated with 0.6 or 1.2 million CAR T cells.

(B) Location of 8 gRNAs used for CAR T cell editing relative to the coding sequence (CDS) of RHOG or PRDM1.

(C) The gRNA representation in CAR T cell samples prior to injection was assessed by gRNA sequencing.

(D) Luciferase assay to measure tumor lysis after 18 hours of co-culture with CAR T cells.

(E) Representative live imaging (IVIS) for each treatment group (n=1 donor).

(F) Tumor growth measured by luciferase-based bioluminescence live imaging for a representative CAR T cell donor (0.6 M CAR T cell dose).

(G) Quantified fold reduction in tumor load between RHOG KO CAR T cells, PRDM1 KO CAR T cells and control SafeHarbor edited CAR T cells (0.6 M CAR T cell dose).

(H-I) Survival curves for the cohort in (D); pooled data for n=6 independent T cell donors.

(J) Tumor growth was measured by luciferase-based bioluminescence live imaging for a representative CAR T cell donor (1.2M CAR T cell dose).

(K) Survival curves for the cohort in (G); pooled data for n=3 independent T cell donors.

FIG. 17. RHOG knockout enhances in vivo tumor control by CAR T cells in a solid tumor xenograft model.

(A) Experimental timeline. Anti-CD19 CAR T cells with either RHOG knockout or SafeHarbor edit were engineered with Cas9 RNPs with one selected gRNA per target (SEQ ID NO: 25 for the RHOG knockout). CD3 T cells were isolated from whole blood and stimulated with TCR activation reagent. On day 2-3, T cells were electroporated with the RNPs. Immediately after electroporation, to deliver the CAR, a CROP-seq-CAR lentivirus containing a non-targeting gRNA was added to the medium for transduction, and puromycin selection was started 2 days later. CAR T cells were restimulated on day7 and cultured for one more week prior to injection into mice. NSG mice were subcutaneously injected with 1 million CD19+K562 cells and, 5 days later treated with 1.2 million CAR T cells injected intravenously.

(B) Tumor growth measured by caliper.

(C) Tumor size at day 12.

(D) Survival curves for the cohort in (B); data for n=1 independent T cell donor.

FIG. 18. RHOG knockout CAR T cells possess increased proliferative and metabolic potential on a molecular level.

(A) Experiment design to profile the RHOG and SafeHarbor knockout CAR T cells in vitro on the molecular level. CAR T cells were stimulated either via CAR in a co-culture with target cells or via TCR by restimulation. In vitro assays were performed within one day before or after the in vivo injection.

(B) Validation of increased RHOG expansion in single knockout CAR T cells. On the mouse injection day, CAR T cells were stimulated via TCR

(C) Network analysis of upregulated GO Biological Processes in CD4+ and CD8+ CAR T cells.

(D) Results of the gene set enrichment analysis for GO Biological Processes identified in selected clusters from (C).

(E) Differences in gene expression in RHOG vs SafeHarbor knockout CAR T cells for S phase genes.

(F) T cell differentiation status was profiled over 10 days of co-culture of CAR T cells and tumor cells. Represented is the difference of the fraction of central memory (CD45RO+CD62L+) cells in RHOG vs SafeHarbor knockout CAR T cells.

(G) Summary of RHOG knockout effects on CAR T cells on a molecular level that can lead to enhanced therapeutic effect of RHOG inactivation in T cell therapies.

FIG. 19. In vitro characterization of RHOG knockout CAR T cells.

(A) RHOG genome editing was confirmed by locus sequencing. “Total editing” denotes % Frameshift+% in-frame mutations.

(B) CAR expression level measured by flow cytometry in CAR T cells one day pre-injection.

(C) CD4 and CD8 composition of CAR T cells one day pre-injection.

(D) Principal component analysis (PCA) of normalized RNA-seq data showing samples by cell type, T cell donor, time point, and knockout.

(E) Number of differentially expressed genes (log FC>0.5, adj. p-val<0.05).

(F) Number of differential GO Biological Processes (FDR<0.05).

(G-H) Network analysis of upregulated GO Biological Processes in CD4 (G) and CD8 (H) CAR T cells.

(I) T cell differentiation status was profiled over 10 days of co-culture of CAR T cells and tumor cells. Representative flow cytometry data.

(J) —blank—

(K) Fraction of central memory (CD45RO+CD62L+) CAR T cells over time.

(L) CAR T cell functional markers assessed by flow cytometry after 16 hours of co-culture with K562-CD19 cells.

(M) CAR T cell exhaustion markers assessed by flow cytometry after 3 days of co-culture with K562-CD19 cells.

FIG. 20. Base editing tiling screen in CAR T cells identifies therapeutically relevant amino acid targets in RHOG.

(A) Experiment design. Fitness screen was performed in CD4 CAR T cells with CAR restimulation, and cells were sampled at day 0 and 14 of restimulation for comparison of gRNA distributions. Five CRISPR editors were electroporated into transduced CAR T cells. gRNA library was designed for tiling RHOG with all possible gRNAs.

(B) Number of gRNAs introducing specific mutation types in RHOG for each CRISPR editor.

(C) Functional variant map for RHOG. z-scores from base editing fitness screens are plotted for each gRNA across the gene body with known functional domains and key amino acid positions. Top enriched gRNAs are labeled with predicted edited amino acid mutations. Screen z-scores are calculated independently for each base editor and plotted together for comparison.

(D) Amino acids that are most strongly impacted by gRNA targeting (C) are highlighted on the corresponding protein structure representation. Hydrogen bonding was predicted by pyMol.

### EXAMPLES

The Examples illustrate the invention.

### Example 1. Functional Genetic Screening in Human Chimeric Antigen Receptor (CAR) T Cells—High-Level Overview of the CAMEL Technology

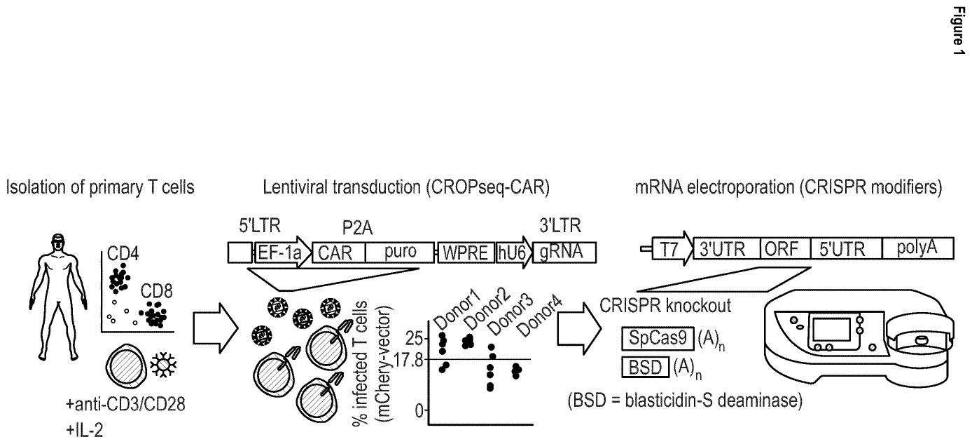

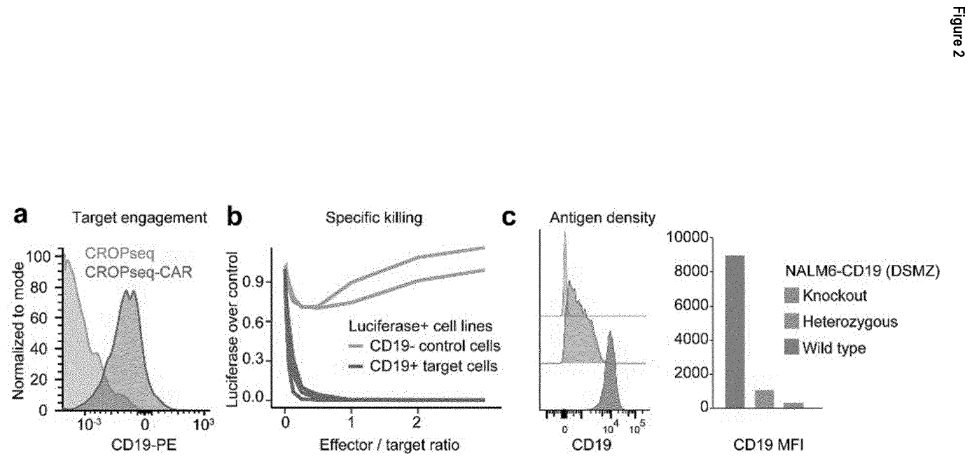

The invention describes, inter alia, a laboratory pipeline for functional genetic screens in human chimeric antigen receptor (CAR) T cells. It is based on and applies the inventive CAMEL technology (FIG. 1a) for effective CRISPR editing to primary human T cells, to optimize them for immunotherapy applications.

First, T cells are isolated from human peripheral blood (typically blood donations from healthy volunteers but potentially also blood from cancer patients), then cultured and stimulated under defined conditions. While undergoing an optimized culture and stimulation regimen, they are first transduced with a lentiviral vector to express a chimeric antigen receptor (CAR) and CRISPR guide RNA (gRNA) cassette. Later, CRISPR modifiers are delivered as in vitro transcribed messenger RNA (mRNA), resulting in efficient CRISPR editing. Selection with antibiotics or by fluorescence-activated cell sorting (FACS) is used to enrich a pool of modified T cells.

To test the performance of T cells, the inventors developed a co-culture system with engineered target cells. Using this system, CAR T cells can be effectively isolated based on their cell type (CD4, CD8), engagement with target cells (CD19, GFP), T cell activation (CD69), degranulation (CD107a), cytokine production (IFNg), the expression of death receptors (Fas), T cell fratricide (culture overtime, Fas, ratio between CD19 and GFP), or T cell exhaustion (PD1). Importantly, the inventors have combined these markers into novel multi-step sorting strategies to isolate CAR T cells with desirable properties. To process the resulting material, the inventors have developed protocols for cell fixation, de-crosslinking for efficient genomic DNA (gDNA) isolation, single-step gRNA amplification, and next-generation sequencing. Finally, the feasibility of genome-wide screens with proliferation and sorting readout is shown, demonstrating that the employed system is highly scalable and efficient. Finally, the inventors demonstrate that the present workflow supports pooled in vivo screens in mouse tumor models.

### Plasmids and Plasmid Libraries

Basic Constructs Encoding the CAR and a CRISPR gRNA Cassette

The present invention comprises a series of lentiviral transfer plasmids encoding a chimeric antigen receptor (CAR) and CRISPR guide RNA (gRNA) expression cassette, which are part of the invention and enable powerful and effective optimization screens. The gRNA is cloned into the 3′ LTR, making the system compatible with the CROP-seq technology for screens in single cells¹. CARs are expressed under the EF1α promoter, and composed of a leader peptide, single-chain variable fragment (scFv) antibody binding CD19 (clone FMC63 or SJ25C1), the hinge and transmembrane domains of CD8, intracellular co-stimulatory domains (CD28 or 41BB) and the CD3ζ chain. In total 21 such plasmids were constructed, using different combinations of these building blocks. Upon successful delivery of the constructs, T cells express the chimeric antigen receptor on their surface (FIG. 2a), and mediate cytotoxic killing of CD19-expressing tumor cell lines in vitro and control tumor growth in a systemic leukemia model as described in detail below.

According to the inventor's knowledge, there are currently two papers describing genome-wide CRISPR screens in human primary T cells²³ and a single example of a screen done directly on CAR T cells⁴. Compared to these reports, the inventor's plasmid design has one major advantage: for the first time they combine the CAR and CRISPR gRNA on the same vector. This turned out to be an important design decision for efficient screening, since it ensures that each functional CAR T cell also contains a CRISPR perturbation. The present approach is much more efficient than using multiple lentiviruses in succession, as for example done in 3 and 4. Using CAMEL, a much higher number of T cells can be modified, which allows higher quality screens with larger libraries, deeper coverage, and/or multiple functional readouts.

Constructs Encoding gRNA Libraries or Single gRNAs

Most importantly, the inventors have derived versions of the above plasmids that encode CRISPR gRNA libraries. These libraries were PCR-amplified from custom-designed oligo pools or existing plasmid libraries, and cloned by Gibson's isothermal assembly. They include small-scale libraries targeting selected control genes, libraries targeting curated T cell genes, a small-scale base editing library and genome-wide libraries targeting each human gene with four gRNAs. In addition, the inventors derived single gRNA expressing constructs and established flow cytometric assays for genome editing of the surface markers CD25 and CD44. These are used for protocol development and to monitor the editing efficiency during the screen.

### Reporter Constructs and Engineered Cell Lines

To functionally test CAR T cells both in vitro and in vivo, the inventors have generated a number of plasmid constructs and used them to establish stable cell lines and functional assays. To support bioluminescence imaging, the inventors constructed a transgene of firefly luciferase, the resistance gene for the antibiotic zeocin and the fluorescent reporter mPlum. The inventors used these constructs to establish stable lines from the B cell precursor leukemia cell line NALM6 that expresses endogenous levels of CD19. The inventors established plate-based high-throughput assays of CAR T cell killing activity based on firefly luciferase (FIG. 2b) and a systemic leukemia model in NSG mice as described below. To further investigate the effect of surface antigen density, the inventors used CRISPR to induce indels in the CD19 coding sequence, and isolated heterozygous mutants by clonal expansion. The resulting cell lines show a characteristic, reduced expression level of CD19 (FIG. 2c) and can be used to test CAR T cells in the context of antigen downregulation.

Furthermore, the inventors cloned constructs encoding the antigen recognized by the CAR (e.g., CD19) or an unspecific negative control (e.g., CD20) in fusion with a GFP reporter. The inventors used these constructs on K562 cells, which do not express any endogenous levels of CD19 or CD20, to use them as target cells in the present co-culture system, as described in detail below.

### Optimized Workflow for T Cell Modification

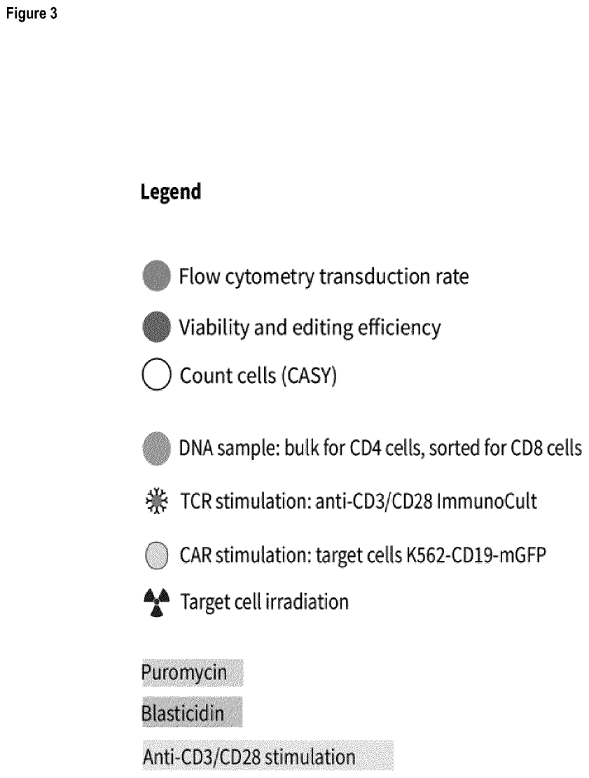

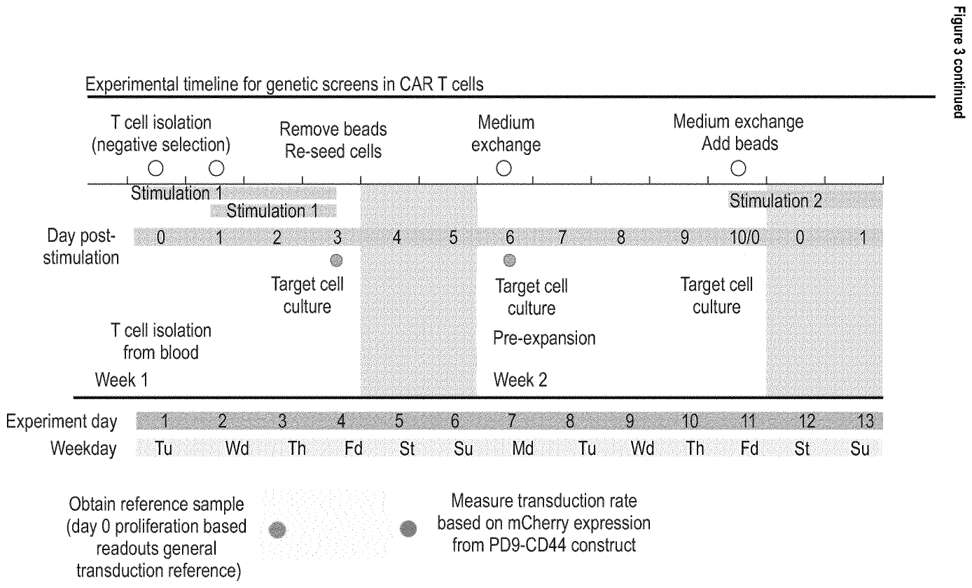

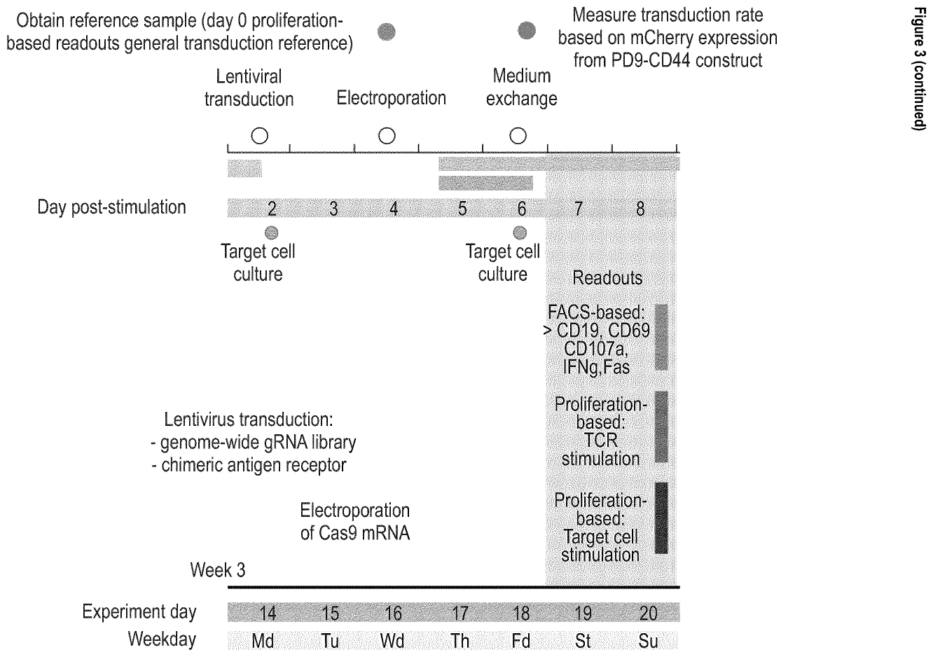

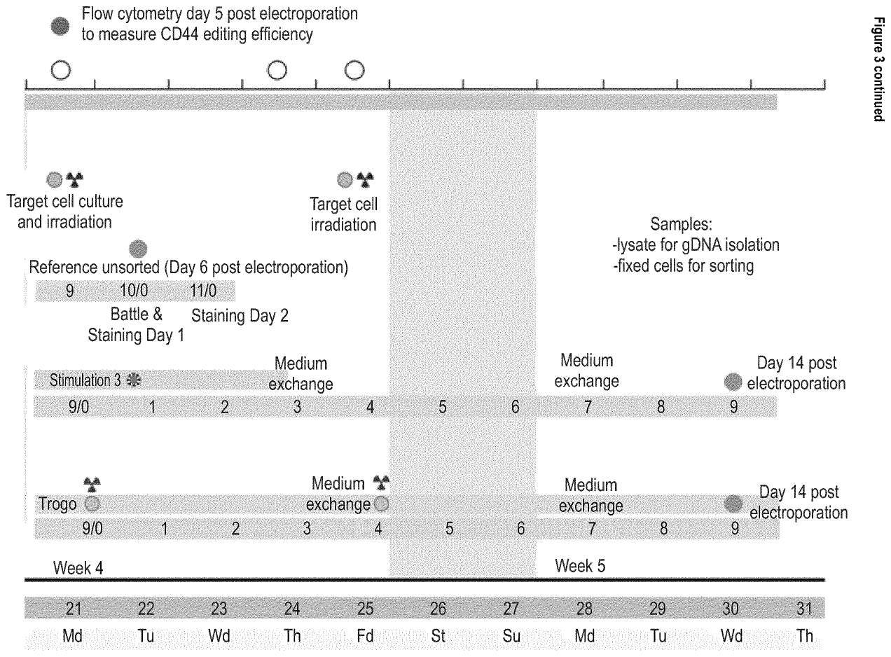

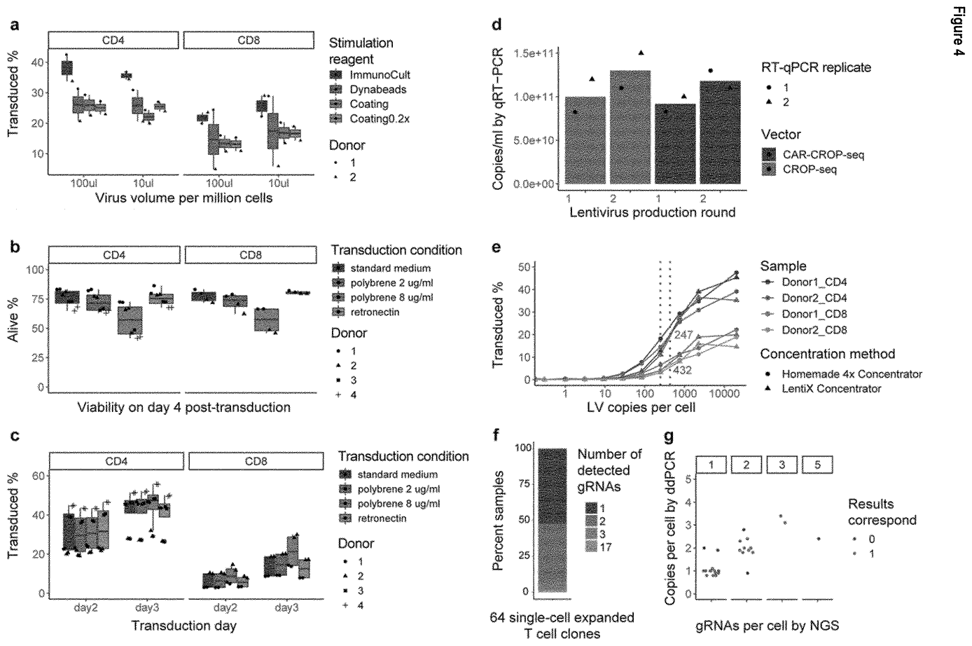

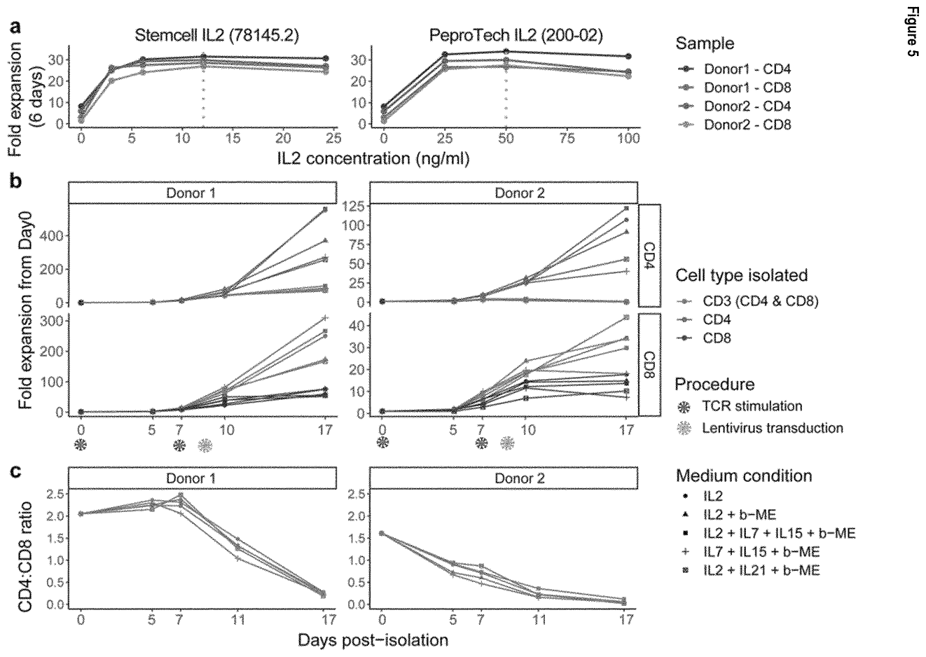

It is well established that the efficiency of T cell modification depends on their stimulation status, the culture conditions and their surface density. The present invention includes a fully optimized, scalable, and highly efficient laboratory workflow for genetic screens in CAR T cells. A typical screen follows the experimental timeline in FIG. 3. Human primary CD4+ or CD3+ T cells are isolated from peripheral blood donations by negative magnetic selection. Immediately upon isolation, T cells are activated by TCR stimulation and co-stimulation via anti-CD3/anti-CD28 activator reagents. T cells are cultured in OpTmizer medium with 2% human serum and supplemented with IL2. To obtain sufficient cells for genome-wide screening, cells undergo an initial expansion period of 9-10 days, and are then re-stimulated. On day 2 post stimulation, cells are transduced with a defined amount of highly concentrated lentivirus encoding the CAR and a gRNA library. On day 4 post stimulation, cells are electroporated with custom-made mRNA encoding a CRISPR modifier and selection marker (e.g., encoding BSD for blasticidin resistance). This step initiates the genome editing process. At day 5 or 6 post stimulation, cells are selected with both puromycin (to select for the CAR/gRNA construct) and blasticidin (to select for successfully electroporated cells). The puromycin selection is then maintained until the end of the experiment. Functional readouts are ideally done on days 5-6 post electroporation, to allow for efficient cell engineering and selection of modified cells.

As part of this work, the inventors have intensely studied T cell transduction. This has allowed them to derive methods for the titration and exact transduction of human primary T cells, which result in consistent transduction rates across many lentivirus production rounds and T cell donors.

Importantly, the optimization data suggests that in a culture of primary human T cells, only a subpopulation of cells is amenable to lentivirus transduction. This is demonstrated by T cell transduction with high lentivirus concentrations, where a 10-fold difference between virus volumes does not lead to increased transduction efficiency (FIG. 4a). One likely explanation is that only a fraction of cells is fully activated by the stimulation beads, leading to proliferation and changes in their membrane composition that facilitate viral entry. This hypothesis is supported by varying transduction efficiencies in cells treated with different TCR stimulation agents, among which ImmunoCult CD3/CD28 Activator (STEMCELL Technologies) led to the highest transduction rates and therefore was chosen for genetic screens (FIG. 4a). In the process of further optimization of transduction conditions, the inventors have found that commonly used reagents such as polybrene are toxic to T cells (FIG. 4b) and that frequently used methods such as spinfection and plate coating with retronectin do not further improve T cell transduction (FIG. 4c). Furthermore, the inventors showed that transduction is optimal on day 3 for CD8 cells (FIG. 4c).

Transduction rates in T cells saturate in the range of 10-50% (FIG. 4a), but increasing lentivirus amounts may introduce multiple copies of the construct. In a CRISPR screen with a gRNA library, each cell should ideally receive only one gRNA. Multiple integrations would result in a random set of genetic perturbations per cell. The inventor's method quantifies the lentivirus titer with a qPCR-based assay, which reproducibly identifies the number of lentivirus genome copies per volume unit (FIG. 4d). Then a standardized number of virus copies per cell is used (247 copies per cell for screens in CD4 T cells and 432 copies per cell for screens in CD8 T cells). This typically results in transduction rates of ~15% (CD4) or ~10% (CD8) (FIG. 4e). By expansion of single cell clones followed by gRNA sequencing, the inventors showed that this approach results in an average number of 1.6 gRNA integrations per successfully transduced cell (FIG. 4f). In addition, the inventors have established a protocol based on digital droplet PCR (ddPCR) that simplifies the verification of integration number in antibiotic-selected cells and correlates well with gRNA sequencing-based counting of integrations in single cell expanded clones (FIG. 4g). In a genome-wide screen, most perturbations will not have an effect on the phenotype. Thus, the inventors have chosen a concentration where some cells still have double integrations, to maximize the number of T cells that are transduced for the screen.

### Optimized Workflow for T Cell Culture

Large cell numbers are required to maintain high gRNA coverage at every step of a genetic screen. To obtain maximum T cell numbers, the inventors extensively optimized cell culture conditions. IL2 is the major supplement that supports T cell growth. We titrated IL2 to achieve maximum expansion, and found optimal concentrations for two IL2 reagents (FIG. 5a). It is also shown that the IL2 addition is sufficient for proliferation, while other frequently used supplements, such as IL7, IL15, IL21, beta-mercaptoethanol (b-ME) did not further improve T cell expansion (FIG. 5b).

The inventors surprisingly found that CD8 T cells do not sufficiently expand for genome-wide screens when cultured alone, but proliferate well in the presence of CD4 helper cells (FIG. 5b). Thus, the inventors have developed a novel co-culture method for effective proliferation of CD8 T cells in genome-wide CRISPR screens. At the same time, the inventors noticed that CD4 cells alone have a much greater proliferation potential than in mixture with CD8 cells, where the CD4:CD8 ratio declines over time (FIG. 5c). The present work demonstrates for the first time that genome-wide screens in CD4 cells are only feasible when cultured alone, and that screens in CD8 cells require the isolation of pan-CD3 cells, where CD8 cells will eventually grow out.

The inventors have found empirically that the density of T cells and CAR T cells on the surface of culture vessels has an influence on their transduction, electroporation, and target cell killing. The inventors could define which cell numbers, culture vessels and seeding densities are optimal.

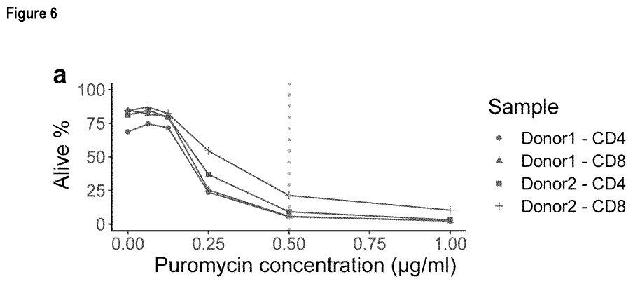

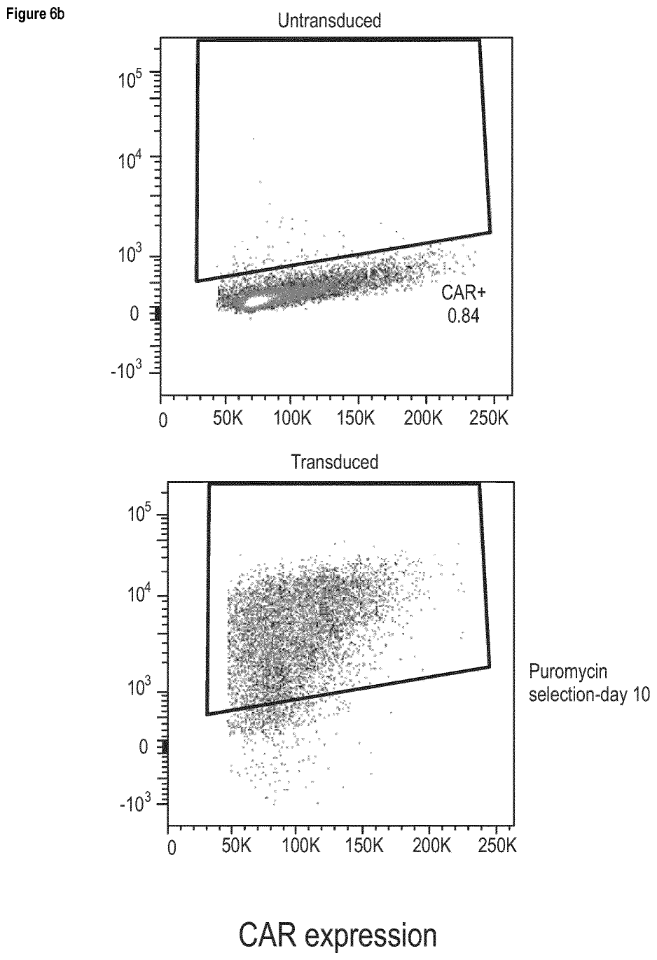

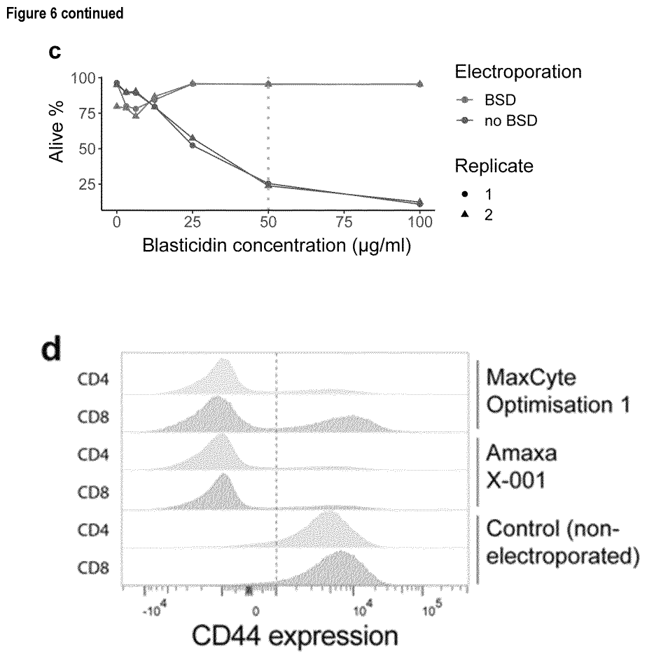

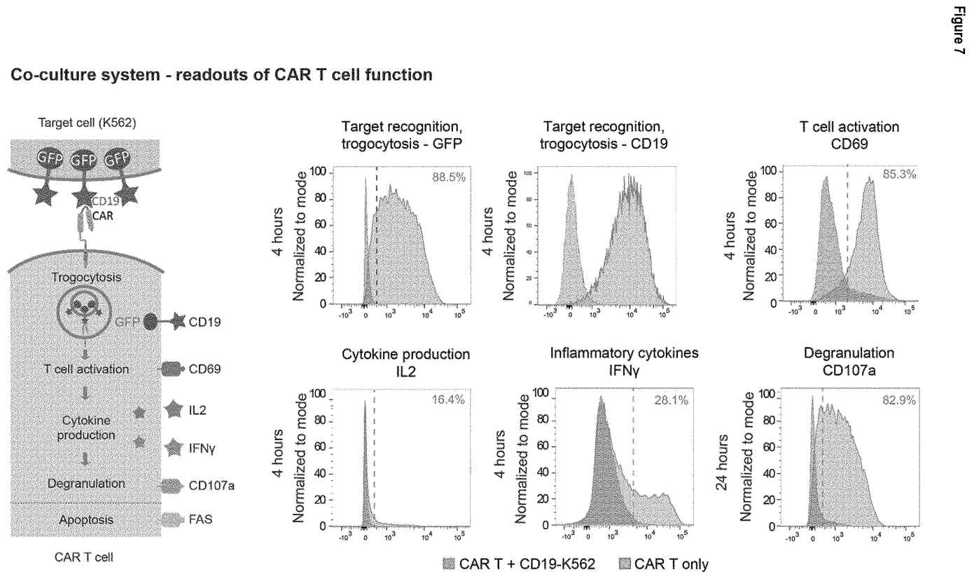

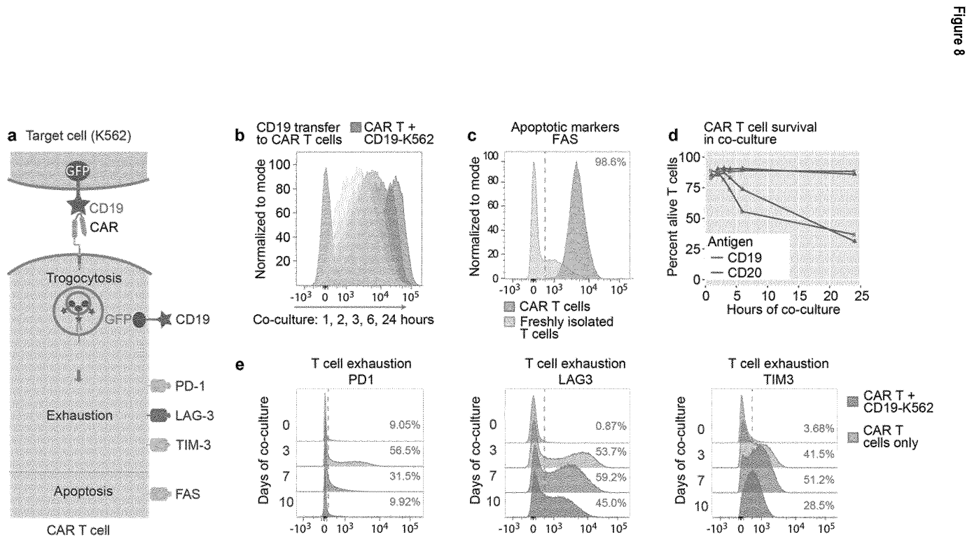

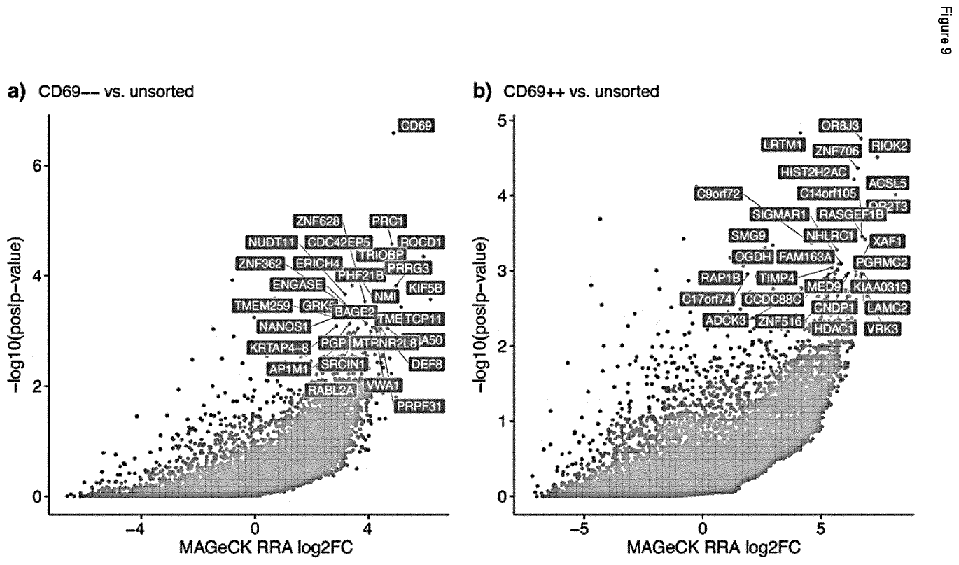

For genetic screens, the inventors carefully optimized antibiotic concentrations to select for successfully transduced and electroporated cells. A puromycin concentration of 0.5 μg/ml leads to >75% death of untransduced cells within 1 week of selection (FIG. 6a), and >90% selection of CAR-positive cells within 10 days (FIG. 6b). Depending on the timepoint of the readout, a minor fraction of untransduced cells may remain in the culture. However, this will not affect the pooled screening results, as untransduced cells do not contain a gRNA. Efficient antibiotic selection is nevertheless critical to minimize cell culture expenses and is generally superior to FACS, which is more damaging to cells and limiting for large screens. At the same time, selection of electroporated cells can substantially improve screening results by removing cells that did not express a CRISPR modifier and therefore did not receive a genetic modification. To select edited cells, the present method uses co-electroporation of BSD mRNA together with CRISPR editor mRNA. One day post electroporation, cells are treated with blasticidin at a concentration of 50 μg/ml for a duration of 1-2 days, which leads to >85% death of non-electroporated cells (FIG. 6c). Using this approach, 75-90% editing can be achieved on two different electroporators (Amaxa Nucleofector I and MaxCyte ATX) with optimized programs (FIG. 6d).

### Co-Culture System to Optimize CAR T Cells for Immunotherapy

The key to a successful screen is an efficient way to isolate populations of interest from a pool of modified cells. The gRNAs in each population can then be counted by next-generation sequencing, to find CRISPR perturbations that are enriched or depleted in biological phenotypes. To isolate CAR T cells with desirable properties for immunotherapies, the inventors developed an innovative co-culture system with target cells. For this system, the inventors engineered cancer cell lines to express the antigen recognized by the CAR on their surface (e.g. human CD19), in fusion with a reporter gene such as GFP. As a negative control, target cells can instead be engineered with an unspecific antigen not bound by the CAR (such as human CD20) that is fused to the same reporter.

Characterizing this system in detail (FIG. 7, FIG. 8), the inventors observed that when functional T cells engage with their target, the T cell receptor (TCR) pathway is activated and the TCR gets internalized. This also results in the uptake of the antigen and parts of the target cell membrane by T cells, a process known as trogocytosis. In the employed system, the inventors found that both the antigen and reporter are readily detected on the surface and inside of functional CAR T cells when they engage with their targets. The present invention establishes for the first time that trogocytosis can be effectively used as a screening readout, using GFP or CD19 staining as markers. This readout can be used alone, to identify CAR T cells that have successfully engaged with their targets. However, it is just as powerful when used as a filter for downstream readouts that the inventors have developed. For example, CAR T cells that have successfully recognized their target, but do not show any downstream signaling (CD69), degranulation (CD107a), or cytokine production (IFNγ) (FIG. 7) can be isolated.

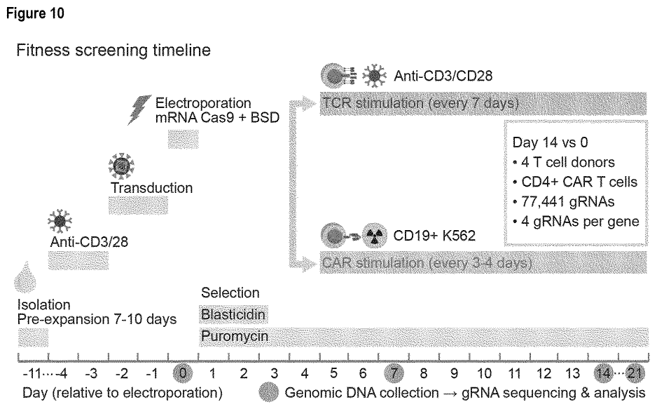

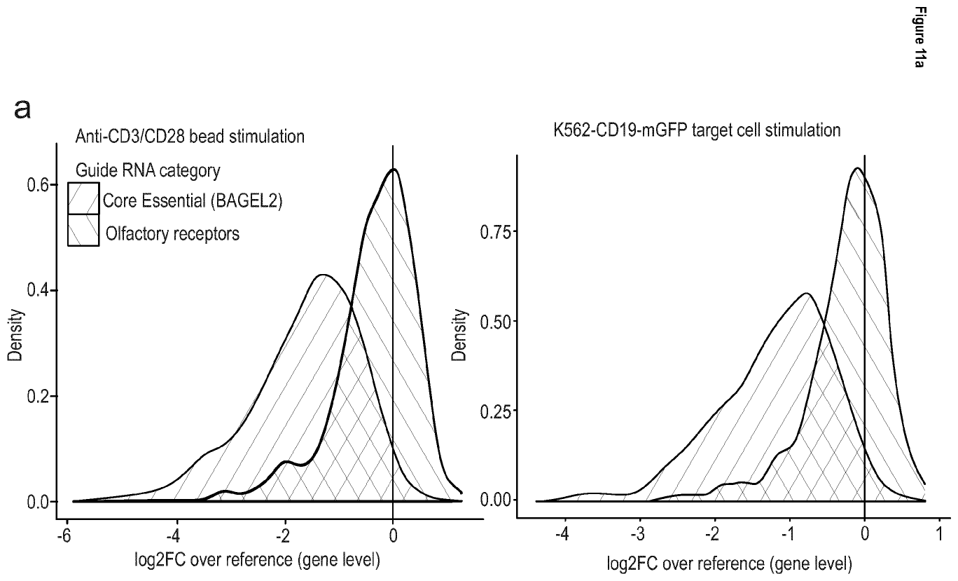

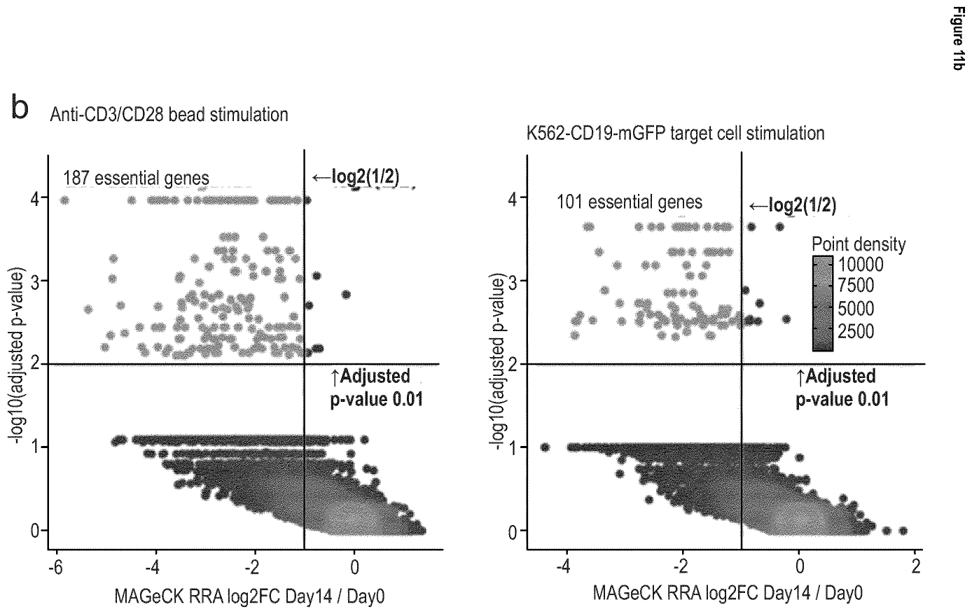

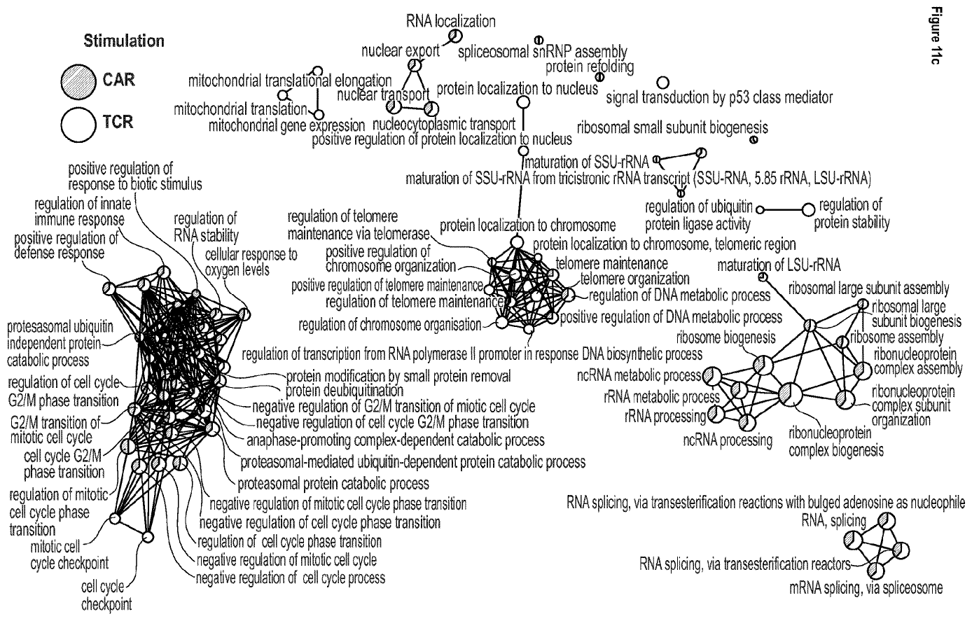

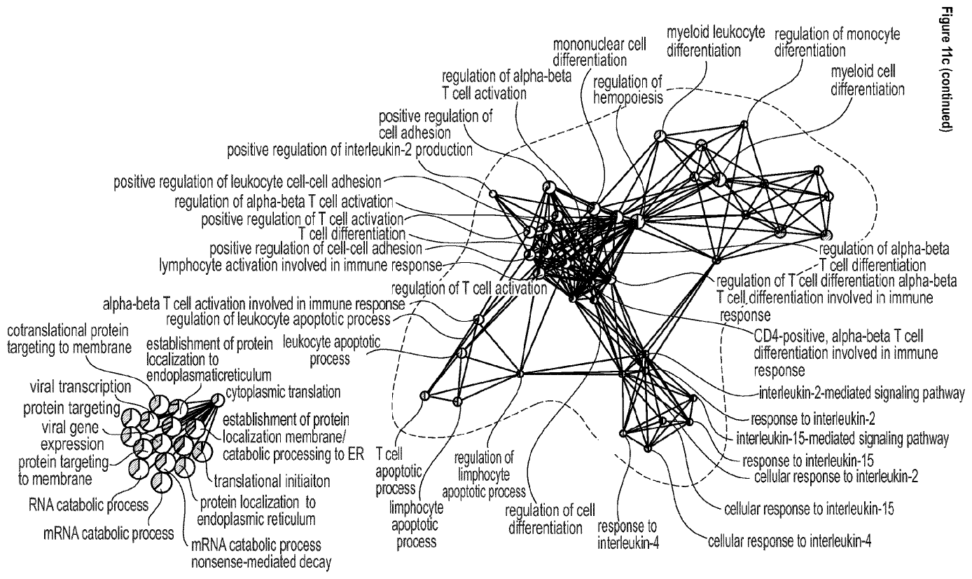

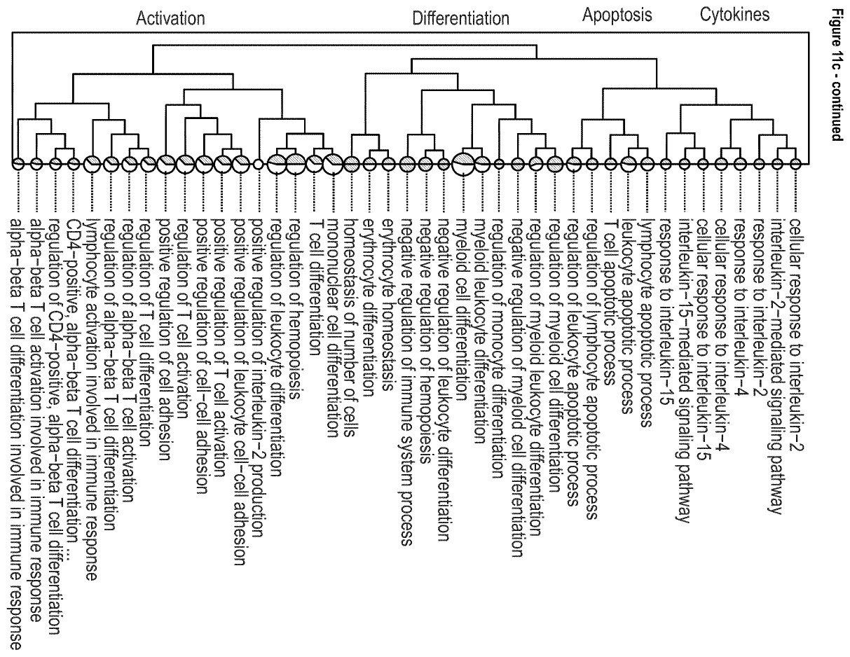

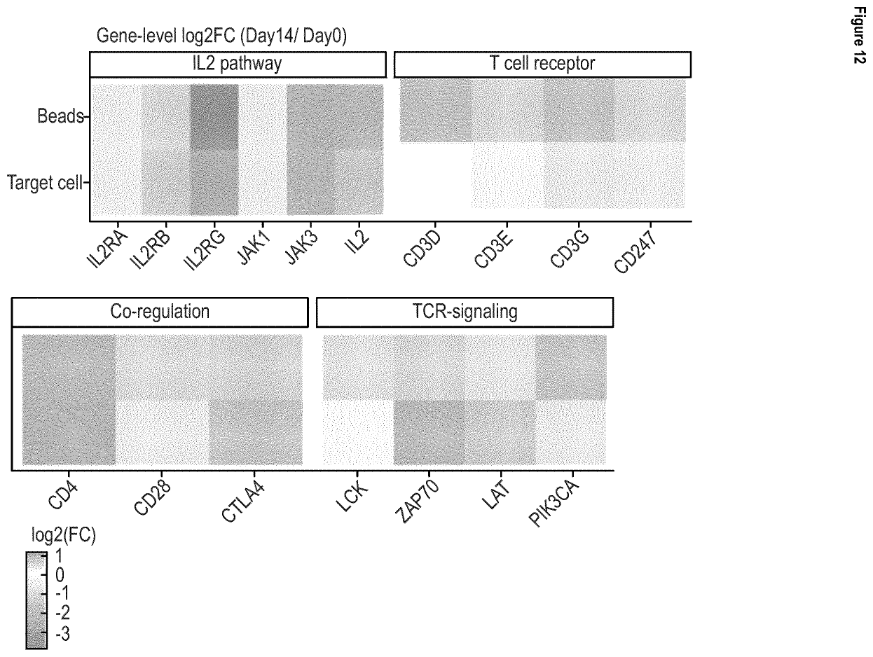

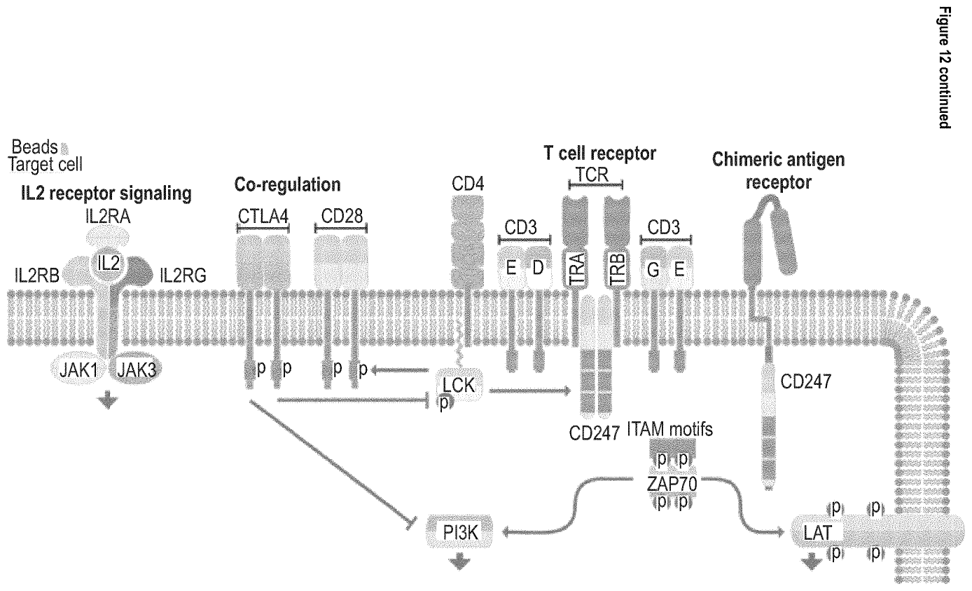

Another big limitation of CAR T cell immunotherapies is that the uptake of CD19 by T cells through trogocytosis makes them a target for killing by other T cells, a phenomenon referred to as T cell fratricide⁵. The inventors also found that prolonged in vitro culture leads to the upregulation of the death receptor Fas, which renders CAR T cells susceptible to apoptosis and fratricide. As the result, in the employed system roughly two thirds of T cells die within the first 24 hours of co-culture (FIG. 8). The remaining one third of cells expand, but acquire a T cell exhaustion phenotype characterized by the expression of PD1, LAG3 and TIM3. T cell exhaustion has been associated with a poor clinical performance of CAR T cells. Thus, the present co-culture system addresses key clinical limitations of CAR T cell therapies, which can be addressed in sorting- or proliferation-based screens. For example, it is possible to FACS-purify CAR T cells that are GFP+ (have internalized the CAR for efficient signaling), but do not present CD19 on their surface. Alternatively, it is possible to screen for genes with a role in T cell trogocytosis, fratricide, exhaustion and proliferation through the experimental design outlined in FIG. 10, where CAR T cells are stimulated by either the TCR or CAR, and the gRNA dynamics over time are later compared.

### Examples of Successful Sorting- and Proliferation-Based Screens

To date, only a small number of genetic screens in primary human T cells have been described, and there is a single, low-quality example of a screen done directly on CAR T cells.

All of these screens employed positive FACS-based selection to enrich for populations of interest. This type of positive-selection screen can compensate for low genome engineering efficiencies by selecting phenotypes not typically found in the wild type population. For example, experimenters can sort very high levels of a marker, assuming that the upregulation is due to an effective genetic perturbation and unedited cells are excluded. CAMEL has important advantages over state-of-the-art methods. First, it is less damaging and maintains cells in a highly proliferative state. Second, CAMEL yield a much greater number of T cells for screening, so that researchers can achieve a higher library complexity, perform more readouts or both. This is evident from the present work, where the inventors routinely perform 8 genome-wide readouts from one T cell sample while maintaining a library complexity of >2000× per readout. The inventors included an example of a successful sorting-based screen in FIG. 9. In this screen, the inventors used CAMEL to establish a genome-wide pool of knockout CAR T cells, and took an initial unsorted sample. The inventors then combined the modified CAR T cells with target cells expressing the antigen CD19. This leads to target recognition, and the upregulation of the early activation marker CD69 in functional CAR T cells. At four hours, cells were stained for CD69 (among other markers), fixed and separated by FACS into CD69−− (lowest 7%) and CD69++ (top 3.5%). Based on log 2 fold changes between the CD69−− and unsorted populations, gRNAs targeting CD69 were identified as the top hits (FIG. 9a), highlighting the efficiency and specificity of our system. Next, the inventors compared the CD69++ and unsorted populations, which is particularly relevant for optimizing immunotherapies. The inventors identified several genes that result in unusually high levels of CD69, indicating that they have a particularly high sensitivity or enhanced response to target cell stimulation (FIG. 9b). To give an example, ACSL5 was identified in the screen. The role of this gene in primary human T cells has so far not been described. Based on the literature, ACSL5 expression is higher in systemic lupus erythematosus patients compared to healthy controls⁶. In the Jurkat T cell line, ACSL5 expression was increased during apoptosis induced by PMA-ionomycin, a very strong stimulus for TCR activation and siRNA downregulation of ACSL5 decreased the induced apoptosis⁷. The data suggests a role for ACSL5 in CAR T cell apoptosis, and that knockout in ACSL5 in CAR T cells could potentially make them more resistant to overactivation-induced apoptosis.

Proliferation-based readouts, where gRNAs encoding certain genetic perturbations drop out over time while others are enriched are hard to perform in a quantitative manner. Since the goal is to identify gRNAs that are depleted from a population, they suffer particularly from low editing efficiencies, low cell proliferation rates and noise associated with low library complexity. To the best of the inventors' knowledge, the present work represents the first ever proliferation-based screen in primary human T cells. In FIG. 10-FIG. 13, summarize key data from a successful genome-wide dropout screen are summarized. For this experiment, the inventors followed the experimental design in FIG. 10. Proliferation-based readouts are particularly important to identify and understand mechanisms that are essential for CAR T cells. Such mechanisms can then be enhanced, e.g. by overexpression of ORFs to improve CAR T cells.

The inventors first transduced T cells with a lentiviral vector encoding the CAR and a genome-wide gRNA library and took a Day 0 sample. The inventors then electroporated cells with Cas9 mRNA to induce genome editing and selected with antibiotics to enrich for modified cells. The inventors then split the sample into two parts, the first was stimulated via the TCR with anti-CD3/CD28 stimulation beads, and the second was stimulated via the CAR with K562-CD19-mGFP target cells. The cultures were sampled at Day 14 post electroporation.

Over time, the inventors observed a depletion of essential genes compared to non-essential olfactory receptors widely used as a control (FIG. 11a). Using stringent cutoffs, the inventors selected statistically significant essential genes and performed Gene Ontology (GO) enrichment analysis (FIG. 11b). The inventors then clustered the top 100 enriched GO terms based on the GO term hierarchy. This way, the inventors identified major essential processes such as the cell cycle, transcription and translation, splicing, and ribosome biogenesis, but also a cluster of T cell specific processes (FIG. 11c). The latter includes IL2 signaling, and T cell activation, differentiation, and apoptosis.

Next, the inventors calculated log 2 fold changes for selected genes and mapped them onto a schematic of the T cell receptor pathway (FIG. 12). Both bead and target cell stimulated samples strictly required signaling through the IL2 receptor for their proliferation, in particular the genes IL2RG and JAK3. IL2 is the main cytokine required for T cell proliferation, and therefore used in all culture protocols. In addition, the co-receptor CD4 was essential for signaling via the TCR and CAR. The present system also captures more subtle aspects of TCR signaling, such as co-regulation. For example, the positive co-receptor CD28 was depleted at Day 14, while its negative counterpart CTLA4 was enriched. We also found interesting differences between TCR and CAR stimulated samples. As expected, the TCR subunits CD3E and CD3D were essential only in TCR-stimulated samples, where they are activated by the bead-bound anti-CD3 antibody. The inventors have further unexpectedly found that knockouts of the kinase ZAP70 and the adapter protein LAT did not abolish signaling in CAR T cells, but rather resulted in increased proliferative potential. Perhaps, a knockout of ZAP70 or LAT reduces overactivation of CAR T cells, which can result in exhaustion and cell death.

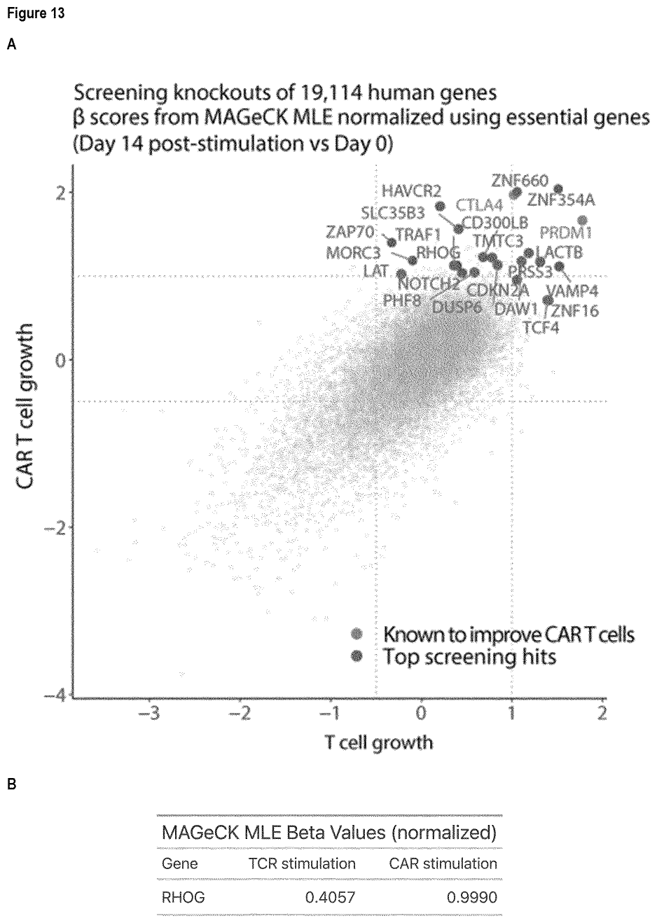

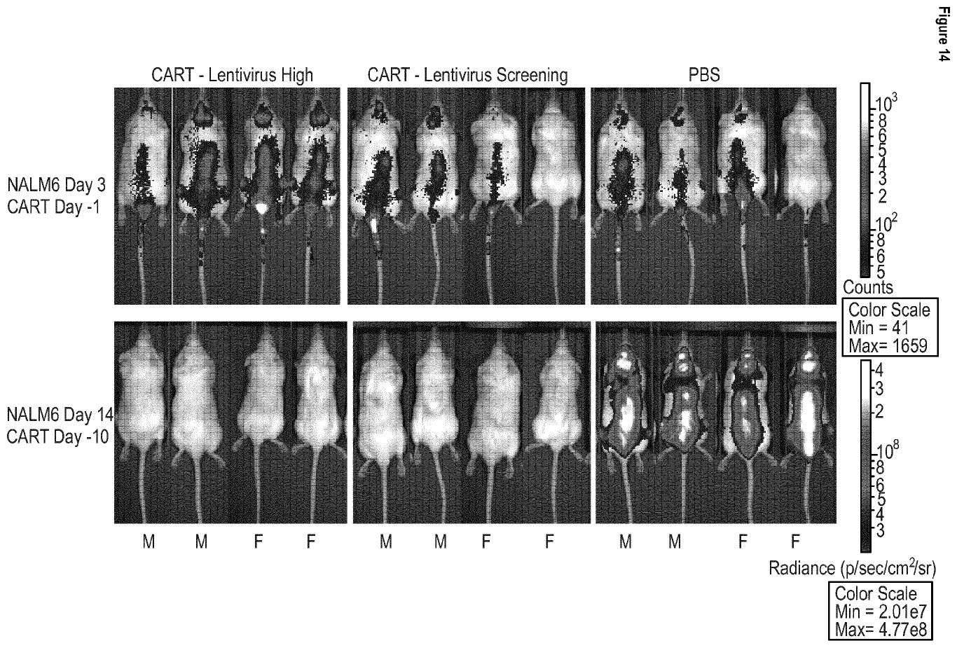

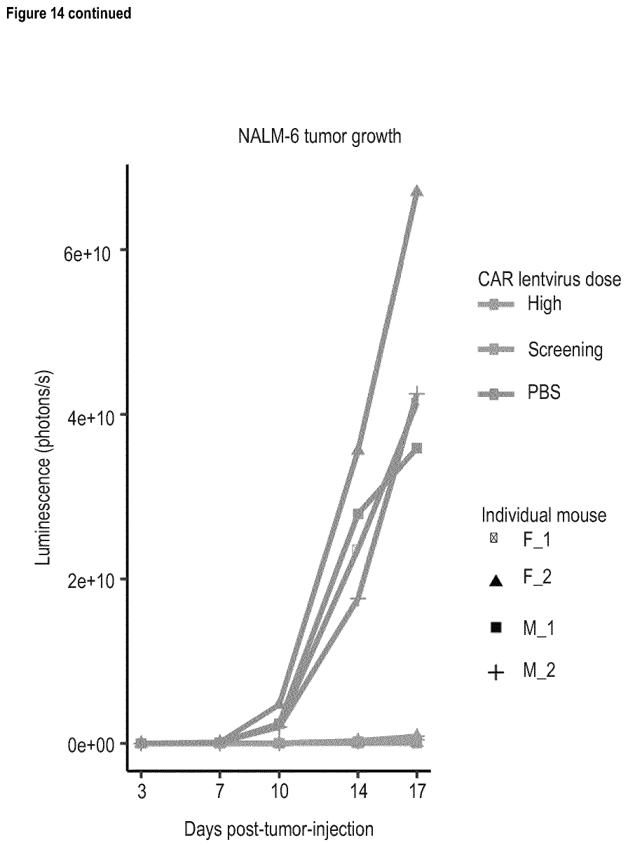

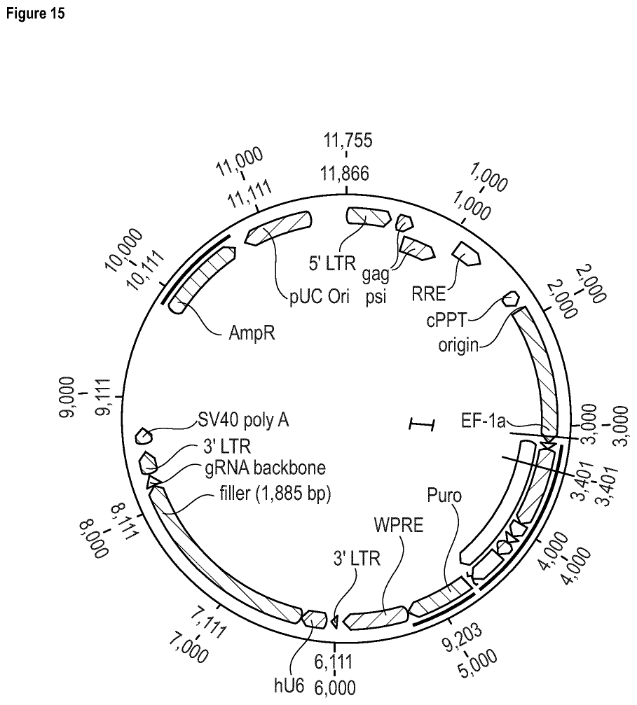

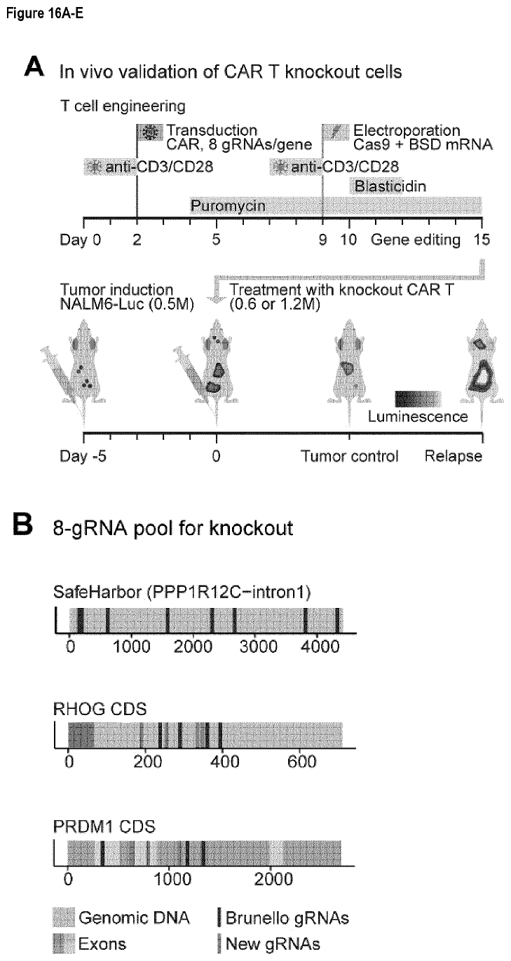

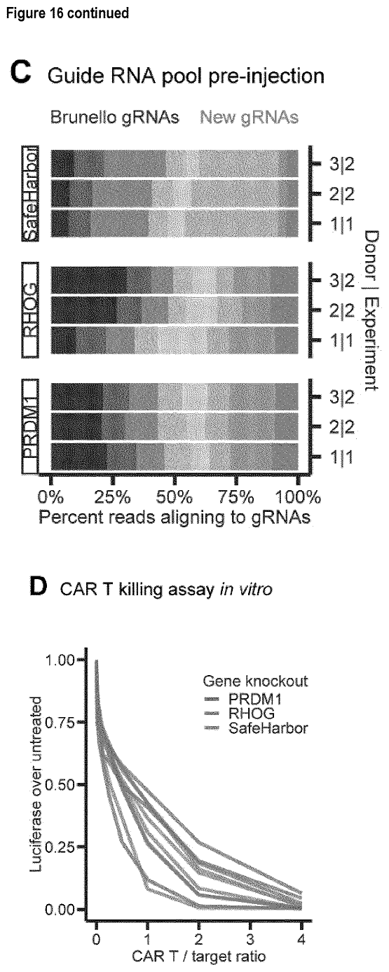

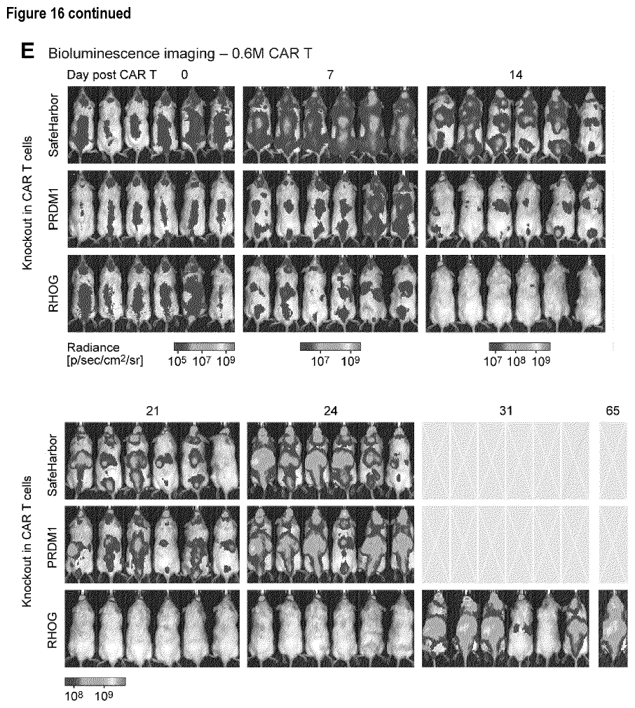

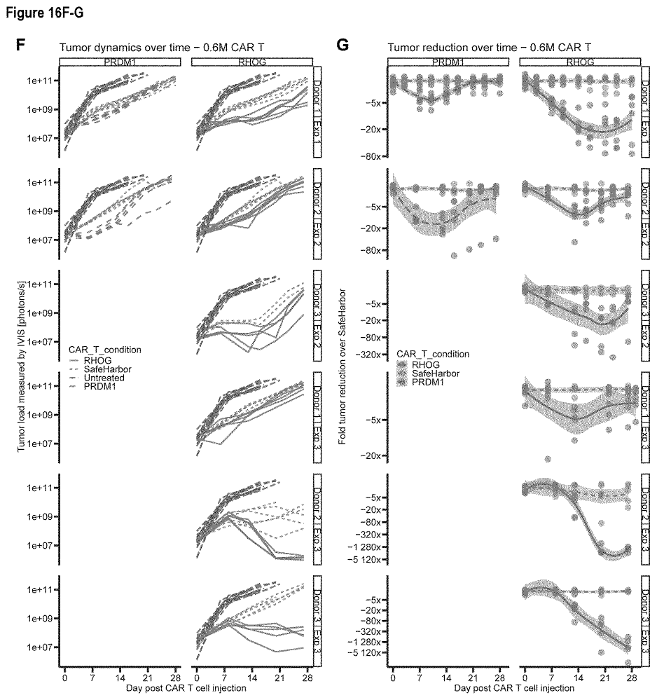

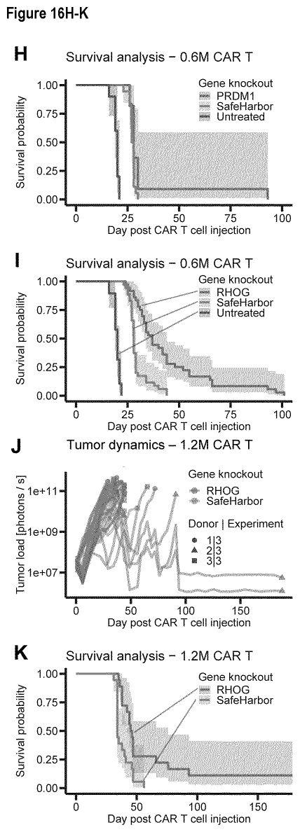

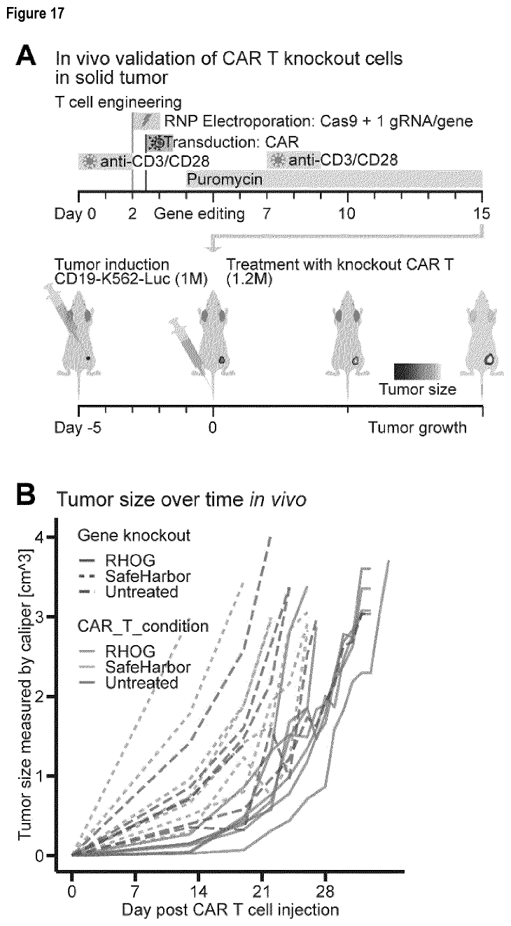

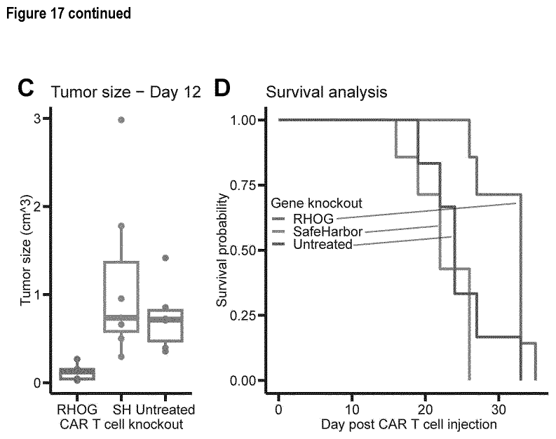

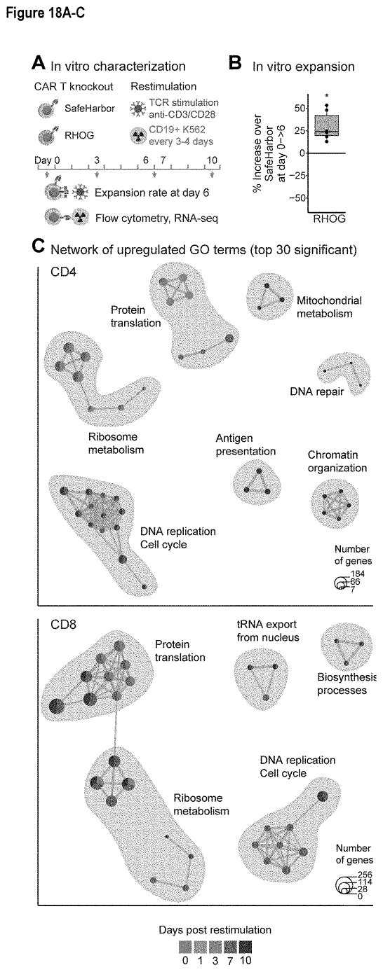

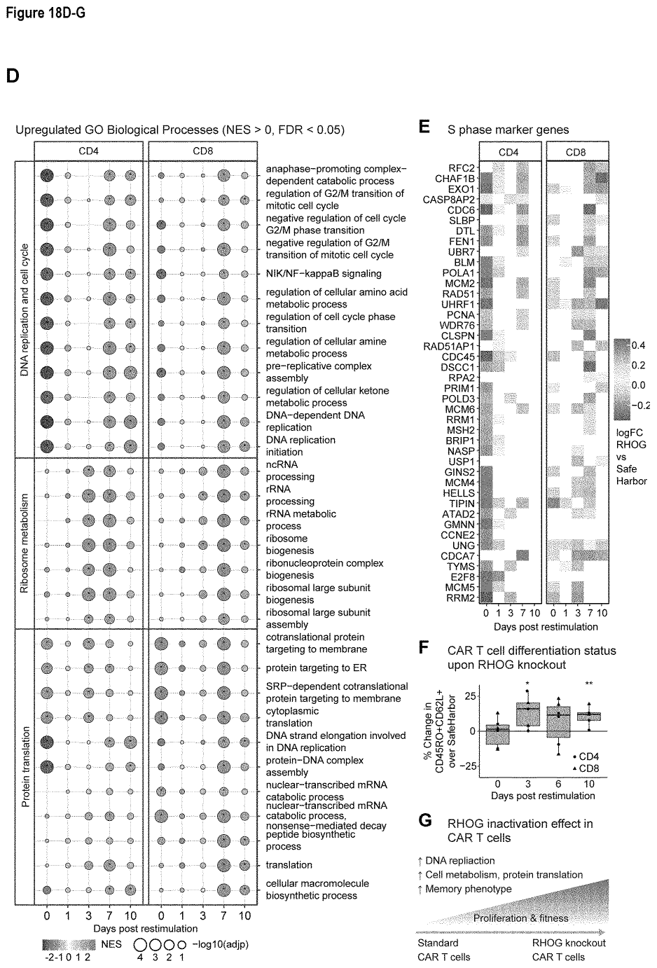

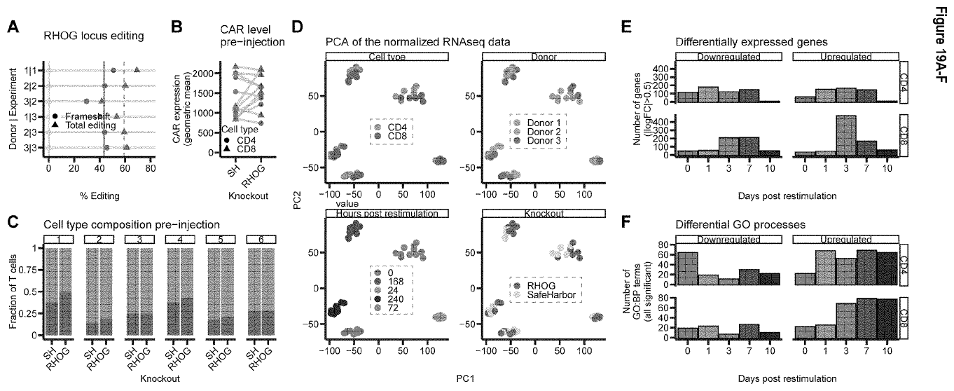

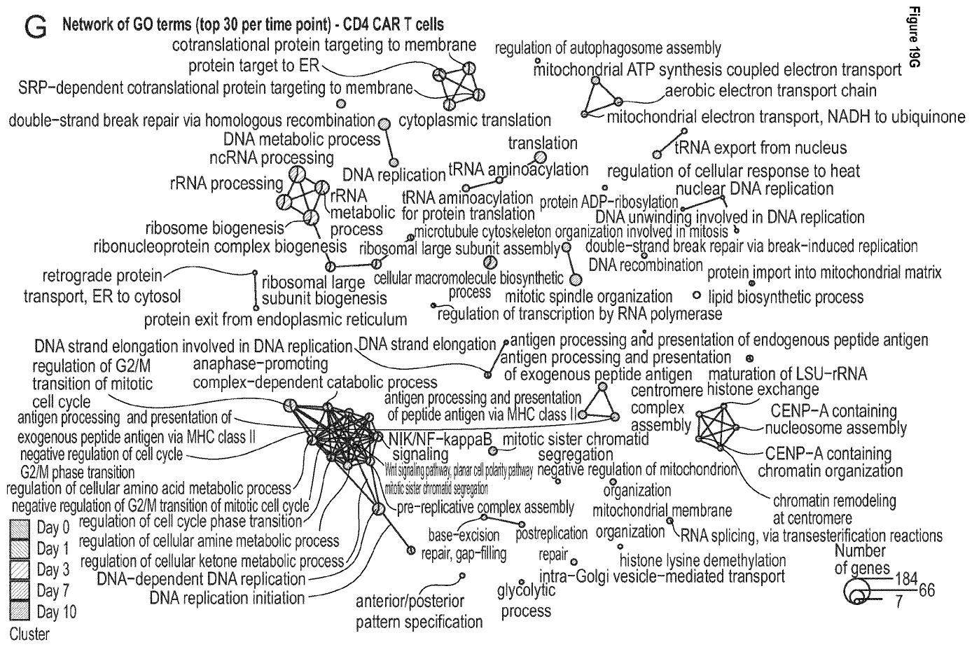

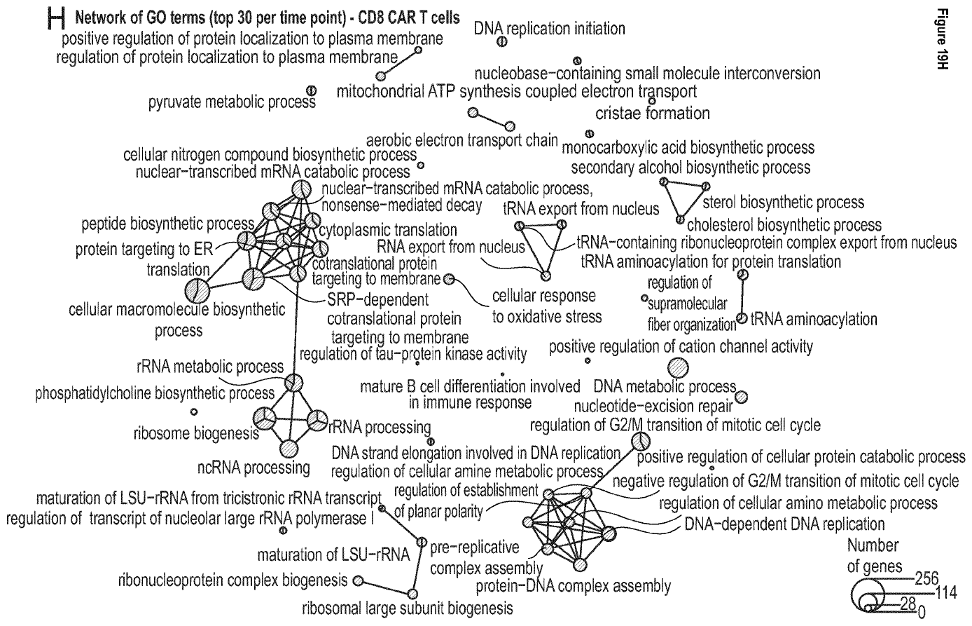

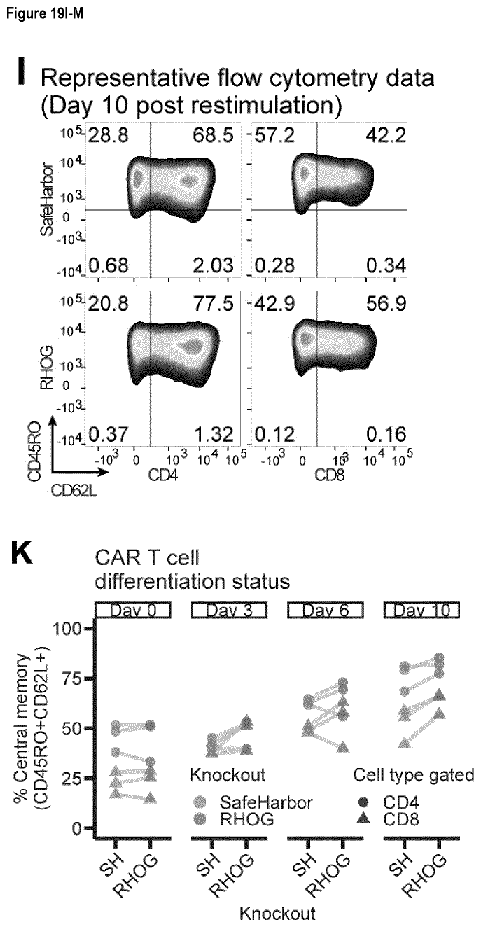

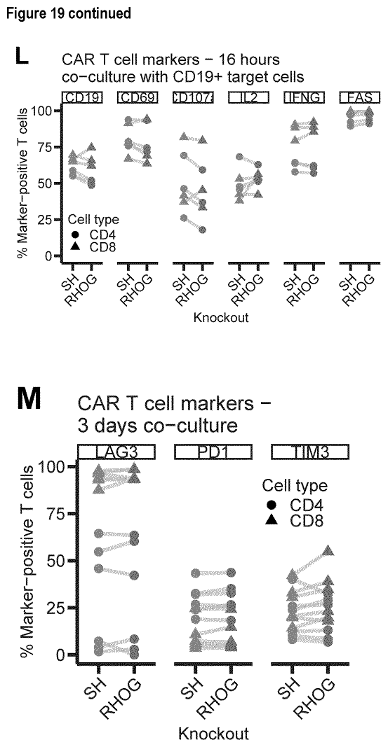

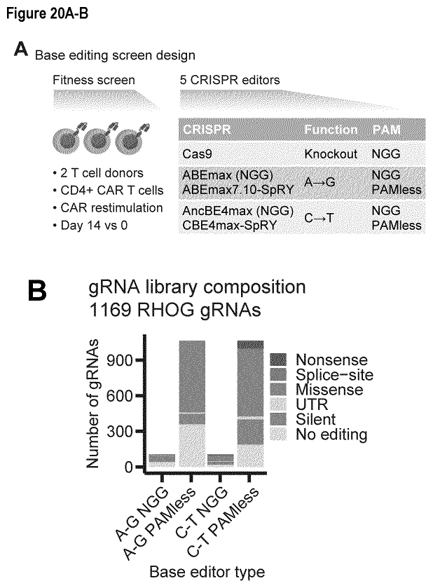

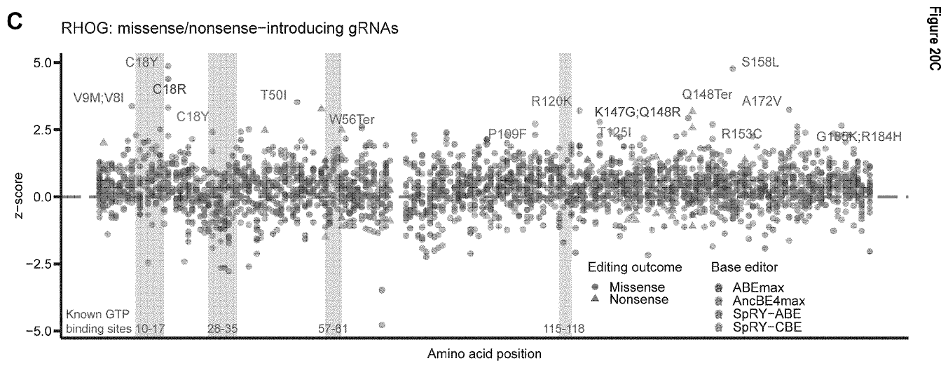

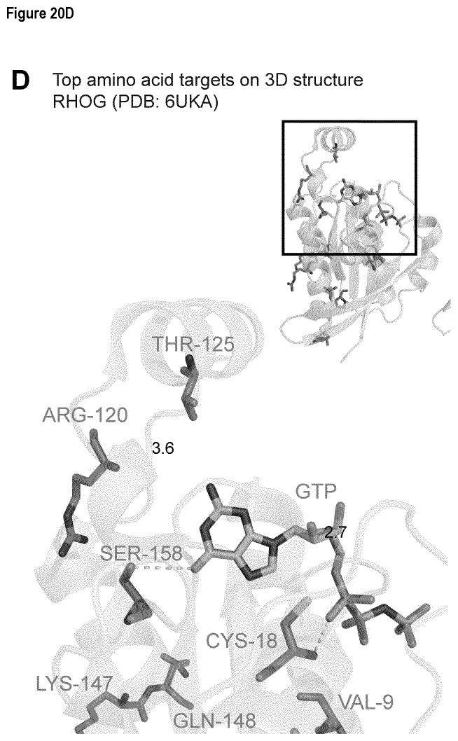

Finally, the inventors set out to discover new genes, the deletion of which could improve (CAR) T cell therapies. The inventors processed the screening data with the MAGeCK MLE algorithm, which yields beta values that are more representative of the effect size of genetic perturbations. The inventors normalized the beta values based on core essential genes, and compared them between TCR and CAR stimulated samples (FIG. 13). This way it was possible to select knockouts that specifically promote CAR T cell proliferation (Table 3), and knockouts that more generally promote both T and CAR cell expansion (Table 4). Both gene sets are of great interest to improve T cell-based therapies. Gene knockouts that promote CAR T cell proliferation particularly strong are shown in Table 5.

### Workflow for In Vivo Screens

CAR T cells for clinical or preclinical models are typically produced with excessive amounts of lentivirus to ensure a high transduction efficiency. This results in multiple CAR integrations per cell, higher CAR expression levels and a greater antigen sensitivity in vivo. Genetic screens, however, require a much lower lentivirus concentration to achieve a single integration and defined genetic perturbation in each cell. The inventors used a mouse tumor model induced by NALM6 cell injection to compare treatment effects by CAR T cells produced with our optimized lentivirus concentration for single gRNA integrations, or a 30× higher lentivirus concentration. Despite a potential disadvantage for CAR expression, the inventors observed efficient tumor clearance in both conditions (FIG. 14), showing that our platform supports pooled in vivo screens.

### Example 2. RHOG Depletion or Inactivation Enhances T Cell Function

Materials and methods employed for generating the data described in the present Example 2 are described in detail further below.

### Genome-Wide Proliferation Screens in CAR T Cells Stimulated Via CAR or Via T Cell Receptor Identifies RHOG as an Immunotherapy Target

As described in Example 1, the inventors processed the screening data with the MAGeCK MLE algorithm (Li, Genome Biol. 2015 Dec. 16:16:281), which yields beta values that are representative of the effect size of genetic perturbations. We normalized the beta values based on core essential genes (Kim, Genome Med. 2021 Jan. 6; 13(1):2) using the MAGeCKFlute package (Wang, Nat Protoc. 2019 March; 14(3):756-780), and compared them between TCR and CAR stimulated samples (FIG. 13). This way it is possible to select knockouts that specifically promote CAR T cell proliferation, and knockouts that more generally promote both T and CAR cell expansion. Both gene sets are of great interest to improve T cell-based therapies.

### RHOG Knockout Enhances In Vivo Tumor Control by CAR T Cells in a Leukemia Xenograft Model

To assess the performance of single CAR T cell modifications in vivo, the inventors used the NALM6 xenograft model treated with a suboptimal CAR T cell dose. The inventors generated CAR T cells with knockouts of PRDM1 or RHOG, or targeted the safe harbor locus PPP1R12C (intron1) as negative control (FIG. 16A-C). PRDM1, which was previously shown to improve CAR T cell therapy (Dai, Nat Biotechnol. 2023 September; 41(9):1239-125; Yoshikawa (2022), Blood 139 (14): 2156-72) and also identified as a hit in the genome-wide screens described in Example 1, was included as positive control. Prior to the in vivo experiment, CAR T cell cytotoxicity was confirmed in vitro by luciferase assays of a 24-hour co-culture with luciferase-expressing target cells (FIG. 16D). Regardless of the knockout, the killing ability of CAR T cells remained unchanged. This shows that RHOG knockout and its positive effect on proliferation does not diminish the T cell killing ability. Accordingly, this further indicates that RHOG knockout does not affect the killing machinery but rather concerns T cell stimulation and promotes T cell expansion and favorable T cell phenotypes (e.g., a memory phenotype and a metabolically active phenotype).

Both, RHOG and PRDM1 knockout CAR T cells injected at a suboptimal dose of 0.6 million cells per mouse decreased the tumor load compared to control CAR T cells edited at the Safe Harbor locus (FIG. 16E-G). The effect of the PRDM1 knockout was consistent with the previously reported findings. Surprisingly, for RHOG knockout CAR T cells, an up to 1000× fold reduction in tumor load until day 21 post treatment was observed (FIG. 16F-G), the effect being more pronounced and long-lasting than for PRDM1. Thus, in the long run, RHOG knockout CAR T cells controlled the tumor better than PRDM1 knockout CAR T cells. Importantly, only RHOG knockout CAR T cells significantly prolonged survival in the employed tumor model resulting in up to three times longer survival (FIG. 3H-1). Using a higher dose of 1.2 million CAR T cells, the inventors observed similar results (FIG. 16G-K). Moreover, and unexpectedly, cases of complete remission in mice treated with RHOG knockout CAR T cells were observed (FIG. 16G-K).

These results were obtained consistently in CAR T cells from 6 independent T cell donors for the 0.6 million/mouse CAR T cell dose and 3 independent T cell donors for the 1.2 million/mouse CAR T cell dose. The total editing efficiency at the RHOG locus averaged around ~60% (FIG. 19A), as measured by amplicon sequencing. It is envisioned that more efficient editing approaches such as RNPs will likely further improve tumor control. Thus, RHOG is a novel target for enhancing the therapeutic effect of CAR T cells.

### RHOG Knockout Enhances In Vivo Tumor Control by CAR T Cells in a Solid Tumor Model

To test the performance of RHOG knockout CAR T cells against solid tumors, the inventors established a xenograft model of subcutaneously injected CD19+K562 cells (FIG. 17A).

Throughout the CAR T treatment, the inventors monitored the tumor size with caliper measurements (FIG. 17B-D). While control CAR T cells with SafeHarbor editing did not affect tumor growth nor survival (FIG. 17B-C), surprisingly, a prolonged survival in the group treated with RHOG knockout CAR T cells was observed (FIG. 17D).

Thus, it has been surprisingly found in context of the present invention that RHOG is an effective target for enhancing T cell therapies in both liquid and solid tumors.

### RHOG Knockout CAR T Cells Display a Higher Proliferative and Metabolic Potential

The finding that RHOG knockout is an efficient booster of CAR T cells is particularly surprising, as several studies reported reduced T cell signaling in RHOG-deficient cells (Martinez-Martin, Immunity. 2011 Aug. 26; 35(2):208-22) or even immunodeficiency associated with RHOG dysfunction (Kalinichenko et al. (2021, Blood 137 (15): 2033-45). RHOG is a GTPase with a wide variety of functions reported in T cells (Martinez-Martin 2011)—from involvement in immune synapse formation to the regulation of T cell signaling and anergy. However, little is known about RHOG function in CAR T cells. Therefore, the inventors further investigated the molecular effects of RHOG inactivation in CAR T cells (FIG. 18A).

The inventors observed a strongly increased proliferative potential of RHOG knockout CAR T cells after TCR restimulation (FIG. 18B), in line with the genome-wide screen and in vivo screens described in Example 1. Moreover, RHOG and SafeHarbor knockout CAR T cells showed similar CD4:CD8 T cell composition and CAR expression level before injection (FIG. 19B-C).

To dissect the mechanism of RHOG knockout in CAR T cells, the inventors set up a time series experiment, repeatedly stimulating pan-CAR T cells with tumor cells for a total duration of 10 days. At timepoints representing acute CAR T stimulation (24, 72 hours stimulated once) and chronic overstimulation (168 and 240 hours, after 2 and 3 repeated stimulations), the inventors isolated CD4 or CD8 positive CAR T cells from the co-culture with antibody-bound magnetic beads and performed RNA-seq.

Unsupervised analysis revealed major differences between time points and cell types (FIG. 19D). Separation between CAR T cells with RHOG knockout or SafeHarbor edit was also notable at several timepoints. When comparing RHOG knockout CAR T cells to control, hundreds of differentially expressed genes were observed at each time point (FIG. 19E). Gene set enrichment analysis using all significant differentially expressed genes (adj. pval<0.05) revealed a strong enrichment of multiple cellular processes (FIG. 18C, 19F-H).

RHOG knockout CAR T cells under tumor cell stimulation upregulated multiple processes related to proliferation, biosynthesis and cell metabolism (FIG. 19C, 19G-H). DNA replication, cell cycle as well as the expression of S phase genes (FIG. 18D-E) were particularly enriched towards later time points (FIG. 18C-D, 19G-H), suggesting that RHOG knockout CAR T cells are more metabolically active and could sustain their proliferation potential longer. Moreover, these processes were typically upregulated in both CD4 and CD8 CAR T cells (FIG. 18C-D), suggesting a general effect of RHOG knockout on CAR T cells.

As CAR T cell differentiation status is a known predictor of clinical CAR T cell performance (see, e.g., Cappell, Nature Reviews Clinical Oncology volume 20, pages 359-371 (2023), the inventors profiled CD62L and CD45RO expression over 10 days of co-culture with tumor cells by flow cytometry. The inventors surprisingly observed an increased fraction of CD62L+CD45RO+ cells at multiple time points (FIGS. 18F, 19I-K), suggesting that RHOG knockout shifts the CAR T cell differentiation towards the central memory phenotype, which can support the increased T cell fitness and prolonged proliferation potential.

Taken together, the present surprising results suggest a multifaceted role of RHOG in CAR T cells, whereby RHOG knockout enhances immunotherapy by altering the overall metabolic and proliferative capacity of CAR T cells (FIG. 18G).

The inventors additionally profiled CAR T cell responses to stimulation with CD19+K562 cells in vitro by flow cytometry. No significant differences were observed in the levels of markers of CAR T cell signaling (CD19 for acquisition by trogocytosis, CD69 for T cell activation, CD107a for degranulation, IFNγ production), expression of the death receptor marker FAS (FIG. 6L), and exhaustion markers (PD1, LAG3, TIM3) (FIG. 19M), indicating that RHOG knockout does not alter these processes. Notably, the fact that RHOG knockout CAR T cells maintain their full killing ability and immediate T cell signaling makes RHOG a particularly attractive target for clinical use.

### Base Editing Screen

The inventors further performed a tiling screen with A->G and C->T base editing, with all possible mutations introduced into RHOG by different gRNAs and either Cas9 or one of 4 different CRISPR base editors (FIG. 20A-B). The inventors ran a proliferation screen (same setup as a genome-wide fitness screen), and found enriched gRNAs.

The dataset provides specific gRNA sequences that could be used to improve (CAR) T cells by modifying RHOG with base editors (FIG. 20C), as currently done in some clinical trials. Additionally, it provides evidence that specific amino acids can be targeted in RHOG (e.g. with inhibitors, etc). Mapping of identified amino acid targets on the RHOG 3D structure revealed the potential effect of mutations on the GTP binding activity, for example through interruption of H-bonds between Ser-168 or Cys-18 and GTP (FIG. 20D). Thus, targeting certain amino acids (e.g., Cys-18) and regions (e.g., GTP binding sites) of RHOG with mutations or inhibitors can be used to enhance T cell immunotherapies, which is a further surprising finding in context of the present invention.

TABLE 6
Examples of RHOG amino acids that could be targeted with mutations to enhance T cell immunotherapies
and corresponding gRNAs and CRISPR base editors that could be used to induce the mutations.
Top gRNAs with corresponding base editors are shown (same as highlighted in FIG. 20C).

| | | CRISPR | | Non-silent | | Position | |
|---|---|---|---|---|---|---|---|
| | | editor used | | mutations | Non- | of | z score for |
| | | for the top | | introduced | silent | target | corresponding |
| SEQ ID | | gRNA in | Editor | by CRISPR | mutation | amino | CRISPR |
| NO: | gRNA sequence | screen | type | editor | type | acids | editor |
| 5 | GCAGGCACGTCTT | ABEmax | A->G | C18R | Missense | 18 | 4.389 |
| | GCCCACA | | | | | | |
| 6 | GCCAAGCAGATCC | ABEmax | A->G | K147G; | Missense | 147, 148 | 2.939 |
| | ACGCTGT | | | Q148R | | | |
| 7 | GCAGGCACGTCTT | AncBE4max; | C->T | C18Y | Missense | 18 | 4.872; |
| | GCCCACA | SpRY-CBE | | | | | 3.317 |
| 8 | GAATGCTCAGCCCT | AncBE4max | C->T | S158L | Missense | 158 | 4.776 |
| | GCAACA | | | | | | |
| 9 | CGCACAGTGAACC | AncBE4max | C->T | T501 | Missense | 50 | 3.530 |
| | TGAACCT | | | | | | |
| 10 | CCACCACCACGCA | AncBE4max | C->T | V9M; V8I | Missense | 8, 9 | 3.377 |
| | CTTGATG | | | | | | |
| 11 | TCCCACAGGTTCAG | AncBE4max | C->T | W56Ter | Nonsense | 56 | 3.274 |
| | GTTCAC | | | | | | |
| 12 | GCCGAGGCTGTCC | AncBE4max | C->T | A172V | Missense | 172 | 3.245 |
| | GGGCTGT | | | | | | |
| 13 | GTGCGCTACCTCG | AncBE4max | C->T | R153C | Missense | 153 | 2.695 |
| | AATGCTC | | | | | | |
| 14 | CGCCCACGCTTGA | AncBE4max | C->T | G185K; | Missense | 184, 185 | 2.653 |
| | TCGGCGT | | | R184H | | | |
| 15 | GCCAAGCAGATCC | AncBE4max | C->T | Q148Ter | Nonsense | 148 | 3.174 |
| | ACGCTGT | | | | | | |
| 16 | GCTCTCAGGTCCTT | SpRY-CBE | C->T | R120K | Missense | 120 | 3.214 |
| | CTTGGT | | | | | | |
| 17 | GACACCCTACGGC | SpRY-CBE | C->T | T125I | Missense | 125 | 2.789 |
| | GCCTCAA | | | | | | |
| 18 | GATGTGCCCATCCT | SpRY-CBE | C->T | P109F | Missense | 109 | 2.716 |
| | GCTGGT | | | | | | |

TABLE 7
Base editors used for screening. ORF of reported base editors
were cloned into a vector for mRNA expression, and mRNA
was in vitro transcribed and electroporated into T cells.

| Application | ORF | CRISPR_PAM | Source |
|---|---|---|---|
| CRISPR base editing | ABEmax | NGG | Koblan et al., Nat |
| A->G | (NGG-ABE) | | Biotechnol. 2018 |
| | | | October; 36(9): 843-846 |
| CRISPR base editing | AncBE4max | NGG | Koblan et al., Nat |
| C->T | (NGG-CBE) | | Biotechnol. 2018 |
| | | | October; 36(9): 843-846 |
| CRISPR base editing | ABEmax7.10-SpRY | PAMless | Walton et al., Science. |
| A->G | (SpRY-ABE) | | 2020 Apr. 17; |
| | | | 368(6488): 290-296 |
| CRISPR base editing | CBE4max-SpRY | PAMless | Walton et al., Science. |
| C->T | (SpRY-CBE) | | 2020 Apr. 17; |
| | | | 368(6488): 290-296 |

### Methods Used to Produce the Experimental Data Described in the Present Example 2

CAR T cells were produced using two different experimental timelines (FIGS. 10, 16A and 17A) employing a lentivirus and mRNA delivery platform (for CAR and gRNA delivery stable integration and mRNA delivery of Cas9 or other CRISPR editors) as described in Example 1, and alternatively CRISPR RNP delivery. All experimental procedures and protocols are detailed below.

### Cloning CROP-Seq-CAR Screening Vectors

CAR T cells were produced using the CROP-seq-CAR lentiviral vector with the anti-CD19 4-1BB CAR.

In CROP-seq-CAR vectors, the gRNA is cloned into the 3′ LTR, making the system compatible with the CROP-seq technology for screens in single cells (Datlinger et al. 2017). CARs are expressed under the EF1α promoter, and composed of a leader peptide, single-chain variable fragment (scFv) antibody binding CD19 (clone FMC63 or SJ25C1), the hinge and transmembrane domains of CD8, intracellular co-stimulatory domains (CD28 or 41BB) and the CD3ζ chain (see FIG. 15 and SEQ ID NO: 1).

### Lentivirus Production

Lentivirus was produced using Lipofectamine 3000 (Invitrogen #L3000150) as described previously (Datlinger et al. 2017) followed by concentration using Lenti-X Concentrator (Takara #631232). All reagents and cell culture volumes were scaled accordingly for large-scale lentivirus production.

mRNA Production

DNA template for mRNA production was PCR-amplified from pIVTRup mRNA production plasmid input using Q5 Hot Start High-Fidelity 2× Master Mix (NEB #M0494L) and 0.5 μM primers: pIVTRup FWD AG 100 uM (5′-TTGGACCCTCGTACAGAAGCTAATACGACTCACTATAAGGAAATAAGAGAGAAAAGAAGAG-3′; SEQ ID NO: 36) and pIVTRup REV 100 uM (5′-TTTTTTTTTTTTTTTTTTTTTTTTTTTTTTTTTTTTTTTTTTTTTTTTTTTTTTTCTAGACCCTCACTTCC-3′; SEQ ID NO: 37), in a total volume of 50 μl. PCR product was cleaned up using QIAquick PCR purification Kit (Qiagen #28106). mRNA was produced using HiScribe T7 High Yield RNA Synthesis Kit (NEB #2040S) followed by DNase treatment according to manufacturer's instructions. Immediately after the incubation, mRNA from IVT reactions was cleaned up using the RNeasy Mini kit (Qiagen #74104). mRNA concentration was measured using Qubit RNA BR Assay Kit (Invitrogen #Q33223), and mRNA integrity was assessed by BioAnalyzer Agilent RNA 6000 Pico Kit (Agilent #5067-1513).

### T Cell Isolation

Human primary T cells for all experiments were isolated from fresh whole blood samples obtained from de-identified blood donors following written informed consent (Red Cross, Austria). The inventors used RosetteSep Human T Cell, CD4+ T Cell, or CD8+ T Cell Enrichment Cocktail (STEMCELL #15061, #15062, #15063) to isolate pan-CD3, CD4, or CD8 T cells, respectively. After incubation with antibody cocktail, cells were centrifuged using Lymphoprep (STEMCELL #7861) in SepMate-50 tubes (STEMCELL #85460), washed and treated with 1×RBC Lysis Buffer (eBioscience #00-4333-57). Isolated T cells were immediately stimulated and cultured.

### T Cell Culture

Complete cell culture medium included: CTS OpTmizer T-Cell Expansion SFM (ThermoFisher Scientific #A1048501/A3705001), where 1 L of CTS OpTmizer T-Cell Expansion Basal Medium was supplemented with 26 ml of CTS OpTmizer T-Cell Expansion Supplement, with 1% GlutaMAX Supplement (200 mM, 100×, Gibco #35050061), 1% Penicillin-Streptomycin (10,000 U/mL, Gibco #15140122), 2% Human AB Serum (heat-inactivated according to manufacturer's instructions, Sigma #H4522). The complete medium was supplemented with 12.1 ng/μl Human Recombinant IL-2 (STEMCELL #78145.2) right before use. Cells were incubated at 37° C. and 5% CO2.

For stimulation, T cells were seeded in medium supplemented with ImmunoCult Human CD3/CD28 T Cell Activator (STEMCELL #10990) and cultured for 2-3 days until complete medium exchange. For harvesting, T cells were spun down at 400 g for 5 min.

For optimization experiments, CD4 T cells were frozen after 1 week of pre-expansion in medium containing CTS OpTmizer T-Cell Expansion SFM with 20% Human AB Serum and 10% DMSO. Immediately after thawing, cells were stimulated and cultured. Cells were counted using CASY Cell Counter.

### T Cell Transduction

For transduction, T cells were harvested 2-3 days after stimulation and seeded in fresh medium. Lentivirus was added to the medium and mixed with cells. Medium was exchanged between 24 and 48 hours later. For single locus targeting, concentrated virus containing a single gRNA was added to the medium. For screens, the lentivirus titer was first quantified, and lentivirus was added to the medium for transduction.

### T Cell Electroporation

For electroporation, T cells were harvested 2-5 days after stimulation.

Electroporation using MaxCyte ATx system. Cells were pelleted, washed once with room temperature 1×PBS, washed once with room temperature MaxCyte Buffer, and then resuspended in MaxCyte Buffer. For mRNA electroporation, mRNA encoding CRISPR editors was added right before electroporation. mRNA encoding fluorescent markers or blasticidin resistance gene (BSD) was added as selectable marker.

For ribonucleoprotein (RNP) electroporation, synthetic complete gRNAs (Synthego/Invitrogen TrueGuide Synthetic gRNA) and Alt-R S.p. Cas9 Nuclease V3 (IDT #1081059) were mixed at a 2:1 molar ratio, and the mix was incubated for 30 min at room temperature for RNP complex formation. Right before electroporation, RNP was added and cells were pulsed.

Cells were mixed with mRNA or RNP and were transferred to MaxCyte cuvettes corresponding to the total electroporation volume, and the number of cuvettes used was scaled accordingly. Cuvettes were pulsed according to the manufacturer's instructions for T cells.

Electroporation using Amaxa Nucleofector II system. Cells were pelleted, washed once with room temperature 1×PBS. Right before electroporation, cells were resuspended in 100 μl of Electroporation Master Mix from the Amaxa Human T Cell Nucleofector Kit (Lonza #1002) and mixed with mRNA. Cells were transferred to the cuvettes and pulsed according to the manufacturer's instructions for T cells.

Immediately after electroporation, cells were transferred to fresh pre-warmed medium without puromycin.

### T Cell Ribonucleoprotein (RNP) Electroporation

Electroporation using MaxCyte ATx system. Cells were pelleted, washed once with room temperature 1×PBS, washed once with room temperature MaxCyte Buffer, and then resuspended in MaxCyte Buffer. Cells were mixed with mRNA and were transferred to MaxCyte cuvettes. Cuvettes were pulsed according to the manufacturer's instructions for T cells.

Electroporation using Amaxa Nucleofector II system. Cells were pelleted, washed once with room temperature 1×. Right before electroporation, cells were resuspended in 100 μl of Electroporation Master Mix from the Amaxa Human T Cell Nucleofector Kit (Lonza #1002) and mixed with mRNA. Cells were transferred to the cuvettes and pulsed according to the manufacturer's instructions for T cells.

Immediately after electroporation, cells were transferred to fresh pre-warmed medium without puromycin.

gRNAs for CRISPR Knockout

gRNAs were cloned and expressed from the hU6 promoter of CROP-seq-CAR screening vectors, or alternatively were ordered as the complete synthetic gRNAs containing chemical modifications for increased gRNA stability.

TABLE 8
List of exemplary gRNAs

| Purpose | Target | gRNA 5'-3' RNA sequence |
|---|---|---|
| 8-gRNA pool for RHOG | RHOG | GAAACAGAUGACGAAAACGU; CGCCGUCCUAUGA |
| knockout (SEQ ID NO: 19-26) | | GAACGUG; GCUGUAAUUGUCGAACACGG; CCUGA |
| | | ACCUGUGGGACACUG; GAGGGUAGGAGAGUGUA |
| | | CGG; GAAAACGUUGGUCUGAGGGU; UAGUUGUGU |
| | | AGCAGAUGAGC; AGCGCGCAGAGCGCAGUUGA |
| 1 gRNA selected for RHOG | RHOG | UAGUUGUGUAGCAGAUGAGC |
| knockout with RNPs | | |
| (SEQ ID NO: 25) | | |
| 8-gRNA pool for RHOG | SafeHarbor | AUAUGUCCCAGAUAGCACUG; AAGUGGUUGAUAA |
| knockout (SEQ ID NO: 27-34) | (PPP1R12C-intron1) | ACCCACG; UGUCACACUCCAGUUCACUG; AGAUAC |
| | | GCCUGGUUGCCCAG; GAAGCCAGUAGAGCUCAAA |
| | | G; GCGGAUCGAGACUGGCAACG; UGCUCAGUGGA |
| | | AACCACGAA; GAACCUGAGCUGCUCUGACG; |
| 1 gRNA selected for RHOG | SafeHarbor | AUAUGUCCCAGAUAGCACUG |
| knockout with RNPs (SEQ ID | (PPP1R12C-intron1) | |
| NO: 27) | | |
| Non-targeting gRNA for CAR- | Non-targeting | CACGUGUAGUCGAUCUGAU |
| CROP-seq vector used in | | |
| combination with RNPs | | |
| targeting RHOG or | | |
| SafeHarbor (SEQ ID NO: 35) | | |

### Establishing Cell Lines for the Co-Culture System

K562-CD19-mGFP and K562-CD20-mGFP cells were produced by lentivirus transduction of K562 cells with pLenti-C-CD19-mGFP-P2A-Puro and HEK_KBo pLenti-C-MS4A1-mGFP-P2A-Puro vectors at approximately 30% transduction efficiency, followed by selection with 2 μg/ml Puromycin (10 mg/ml, Gibco #A1113802), which was maintained throughout cell culture.

### Target Cell Culture

Suspension target cell lines K562 and NALM6 were cultured in RPMI 1640 (Gibco #11875085) with 10% FBS (Sigma #F7524) and 1% Penicillin-Streptomycin (10,000 U/mL, Gibco #15140122) Cells were split and culture medium was exchanged every 3-4 days. For genetically engineered cell lines, antibiotic selection was maintained accordingly throughout the cell culture.

### Cell Line Irradiation

K562-CD19-mGFP cells were resuspended in cell culture medium and irradiated with 100 Gy using a Yxlon X-Ray System. After irradiation, cells were spun down and washed once with fresh medium. Irradiated cells were either used fresh or were frozen at 20 M/ml in Freezing Medium (RPMI 1640 (Gibco #11875085), 5% FBS (Sigma #F7524) #, 5% DMSO (Sigma #D2650)).

### Readout for Fitness Screens

4-8 days after electroporation cells were restimulated. For TCR stimulation, cells were seeded at 1 M/ml in medium supplemented with ImmunoCult Human CD3/CD28 T Cell Activator (STEMCELL #10990) and cultured for 3 days before medium exchange and ImmunoCult removal. For CAR stimulation, cells were seeded at 1 M/ml in the medium containing 1 M/ml K562-CD19-mGFP target cells irradiated with 100 Gy prior to stimulation. The medium contained 0.5 μg/ml puromycin during the whole time. Medium was exchanged every 2-4 days. On day 7-21 post electroporation, cells were harvested for the analysis of gRNA representation.

gRNA Amplification and Sequencing from Genomic and Plasmid DNA

Genomic DNA was isolated using DNeasy Blood & Tissue Kit (Qiagen #69506) or QIAamp DNA Blood Maxi Kit (Qiagen #51194) depending on the input cell number. For lysis, we used AL buffer for freshly collected cells and ATL buffer for sorted fixed cells. In both cases, lysates were incubated with proteinase K for 10 min at 56° C., and fixed cells were additionally incubated for 4-12 hours at 65° C. for DNA decrosslinking.

Amplification and indexing of purified genomic DNA were performed in 50 μl qPCR reactions as described for CROP-seq (Datlinger et al 2017) using primers (CROPseq_libQC_i5 and CROPseq_libQC_i7) to amplify a sequencing-ready product. Amplification was stopped once it reached the exponential phase to avoid library over-amplification. Multiple PCR reactions were set up to retain a high library representation. Each PCR reaction contained up to 2 μg of genomic DNA, and the number of PCR reactions was scaled up to process the entire input material. PCR reactions amplified from the same sample were pooled together, and a minimum of 10% of the total volume were used for purification.

For plasmid libraries, we set up multiple PCRs with input depending on the library size and followed the same procedure for amplification and purification.

PCR-amplified gRNAs were cleaned up using bead-based purification with Mag-Bind TotalPure NGS Beads (Omega Bio-tek #M1378-01) according to manufacturer's instructions by a stepwise 0.45× large fragment removal to remove gDNA, and 1× small fragment removal to remove primer dimers.

gRNA Sequencing

The desired read number was calculated based on the cell number collected and estimated fraction of cells containing gRNA in the sample to obtain at least 4-10 reads per cell. Concentration was measured in a Qubit HS assay (Invitrogen #Q32854), and 0.25 ng were analyzed on an Agilent High Sensitivity DNA chip (Agilent #5067-4626) to confirm the purity of the expected single band at 282 bp. Libraries were diluted to 3.5 nM with EB Buffer+0.1% Tween and pooled according to the desired read number and sample purity. Libraries were sequenced with 1% PhiX on the Illumina Novaseq 6000 platform using a 100-cycle v1.5 flow cell with a read configuration of 122 Read1+8 Index1+8 Index2 for pooled CRISPR screens.

### Luciferase Assay on Microwell Plates

CAR T cells were co-incubated with luciferase-expressing K562-CD19-mGFP for 18 hours. For the readout, cells were lysed in 384-well assay plate (Corning #3707) using the Steady-Glo Luciferase Assay System (Promega #E2510), and assessed with the Perkin Elmer Victor x3 2030 Multilabel Reader.

### NALM-6 Xenograft Systemic Leukemia Tumor Model

NOD/SCID/IL-2Rγ-null (NSG) mice at 8-12 weeks of age were intravenously injected with 0.5 million NALM-6 cells (clone G5, ATCC #CRL-3273). CAR T cells were administered by intravenous injections 5 days later. For single knockout validation, male mice were injected with 0.6 million CAR T cells. For in vivo screens, female mice were injected with 1 million CAR T cells. Mice were followed over time by bioluminescence imaging, weight measurements and were monitored for any signs of morbidity.

### IVIS Bioluminescence Imaging

XenoLight D-Luciferin K+salt (PerkinElmer #122799) was diluted to 30 mg/ml in sterile 1×PBS and administered to mice at 150 mg/kg body weight by intraperitoneal injection. Mice were anesthetized with isoflurane and the bioluminescence measurement was performed 15 minutes post injection using the IVIS Spectrum In Vivo Imaging System (PerkinElmer #124262). Bioluminescence was quantified using Living Image Analysis Software (PerkinElmer).

### RNA-Seq

RNA-seq was performed on CAR T cells produced from CD3+ T cells. CD4+ or CD8+ CAR T cells were selected from either the total T cell pool or the mixture with irradiated tumor cells by using EasySep Human CD4 Positive Selection Kit II (STEMCELL #17852) and EasySep Human CD8 Positive Selection Kit II (STEMCELL #17853). RNA was isolated using the Monarch Total RNA Miniprep Kit (NEB #T2010S) from at least 200,000 cells lysed and stored at −80° C. in 300 μl of the Protection Reagent. Sequencing libraries were prepared with the NEBNext Ultra II Directional RNA Library Prep Kit for Illumina (NEB #E7760L) according to manufacturer's instructions and sequenced with paired-end 50-bp reads.

### Base Editing Screening

For PAMless tiling screens, all possible gRNAs were used for the gene.

log FC scores were obtained by comparing samples from Day 14 (readout) and Day 0 (electroporation) using MAGeCK RRA with parameters --norm-method median, --remove-zero both, --remove-zero-threshold 0, --paired, and --variance-estimation-samples, pairing samples by T cell donor (n=2) and using samples at Day 0 for variance estimation.

Structures were downloaded from the protein database (PDB) and the 15 amino acids corresponding to the most impactful gRNAs were visualized as stick representation using PyMOL. Hydrogen bonds were computed between the highlighted amino acids and the small molecule reagents using the default PyMOL command distance hbonds, small_molecules, top15_aas, mode=2.

### Example 3. Methods for Producing CAR T Cells with RHOG Inactivation Optimized for Clinical Use

Multiple clinical trials are currently ongoing, where one or more targets in (CAR) T cells are inactivated to further improve their performance, in particular in the field of cancer immunotherapy. Production methods employed in these studies are well known in the art and can also be used to produce CAR T cells with RHOG inactivation.

Suitable methods can be derived from, inter alia, the following research programs
CRISPR-engineered T cells in patients with refractory cancer, knockouts of TRAC, TRBC, PDCD1 (Stadtmauer, Science. 2020 Feb. 28; 367(6481):eaba7365). This is an example of CRISPR mediated triple knockout in therapeutic T cells and the paper and supplement describe clinical grade production methods to obtain such cells.
CRISPR knockout of PD1 (Hu, EClinicalMedicine. 2023 May 18:60:102010, a key inhibitory receptor of T cells and exhaustion marker
CRISPR knockout of TRAC and CD52 (Ottaviano, Sci Transl Med. 2022 Oct. 26; 14(668):eabq3010). Refer, in particular, to CD52 KO by TALEN.
Base-Edited CAR7 T Cells for Relapsed T-Cell Acute Lymphoblastic Leukemia (Chiesa, N Engl J Med. 2023 Sep. 7; 389(10):899-910). This is an example using base editing of three genes, and contains detailed methods that a person skilled in the art could use to produce base edited T cells for clinical applications.

The above-cited documents are incorporated herein by reference in their entirety.

RHOG inactivated CAR T cells for clinical use may be, for example, prepared as follows (in line with the protocol described in Stadtmauer (2020)):

Obtain autologous T cells by apheresis and elutriation. Seed the cells in culture medium supplemented with cytokines (IL-2, IL-7, IL-15 or others, single or in combination) and expand. On day −2, electroporate RNP complexes containing CRISPR modifier(s) and gRNA(s) targeting RHOG (single gRNA or a mixture of effective gRNAs). Following electroporation, culture at lower temperature (cold shock) can help T cells recover. On day 0, shift temperature back to 37 C and activate T cells, for example by anti-CD3, anti-CD28 stimulation with antibodies.

On day 1-2, deliver CAR construct to T cells, e.g. by lentiviral transduction, delivery of transposomes, delivery of stably transmitted plasmids. Expand T cells in static culture or bioreactor systems. Harvest T cells, formulate and optionally cryopreserve.

As another example, RHOG inactivated CAR T cells for clinical use may be prepared as follows (in line with the protocol described in Chiesa (2023):

Autologous T cells are obtained by apheresis and elutriation, and cells are loaded into a CliniMACS Prodigy device and then activated using, e.g. TransAct reagent in TexMACS media with 3% human serum and IL-2. After 48 hours, cells are removed from the device and electroporated with mRNA encoding base editors along with corresponding gRNA(s) introducing RHOG inactivating mutations (e.g. using a Lonza 4D nucleofector). After electroporation, cells are returned to the Prodigy chamber and the following day are transduced with a lentivirus preparation encoding a synthetic immune receptor (e.g., anti-CD19 CAR). Cells are further expanded with fresh TexMACS 3% HS and IL-2 added (e.g. for a duration of 11 days+/−2 days). The Prodigy keeps cells at 5% C02 and 37° C. with shaking to facilitate optimal gas exchange. The next day the T cell product is formulated and cryopreserved.

Furthermore, all nucleotide and amino acid sequences used in the invention can be optimized to reduce immunogenicity in the clinic.

### LIST OF REFERENCES

1. Datlinger, P. et al. Pooled CRISPR screening with single-cell transcriptome read-out. Nat. Methods 14, 297-301 (2017).
2. Shifrut, E. et al. Genome-wide CRISPR Screens in Primary Human T Cells Reveal Key Regulators of Immune Function. Cell 175, 1958-1971.e15 (2018).
3. Schmidt, R. et al. CRISPR activation and interference screens decode stimulation responses in primary human T cells. Science 375, eabj4008.
4. Wang, D. et al. CRISPR Screening of CAR T Cells and Cancer Stem Cells Reveals Critical Dependencies for Cell-Based Therapies. Cancer Discov. 11, 1192-1211 (2021).
5. Hamieh, M. et al. CAR T cell trogocytosis and cooperative killing regulate tumour antigen escape. Nature 568, 112-116 (2019).
6. Roelands, J. et al. Long-Chain Acyl-CoA Synthetase 1 Role in Sepsis and Immunity: Perspectives From a Parallel Review of Public Transcriptome Datasets and of the Literature. Front. Immunol. 10, 2410 (2019).
7. Catala-Rabasa, A. et al. High ACSL5 Transcript Levels Associate with Systemic Lupus Erythematosus and Apoptosis in Jurkat T Lymphocytes and Peripheral Blood Cells. PLoS ONE 6, e28591 (2011).
8. Guedan, S., Ruella, M. & June, C. H. Emerging Cellular Therapies for Cancer. Annu. Rev. Immunol. 37, 145-171 (2019).
9. Mao, A. S. & Mooney, D. J. Regenerative medicine: Current therapies and future directions. Proc. Natl. Acad. Sci. 112, 14452-14459 (2015).
10. Shah, N. N. & Fry, T. J. Mechanisms of resistance to CAR T cell therapy. Nat. Rev. Clin. Oncol. (2019) doi:10.1038/s41571-019-0184-6.
11. Larson, R. C. & Maus, M. V. Recent advances and discoveries in the mechanisms and functions of CAR T cells. Nat. Rev. Cancer 21, 145-161 (2021).
12. McGowan, E. et al. PD-1 disrupted CAR-T cells in the treatment of solid tumors: Promises and challenges. Biomed. Pharmacother. 121, 109625 (2020).
13. Stadtmauer, E. A. et al. CRISPR-engineered T cells in patients with refractory cancer. Science 367, eaba7365 (2020).
14. Fraietta, J. A. et al. Disruption of TET2 promotes the therapeutic efficacy of CD19-targeted T cells. Nature 558, 307-312 (2018).
15. June, C. H. & Sadelain, M. Chimeric Antigen Receptor Therapy. N. Engl. J. Med. 379, 64-73 (2018).
16. Tsimberidou, A.-M. et al. T-cell receptor-based therapy: an innovative therapeutic approach for solid tumors. J. Hematol. Oncol. J Hematol Oncol 14, 102 (2021).
17. Brogdon, J., June, C. H., Loew, A., Maus, M. & Scholler, J. Treatment of cancer using humanized anti-cd19 chimeric antigen receptor.
18. Doench, J. G. Am I ready for CRISPR? A user's guide to genetic screens. Nat. Rev. Genet. 19, 67-80 (2018).
19. Adamson, B. et al. A Multiplexed Single-Cell CRISPR Screening Platform Enables Systematic Dissection of the Unfolded Protein Response. Cell 167, 1867-1882.e21 (2016).
20. Dixit, A. et al. Perturb-Seq: Dissecting Molecular Circuits with Scalable Single-Cell RNA Profiling of Pooled Genetic Screens. Cell 167, 1853-1866.e17 (2016).
21. Jaitin, D. A. et al. Dissecting Immune Circuits by Linking CRISPR-Pooled Screens with Single-Cell RNA-Seq. Cell 167, 1883-1896.e15 (2016).
22. Pierce, S. E., Granja, J. M. & Greenleaf, W. J. High-throughput single-cell chromatin accessibility CRISPR screens enable unbiased identification of regulatory networks in cancer. Nat. Commun. 12, 2969 (2021).
23. Liscovitch-Brauer, N. et al. Profiling the genetic determinants of chromatin accessibility with scalable single-cell CRISPR screens. Nat. Biotechnol. 39, 1270-1277 (2021).
24. Pagliano, O. et al. Tim-3 mediates T cell trogocytosis to limit antitumor immunity. J. Clin. Invest. (2022) doi:10.1172/JC1152864.
25. Prommersberger, S. et al. CARAMBA: afirst-in-human clinical trial with SLAMF7 CAR-T cells prepared by virus-free Sleeping Beauty gene transfer to treat multiple myeloma. Gene Ther 28, 560-571 (2021). https://doi.org/10.1038/s41434-021-00254-w
26. Kebriaei, P. et al. Gene Therapy with the Sleeping Beauty Transposon System, Trends in Genetics, Volume 33, Issue 11, 2017, 852-870. https://doi.org/10.1016/j.tig.2017.08.008.
27. Freitas, K. A., et al. “Enhanced T cell effector activity by targeting the Mediator kinase module.” Science 378.6620 (2022): eabn5647.

1. A method of preventing, ameliorating, or treating a disease or disorder, said method comprising administering an effective amount of a T cell or a population thereof to a subject in need thereof, wherein said T cell comprises
(a) a RHOG modulator that is suitable for suppressing, impairing or abolishing the expression and/or activity of RHOG in a T cell, and/or
(b) an inactivating mutation in at least one allele of an endogenous RHOG gene.

2. (canceled)

3. The method of claim 1, wherein said RHOG modulator comprises
(i) a gene editing molecule and, optionally, a gene editing modifier, and/or
(ii) an RNA interference (RNAi) molecule.

4. The method of claim 3, wherein said gene editing molecule comprises a CRISPR guide RNA (gRNA) and said gene editing modifier comprises a CRISPR modifier, or said gene editing modifier comprises a transcription activator-like effector nuclease (TALEN) and/or a zinc finger nuclease.

5. The method of claim 4, wherein said CRISPR modifier comprises
an endonuclease,
a prime editor,
a base editor, and/or
(IV) an endonuclease deficient protein fused to a repressive domain.

6. (canceled)

7. The method of claim 1, wherein said RHOG modulator is suitable for introducing an inactivating mutation in an endogenous RHOG gene in said T cell.

8-11. (canceled)

12. The method of claim 1, wherein said inactivating mutation comprises at least one deletion, insertion and/or point mutation in at least one allele of an endogenous RHOG gene.

13-14. (canceled)

15. The method of claim 1, wherein said inactivating mutation suppresses, impairs or abolishes the expression of the RHOG gene or RHOG protein in said T cell as compared to a T cell comprising exactly two alleles of a RHOG gene each having a sequence as set forth in SEQ ID NO: 2; and/or wherein said inactivating mutation suppresses, impairs or abolishes the activity of the RHOG protein in said T cell as compared to a RHOG protein consisting of a sequence as set forth in SEQ ID NO: 4.

16-17. (canceled)

18. The method of claim 1, wherein said T cell comprises a synthetic antigen receptor.

19. The method of claim 18, wherein said synthetic antigen receptor is a chimeric antigen receptor (CAR).

20. The method of claim 18, wherein said synthetic antigen receptor comprises
an antigen-binding domain, a hinge domain,
a transmembrane domain, and
an intracellular signalling domain.

21. The method of claim 20, wherein
(i) said antigen-binding domain is a single-chain variable fragment (scFv), and/or specifically binds to CD19, BCMA, GD2, HER2, CD123, CD138, CD20, CD22, CD38 CDK5, IgK, LeY, NKG2D, ROR1, WT1, c-met, CAIX, CD70, CEA, EGFR, EpCAM, EphA2, FAP, GD2 HER2, IL13RA2, LeY, MAGEA3, MAGEA4, MART1, Mesothelin, MUC1, MUC16, NY-ESO-1, PD-L1, PSCA, PSMA, VEGFR2, CD171, EGFRVvIII, FR, Glypican-3, CD23, CD32B, CD70, CD72, CD37, CD79b, CD133, Fc R, Siglec-6, TSLPR, BAFF-R, GPRC5D, LMP1, SLAMF7, or GPRC5D;
(ii) said transmembrane domain and said hinge domain are from CD8; and/or
(iii) said intracellular signalling domain comprises an activating domain and at least one co-stimulatory domain, wherein said activating domain comprises a CD3ζ chain, at least one ITAM from CD3ζ, an FceRIg chain, or at least one ITAM from FceRIg, and/or said at least one co-stimulatory domain comprises at least one co-stimulatory domain selected from the co-stimulatory domains of CD28, CD27, OX40, 4-1BB, ICOS, CD40, CD40L, Toll-like receptors (TLRs), MyD88, and IL-15Rα.

22-27. (canceled)

28. The method of claim 19, wherein said CAR is a polypeptide comprising an scFv, a hinge domain from CD8, a transmembrane domain from CD8, and an intracellular signalling domain comprising co-stimulatory domains of CD28 and 4-1BB and a CD3ζ.

29-30. (canceled)

31. The method of claim 1, wherein said T cell (i) is a CD8+ T cell or a CD4+ T cell; (ii) is a primary T cell, from a T cell line, or derived from a pluripotent stem cell; (iii) is an autologous T cell or an allogeneic T cell; (iv) has a memory phenotype; and/or (v) is activated.

32-43. (canceled)

44. The method of claim 1, wherein said disease or disorder is a cancer, a tumour or a cancerous disease, an autoimmune disorder, a chronic bacterial or viral infection, or Graft Versus Host Disease (GVHD).

45. The method of claim 44, wherein
(a) said cancer, tumour or cancerous disease is
(I) a blood cancer, a liquid tumor or a hematological tumor selected from the group consisting of: an acute Lymphocytic Leukemia (ALL), a B-Cell Leukemia such as B-Cell Acute Lymphocytic Leukemia, B-Cell Chronic Lymphocytic Leukemia, Nodal Marginal Zone B-Cell Lymphoma, Extranodal Marginal Zone B-Cell Lymphoma (Mucosa-Associated Lymphoid Tissue or MALT-Lymphoma), or Splenic Marginal Zone B-Cell Lymphoma, a T-Cell Leukemia such as T-Cell Acute Lymphocytic Leukemia, Chronic Lymphocytic Leukemia (CLL) (e.g. Refractory CLL or Relapsed CLL), an Acute Myelocytic Leukemia (AML) (e.g. Refractory AML or Relapsed AML), a Chronic Myelocytic Leukemia (CML), or Hairy Cell Leukemia, a B-Cell Lymphoma such as Diffuse Large B-Cell Lymphoma, Marginal Zone B-cell Lymphoma, Hodgkin Lymphoma (in particular, B-Cell Hodgkin Lymphoma), Non-Hodgkin Lymphoma (in particular, B-Cell Non-Hodgkin Lymphoma), Primary Mediastinal B-Cell Lymphoma, a T-cell Lymphoma such as a Peripheral T-Cell Lymphoma (PTCL), an Angioimmunoblastic T-Cell Lymphoma (AITL), a Follicular Lymphoma, a Mantle Cell Lymphoma, Burkitt Lymphoma, Anaplastic Large Cell Lymphoma (ALCL), a Lymphoblastic Lymphoma such as Acute Lymphoblastic Lymphoma, Natural Killer Cell Lymphomas, Waldenstrom Macroglobulinemia, Multiple Myeloma (e.g. Refractory Multiple Myeloma or Relapsed Multiple Myeloma), a Myelodysplastic Syndrome, a Plasma Cell Neoplasm, Sezary Syndrome, Mycosis Fungoides, and POEMS Syndrome (Crow-Fukase Syndrome);
(II) a solid tumor selected from the group consisting of: Pancreatic Cancer, Metastatic Pancreatic Cancer, Gastric Cancer, Ovarian Cancer such as Epithelial Ovarian Cancer, Lung Cancer such as Small-Cell Lung Cancer or Non-Small Cell Lung Cancer, Colorectal Cancer such as Metastatic Colorectal Cancer, Glioblastoma Multiforme (GBM) such as Recurrent Glioblastoma Multiforme (GBM), Hepatocellular Carcinoma, Breast Cancer such as Metastatic Breast Cancer, Human Epidermal Growth Factor Receptor 2 Positive Breast Cancer (HER2+ Breast Cancer) or Triple-Negative Breast Cancer (TNBC), Neuroblastoma, Prostate Cancer such as Castration-Resistant Prostate Cancer (CRPC) or Metastatic Castration-Resistant Prostate Cancer (mCRPC), Esophageal Cancer, Liver Cancer, Melanoma such as Metastatic Melanoma, Bile Duct Cancer (Cholangiocarcinoma), Sarcoma such as Osteosarcoma, Glioma, Malignant Mesothelioma such as Malignant Pleural Mesothelioma, Endometrial Cancer, Kidney Cancer such as Renal Cell Carcinoma (e.g. Metastatic Renal Cell Carcinoma), Fallopian Tube Cancer, Adenocarcinoma Of The Gastroesophageal Junction, Pancreatic Ductal Adenocarcinoma, Cervical Cancer, Bladder Cancer, Nasopharyngeal Cancer, Peritoneal Cancer, and Head And Neck Cancer;
(b) said autoimmune disorder is Systemic Lupus Erythematosus, Systemic Sclerosis or Lupus Nephritis; and/or
(c) said chronic bacterial or viral infection is Human Immunodeficiency Virus (HIV) Infection.

46. An engineered mammalian cell or a population thereof, said engineered mammalian cell comprising the following (I) and (II):
(I) (a) a RHOG modulator that is suitable for suppressing, impairing or abolishing the expression and/or activity of RHOG in a mammalian cell, in particular, a T cell, and/or (b) an inactivating mutation in at least one allele of an endogenous RHOG gene; and
(II) a synthetic antigen receptor.

47-48. (canceled)

49. The engineered mammalian cell or population thereof according to claim 46, wherein said engineered mammalian cell is a T cell, a T cell precursor cell, or a pluripotent stem cell.

50-102. (canceled)

103. The method of claim 44, wherein said disease or disorder is a leukemia, a lymphoma, or a myeloma.

104. A pharmaceutical composition comprising a T cell or a population thereof, wherein said T cell comprises
(a) a RHOG modulator that is suitable for suppressing, impairing or abolishing the expression and/or activity of RHOG in a T cell, and/or
(b) an inactivating mutation in at least one allele of an endogenous RHOG gene.

105. A CAR T cell population in which expression of RHOG, HAVCR2, VAMP4, SLC35B3, TRAF1, NOTCH2, PHF8, DUSP6, DAW1, ZNF16, TCF4, PRSS3, LACTB, TMTC3, CD300LB, MORC3 and/or CDKN2A is suppressed.
